(19) United States
(12) Patent Application Publication
(10) Pub. No.: US 2025/0281584 A1
(43) Pub. Date: Sep. 11, 2025

(54) ACTIVE VACCINATION FOR THE TREATMENT OF NGF-RELATED DISORDERS

(71) Applicant: Boehringer Ingelheim Vetmedica GmbH, Ingelheim am Rhein (DE)

(72) Inventors: Laurent Bernard FISCHER, Ingelheim am Rhein (DE); Andreas GALLEI, Ingelheim am Rhein (DE)

(21) Appl. No.: 19/068,745

(22) Filed: Mar. 3, 2025

(30) Foreign Application Priority Data
Mar. 4, 2024 (EP) 24161057

(51) Int. Cl. A61K 39/00; A61K 9/00; A61K 39/385; A61P 19/02; C12N 7/00; C12N 15/70

(52) U.S. Cl. CPC A61K 39/00113; A61K 9/0019; A61K 39/385; A61P 19/02; C12N 7/00; C12N 15/70; A61K 2039/54; A61K 2039/545; A61K 2039/552; A61K 2039/575; A61K 2039/6075; C12N 2770/14023; C12N 2770/14034

(57) ABSTRACT

The present invention relates to veterinary compositions comprising NGF antigen linked to modified virus-like particles (VLPs) of Cucumber Mosaic Virus (CMV), in particular to modified VLPs of CMV comprising chimeric CMV polypeptides for use in a method of treating a NGF-related disorder in canine, in particular in the treatment of pain, such as for example pain associated with osteoarthritis (OA) in dogs

### SEQUENCE LISTING

The instant application includes a Sequence Listing which has been submitted electronically in XML format and is hereby incorporated by reference in its entirety. The Sequence Listing, created on Mar. 3, 2025, is named “0192-0233US1 Sequence Listing” and is 59,625 bytes in size.

The present invention relates to compositions comprising NGF antigen linked to virus-like particles (VLPs) of Cucumber Mosaic Virus (CMV), in particular to modified VLPs of CMV comprising chimeric CMV polypeptides for use in a method of treating a NGF-related disorder in canine, in particular in a method of treating pain, such as for example nociceptive, inflammatory-related, postsurgical pain, pain associated with musculoskeletal diseases, pain associated with degenerative joint disease and/or osteoarthritis (OA)-associated pain. The pain can be acute or chronic pain. Moreover, the present invention provides highly immunogenic vaccine compositions comprising NGF antigen linked to virus-like particles (VLPs) of Cucumber Mosaic Virus (CMV), in particular to modified VLPs of CMV comprising chimeric CMV polypeptides for use in the active immunization of canine against NGF-related disorder, in particular against pain.

### RELATED ART

Nerve Growth Factor (NGF) was originally discovered as a critical factor for the development and maintenance of sensory and sympathetic neurons in the developing nervous system. Indeed, during prenatal and early postnatal periods, NGF is required for survival of both sensory and sympathetic neurons. However, in adults, the main role of NGF in the periphery shifts from trophic support of sensory and sympathetic neurons to modulation of nociceptive neuronal activity. Preclinical and clinical research over the past several decades has clearly demonstrated the important role of NGF in nociceptor sensitization in a wide variety of both acute and chronic pain states including OA pain.

Pain associated to osteoarthritis (OA) causes decreased and altered mobility, which both subsequently lead to local and distant deterioration of the musculoskeletal system. What's more, the pathological processes of OA, such as joint capsule thickening and fibrosis, accelerate musculoskeletal changes that impact motion abilities. Finally, the ongoing nociceptive input into the CNS results in somatosensory system changes and central sensitization, which also contribute to the perception of pain. In humans, the combined effects of pain, central sensitization and activity impairment may have negative effects on the affective state, heightening anxiety, depression, sleep impairment and cognitive dysfunction. Very similar consequences are also occurring in dogs suffering from OA.

Osteoarthritis (OA) is a slowly progressive degenerative joint disease characterized by whole-joint structural changes including articular cartilage, synovium, subchondral bone and periarticular components, which frequently lead to pain and loss of joint function. In canine (dogs), it is considered to primarily affect the hip, stifle and elbow joints, although no comprehensive, prospective studies of the prevalence of canine OA throughout the skeleton have been performed. OA is commonly initiated early in life by developmental disease (e.g., hip dysplasia), but many other factors play a role in its development, including diet, genetics, environment (including traumatisms), obesity and age. OA is associated with clinical signs in a large percentage of the canine population, with an estimated minimum of 20% to 30% of dogs affected clinically. The disease is currently incurable with negative consequences related to pain, mobility impairment and decreased quality of life (QoL).

Currently, pharmacological treatment of pain centers around non-steroidal anti-inflammatory drugs (NSAIDs). These are used to relieve pain and to promote functional improvement. Globally, several NSAIDs are approved in dogs. Despite their widespread use and obvious benefit in many cases, NSAIDs are not always sufficiently effective as monotherapy. Moreover, ongoing treatments often have adverse effects, including serious gastro-intestinal and kidney toxicity.

Recently, anti-NGF therapeutic mAbs has been proposed to alleviate pain in canine OA patients (WO2022/076712). However, despite the benefit of mAb treatments, there are still certain challenges associated with mAb treatments (e.g., high costs, risk of anti-drug antibodies (ADA), risk of anaphylactic reactions and local reactions due to the substantial amounts of protein per dose). Furthermore, mAbs show limited duration of efficacy due to its degeneration and limited bioavailability.

Virus-like particles (VLPs) have been proposed and used as vaccine technology, in particular as immunological carriers for inducing immune responses against conjugated antigens (Zeltins A, Mol Biotechnol (2013) 53:92-107; Jennings G T and Bachmann M F, Annu Rev Pharmacol Toxicol (2009) 49:303-26, Jennings G T and Bachmann M F, Biol Chem (2008) 389:521-536). Recently, Cucumber Mosaic Virus (CMV, family Bromoviridae, genus Cucumovirus) virus-like particles (CMV VLPs) has been described using chemical linker coupling technology (WO2016/062720) or fusion technology (WO2020/128037) to present antigens including self-antigens on their surface and to elicit neutralizing antibody responses against certain targets.

However, an effective treatment of pain in dogs, in particular by active immunization against endogenous NGF using CMV VLPs has not been shown. Such effective treatment has to ensure that (i) sufficient levels of antibodies are reached, and (ii) in particular in case of treatments against endogenous NGF protein, the induced antibody responses is controllable and irreversible. There is no information that NGF-related disorder, in particular pain, for example pain associated with degenerative joint disease such as OA associated pain can be controlled by active treatment with NGF-containing VLPs, in particular with NGF-containing VLPs of CMV.

Despite this, there are challenges and requirements that have to be taken into account for the development of a drug, in particular for clinical trial testing, product registration, market launch and commercial supply needs. Hereby, controlling product characteristics such as stability, shelf-life, solubility, manufacturability including scalability, safety, potency, bioavailability and other pharmacological properties are particularly to be mentioned and are key elements of the chemistry, manufacturing and control (CMC) process necessary for the cost-effective provision of these products in sufficient amounts for such eventual needs (Pham N G, Int J Pharm, 2020, 585:119523). The stability of VLP based vaccines even under various conditions required for an efficient CMC process is of relevance. A further undesired occurrence and problem negatively impacting product characteristics is the aggregation of biopharmaceuticals and vaccines, respectively (Roberts C J, Current Opinion in Biotechnology, 2014, 30:211-217). While an aggregated vaccine may still be capable of eliciting an immune response, provided its native structure is maintained, and even though it may thus still be suitable for some laboratory studies, it is less acceptable for GMP products produced for clinical studies and the market.

Therefore, based on the current limitations in the control of NGF-related disorder such as for example pain, in particular in controlling OA associated pain, whether acute or chronic pain, there is an unmet need in providing effective therapies to control NGF-related disorders that arise and meet the requirements for eventual product registration and market launch.

### SUMMARY OF THE INVENTION

The present invention provides for compositions for the treatment of NGF-related disorders, in particular for the treatment of pain in canine. In particular, the present invention provides for compositions for the active immunization of canine, against endogenous NGF protein of such immunized canine to raise high-level and long-lasting neutralizing antibodies against such endogenous NGF protein in such canine for the active pain management. In particular, said pain is selected from the group of nociceptive pain, inflammatory pain, postsurgical pain, pain associated with musculoskeletal diseases, pain associated with degenerative joint disease, and/or osteoarthritis (OA)-associated pain, preferably acute, chronic and/or refractory related pain.

It has been surprisingly found, that the use of the composition described herein, in particular in form of an active immunization of canine against endogenous NGF protein is advantageous compared with the prior art treatments. For instance, the treatment provided herein induces long-lasting neutralizing anti-NGF antibody titers by transiently overcoming the natural immune tolerance. Moreover, these long-lasting neutralizing anti-NGF antibodies eliminate the need for frequent administrations of more expensive recombinant monoclonals (mAb) as well pharmaceuticals.

Moreover, the animal models used herewith have not only shown that the administration of the NGF containing Cucumber Virus (CMV) virus-like particle (VLP) compositions provided herewith raised high titers of long-lasting neutralizing anti-NGF antibodies in vivo, they also provided evidence that the active immunization with the NGF containing CMV VLP compositions surprisingly lessened NGF-related disorders, in particular acute as well as chronic pain. It was even more surprisingly, that treatment with the NGF containing CMV VLP compositions was superior in pain management as compared to the antibody treatment and the treatment with NSAIDs. Furthermore, even if the treatment addresses a self-protein (NGF protein), the induced immune response against self-NGF protein did not cause any side effects in canine, while being effective in managing NGF-related disorder, in particular pain.

Thus, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site.

In particular, the NGF-related disorder is pain, preferably pain is selected from the group consisting of nociceptive pain, inflammatory pain, postsurgical pain, pain associated with musculoskeletal diseases, pain associated with degenerative joint disease and/or osteoarthritis (OA)-associated pain. Data have shown that such treatment can reduce acute, chronic and refractory pain.

Thus according to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site; and wherein the NGF-related disorder is pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site; and wherein the NGF-related disorder is pain selected from the group consisting of nociceptive pain, inflammatory pain, postsurgical pain, pain associated with musculoskeletal diseases, pain associated with degenerative joint disease, and/or osteoarthritis (OA)-associated pain, preferably wherein such pain is OA-associated pain.

The animal data provided herein has shown that the NGF containing CMV VLP compositions are able to treat, in particular alleviate acute, chronic as well as refractory pain. According to a further aspect the acute pain is acute pain associated with degenerative joint disease, preferably acute OA-associated pain. According to a further aspect, the chronic pain is chronic pain associated with degenerative joint disease, preferably chronic OA-associated pain. According to a further aspect, the chronic pain is refractory chronic pain associated with degenerative joint disease, preferably refractory chronic OA-associated pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is pain associated with degenerative joint disease, preferably selected from acute pain associated with degenerative joint disease, chronic pain associated with degenerative joint disease, and refractory chronic pain associated with degenerative joint disease.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site; and wherein the NGF-related disorder is OA-associated pain, preferably selected from acute OA-associated pain, chronic OA-associated pain, and refractory chronic OA-associated pain.

The NGF containing CMV VLP compositions were able to booster the neutralising anti-NGF antibodies after repeated or multiple dosing and did not cause any immune tolerance which causes any side effects or lessens the effect of the booster against endogenous NGF protein. This lack of immune-tolerance against the CMV VLP as well as the linked NGF antigen after repeated administration further improves therapeutic benefit in managing a NGF-related disorder, preferably in managing pain.

Thus, according to a further aspect the present invention provides for a composition for use in a method of treating a NGF-related disorder, preferably pain, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site, and wherein the composition is administered to said canine in one or several doses.

In case of repeated dosing, time intervals between the first and the second dose of preferably at least 7 days, preferably at least 14 days, more preferably between 7 and 21 days, more preferably between 14 and 21 days, are beneficial. Further booster administrations are normally provided in longer time intervals. For example, if a third administration is provided, in general a time interval between the second and the third administration of two to six months, for example of three months is chosen. For any further booster administration, a time interval of at least three months, preferably between three to six months, preferably between four and six months is chosen.

For the alleviation of acute pain, in general one or two doses of the NGF containing CMV VLP composition are administered. If two doses are administered, normally a time interval of one to three weeks, preferably between two to three weeks is chosen to obtain a maximum therapeutic effect.

For the alleviation of chronic pain multiple administrations can enhance the therapeutic effect. If three to four doses are administered, normally the time interval between the first and second dose is one to three weeks, preferably between two to three weeks, between the second and third administration two to six months, for example between two to three months and for any fourth administration, an time interval of three to six, preferably between four and six months to the third administration is chosen.

In general, the NGF containing CMV VLP compositions are systemically administered, preferably subcutaneously, intramuscularly or transdermal. In the chosen animal models, subcutaneously administrations have been chosen.

Thus, according to a further aspect the present invention provides for a composition for use in a method of treating a NGF-related disorder, preferably pain, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site, and wherein the composition is administered systemically, preferably subcutaneously, intramuscular or transdermal, preferably subcutaneously.

There are different ways of performing the linkage between the CMV VLP and the NGF antigen. The NGF antigen can be linked to the CMV VLP via the attachment sites by peptide bonds, preferably by genetic fusion. For instance, the nucleotide sequence coding for the NGF antigen can be cloned in frame within the nucleotide sequence coding for the CMV VLP. Alternatively, the NGF antigen can be linked to the CMV VLPs via chemical coupling between the attachment sites, for instance, by at least one non-peptide bond.

Thus, according to a further aspect, the present invention provides for compositions for use in a method of treating an NGF-related disorder, preferably pain, in canine comprising the administration of such composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site via at least one covalent peptide bond by the way of fusion.

According to a further aspect, the present invention provides for a composition for use in a method of treating a NGF-related disorder, preferably pain, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and wherein (a) and (b) are linked through said at least one first and said at least one second attachment site via at least one covalent non-peptide bond.

A CMV VLP is typically a macromolecular assembly composed of viral coat protein which typically comprises 180 protein subunits per VLP. Typically and preferably, the interactions of these subunits lead to the formation of VLPs with an inherent repetitive organization allowing the presentation of multiple copies of NGF antigens. Such coat protein is for example the coat protein as encoded by amino acid sequence SEQ ID NO:39 or having at least 75% sequence identity with SEQ ID NO:39.

Thus, according to a further aspect, the present invention provides for a composition for use in a method of treating a NGF-related disorder, preferably pain, in canine comprising the administration of said composition to said canine, wherein the composition comprises
(a) a VLP of CMV, wherein said VLP of CMV comprises at least one first attachment site, and wherein said VLP of CMV comprises at least one CMV polypeptide, wherein said CMV polypeptide comprises a coat protein of CMV or an amino acid sequence having a sequence identity of at least 75% with SEQ ID NO:39; and
(b) at least one antigen, wherein said antigen comprises at least one second attachment site, and wherein said NGF antigen is a nerve growth factor (NGF) antigen; and
wherein (a) and (b) are linked through said at least one first and said at least one second attachment site. Preferably, said NGF antigen and said VLP of CMV are linked through said at least one first and said at least one second attachment site via at least one covalent non-peptide bond.

The immunogenicity of the NGF containing CMV VLP compositions as described herein can be further increased by adding a T helper (Th) cell epitope. Preferably, said T helper cell epitope is the T helper cell epitope derived from tetanus toxin or is a PADRE sequence. Such T helper cell epitope preferably comprises or consists of amino acid sequence of SEQ ID NO:41 or SEQ ID NO:42. Such T helper cell epitope can be, preferably is, introduced into the CMV VLPs, in particular into the CMV polypeptide and preferably into the coat protein sequence of the CMV VLP. According to a preferred embodiment of these T helper cell epitope containing CMV VLPs, the T helper cell epitope, preferably the T helper cell epitope derived from tetanus toxin or the PADRE sequence, replaces a N-terminal region of the CMV polypeptide. Preferably, the T helper cell epitope, preferably the T helper cell epitope derived from tetanus toxin or the PADRE sequence, replaces a N-terminal region of said CMV polypeptide, and wherein preferably said N-terminal region of said CMV polypeptide correspond to the amino acid residues 2-12 of SEQ ID NO:39.

Thus, according to a further aspect the present invention provides for a composition for use in a method of treating a NGF-related disorder, preferably pain, in canine comprising the administration of said composition to said canine, wherein the composition comprises,
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
(c) a T helper cell epitope; and wherein (a) and (b) are linked through said at least one first and said at least one second attachment site.

According to a further aspect the present invention provides for a composition for use in a method of treating a NGF-related disorder, preferably pain, in canine comprising the administration of said composition to said canine, wherein the composition comprises,
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
(c) a T helper cell epitope, wherein said T helper cell epitope is derived from tetanus toxin or is a PADRE sequence, preferably wherein said T helper cell epitope comprises or consists of amino acid sequence of SEQ ID NO:41 or SEQ ID NO:42; and wherein (a) and (b) are linked through said at least one first and said at least one second attachment site.

According to a further aspect the present invention provides for a composition for use in a method of treating a NGF-related disorder, preferably pain, in canine comprising the administration of said composition to said canine, wherein the composition comprises,
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
(c) a T helper cell epitope, preferably the T helper cell epitope derived from tetanus toxin or the PADRE sequence, preferably comprises or consists of SEQ ID NO:41 or SEQ ID NO:42, replaces a N-terminal region of the CMV polypeptide, preferably, the N-terminal region that correspond to amino acid residues 2-12 of SEQ ID NO:39; and
wherein (a) and (b) are linked through said at least one first and said at least one second attachment site.

In a further aspect the present invention provides for a composition for use in a method of treating a NGF-related disorder, preferably pain, in canine comprising the administration of said composition to said canine, wherein the composition comprises,
(a) a modified VLP of CMV, wherein said modified VLP of CMV comprises at least one first attachment site, and wherein said modified VLP of CMV comprises at least one modified CMV polypeptide, wherein said at least one modified CMV polypeptide comprises, preferably consists of, a CMV polypeptide, wherein said CMV polypeptide comprises a coat protein of CMV or an amino acid sequence having a sequence identity of at least 75% with SEQ ID NO:39; and
(b) at least one antigen, wherein said antigen comprises at least one second attachment site, and wherein said NGF antigen is a nerve growth factor (NGF) antigen; and
wherein (a) and (b) are linked through said at least one first and said at least one second attachment site. Preferably, said NGF antigen and said modified VLP of CMV are linked through said at least one first and said at least one second attachment site via at least one covalent non-peptide bond. In a preferred embodiment, said modified CMV polypeptide comprises, preferably consists of, the amino acid sequence of SEQ ID NO:5 or of SEQ ID NO: 48.

In addition, or alternatively to the immunogenic properties, the physico-chemical properties of the CMV VLPs could further be improved by the insertions a stretch of consecutive negative amino acids, wherein said negative amino acids are independently selected from aspartic acid or glutamic acid. Such CMV VLPs showed improved stability under elevated temperatures and higher ionic strengths. Moreover, when conjugated to NGF antigen, such CMV VLP-NGF antigen conjugates did not form aggregates and remained stable in solution upon linking the NGF antigen, while prior art CMV VLPs formed certain amounts of aggregates and precipitates. Such improved (non-aggregated) CMV VLP-NGF antigen conjugates are highly desired for drug development and product registration.

Thus, according to a further aspect, the present invention provides for a composition for use in a method of treating an NGF-related disorder, preferably pain, in canine comprising the administration of said composition to said canine, wherein said composition comprises,
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, wherein said polypeptide is inserted into the CMV polypeptide of (i);
(b) at least one antigen, wherein said antigen comprises at least one second attachment site, and wherein said antigen is a nerve growth factor (NGF) antigen; and
wherein (a) and (b) are linked through said at least one first and said at least one second attachment site, preferably via at least one covalent non-peptide bond.

Preferably said stretch of consecutive negative amino acids independently selected from aspartic acid or glutamic acid, is inserted between any amino acid residue of said CMV polypeptide corresponding to any amino acid residue between position 75 and position 85 of SEQ ID NO:39.

The stretch of consecutive negative amino acids normally has a length of 3 to 10 amino acids, and according to a further aspect may consists solely of glutamic acids.

Thus, according to a further aspect, the present invention provides for a composition for use in a method of treating an NGF-related disorder, preferably pain, in canine comprising the administration of said composition to said canine, wherein said composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, preferably between 3 and 10 amino acid residues, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:39.
(b) at least one antigen, wherein said antigen comprises at least one second attachment site, and wherein said antigen is nerve growth factor (NGF) antigen; and
wherein (a) and (b) are linked through said at least one first and said at least one second attachment site, preferably via at least one covalent non-peptide bond. Preferably, said chimeric CMV polypeptide comprises, preferably consists of, the amino acid sequence of SEQ ID NO:10, SEQ ID NO:11 or SEQ ID NO:12.

According to a further aspect, the stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of:
i) a polyglycine linker (G-linker) having an amino acid sequence (Gly)ₙ of a length of n=2-10;
ii) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and
iii) an amino acid linker (GS*-linker) comprising at least one Gly, at least one Ser, and at least one amino acid selected from Thr, Ala, Lys, and Cys.

According to a further aspect the polypeptide comprising the stretch of consecutive negative amino acids consists of SEQ ID NO:49, SEQ ID NO:50 or SEQ ID NO:51.

According to a further aspect the present invention provides for a composition for use in a method of treating a NGF-related disorder, preferably pain, in canine comprising the administration of said composition to said canine, wherein the composition comprises,
(a) a virus-like particle (VLP) of Cucumber Mosaic Virus (CMV), wherein said CMV VLP comprises at least one first attachment site and further comprises at least one T helper cell epitope and, wherein such VLP of CMV comprises, preferably consists of, the amino acid sequence of SEQ ID NO:10, SEQ ID NO: 11 or SEQ ID NO:12; and
(b) at least one antigen, wherein said antigen comprises at least one second attachment site, and wherein said antigen is a nerve growth factor (NGF) antigen; and and
wherein (a) and (b) are linked through said at least one first and said at least one second attachment site, preferably by a non-peptide bond.

The NGF antigen to be used in any of the NGF-containing CMV VLP compositions for use in a method of treating a NGF-related disorder in a canine as described herein can be any NGF antigen that shows the desired technical effect in canine. Preferably that NGF antigen is canine (cNGF). According to a further aspect the NGF antigen to be used in the NGF-containing CMV VLP compositions for use in a method of treating a NGF-related disorder in canine according to the invention comprises, or preferably consists of, an amino acid sequence selected from any of SEQ ID NO:30, SEQ ID NO:31, SEQ ID NO:33, SEQ ID NO: 54, SEQ ID NO:55, SEQ ID NO:56, SEQ ID NO:57, and SEQ ID NO:58, or an amino acid sequence having a sequence identity of at least 90%, preferably of at least 95%, with any of SEQ ID NO:30, SEQ ID NO:31, SEQ ID NO:33, SEQ ID NO:54, SEQ ID NO: 55, SEQ ID NO:56, SEQ ID NO:57, and SEQ ID NO:58.

According to a further aspect the NGF antigen to be used in the NGF-containing CMV VLP compositions for use in a method of treating a NGF-related disorder in canine according to the invention comprises, or preferably consists of, an amino acid sequence selected from any of SEQ ID NO:30, SEQ ID NO:31, SEQ ID NO:33 and SEQ ID NO:55, or an amino acid sequence having a sequence identity of at least 90%, preferably of at least 95%, with any of SEQ ID NO:30, SEQ ID NO:31, SEQ ID NO:33 and SEQ ID NO:55.

### BRIEF DESCRIPTION OF FIGURES

FIG. 1: Description of pET-CMVB2-Ntt-E8* plasmid map with single-cut restriction enzyme sites.

FIG. 2A: SDS-PAGE gel analysis of the purification of the VLP derived from the expression of CMV-Ntt830-E8*. M—protein size marker PageRuler (Thermo Fisher Scientific, #26620); S—soluble proteins in cell extract in E. coli C2566/pET-CMVB2-Ntt-E8*; P—insoluble proteins in cell extract; 1—insoluble proteins after sucrose gradient (bottom of the tube); 2-6—sucrose gradient fractions (from 60% at the bottom of tube to 0% at the top). The asterisk (*) within the figure denotes the relative position of the corresponding CMV-Ntt830-E8* chimeric CMV polypeptide in SDS/PAGE gel.

FIG. 2B: Electron microscopy images of purified CMV-Ntt830-E8* VLPs. The horizontal bar corresponds to 500 nm.

FIG. 3: Description of pET-CMVB2-Ntt-E4 plasmid map with single-cut restriction enzyme sites.

FIG. 4: Description of pET-CMVB2-Ntt-E8 plasmid map with single-cut restriction enzyme sites.

FIG. 5: Description of pET-CMVB2-Ntt-E12 plasmid map with single-cut restriction enzyme sites.

FIG. 6: SDS-PAGE (left) and agarose gel (right) analysis of the purification of the VLP derived from the expression of CMV-Ntt830-E4. M1-protein size marker PageRuler (Thermo Fisher Scientific, #26620); M2-DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E4 chimeric CMV polypeptide in SDS/PAGE gel and typical VLP signal in agarose gel.

FIG. 7: SDS-PAGE (left) and agarose gel (right) analysis of the purification of the VLP derived from the expression of CMV-Ntt830-E8. M1-protein size marker PageRuler (Thermo Fisher Scientific, #26620); M2-DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E8 chimeric CMV polypeptide in SDS/PAGE gel and typical VLP signal in agarose gel.

FIG. 8: SDS-PAGE (left) and agarose gel (right) analysis of the purification of the VLPs derived from the expression of CMV-Ntt830-E12. M1-protein size marker PageRuler (Thermo Fisher Scientific, #26620); M2-DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E12 chimeric CMV polypeptide in SDS/PAGE gel. A clear and distinct band corresponding to intact VLPs was not observed in the agarose gel.

FIG. 9: Electron microscopy images of purified CMV-Ntt830-E4 VLPs. Horizontal bar corresponds to 200 nm.

FIG. 10: Electron microscopy images of purified CMV-Ntt830-E8 VLPs. Horizontal bar corresponds to 200 nm.

FIG. 11: Comparison of thermal stability of CMV-Ntt830 VLPs and CMV-Ntt830-E4 VLPs. The structural changes in CMV-Ntt830 VLPs and CMV-Ntt830-E4 VLPs were monitored in the presence of Sypro-Orange dye using a DNA melting point determination program and a real-time PCR system. Curve 1 is for CMV-Ntt830-E4 VLPs), curve 2 is for CMV-Ntt830 VLPs and Curve 3 is for buffer control (5 mM Na phosphate 2 mM EDTA, pH 7.5). The respective 57° C. and 51° C. melting points are indicated by arrows.

FIG. 12: Stability of CMV-Ntt830 VLPs and CMV-Ntt830-E4 VLPs in solution in the presence of different NaCl concentrations. Samples of CMV-Ntt830 VLPs and CMV-Ntt830-E4 VLPs at 0.5 mg/ml were incubated at room temperature in 5 mM Na phosphate, 2 mM EDTA, pH 7.5 with different concentrations of NaCl (the molar concentration of NaCl in each sample is indicated at the bottom of the gels) for up to 2 hours. Samples were analysed by native agarose gel electrophoresis and ethidium bromide staining. Panels A and B show NAGE analysis of CMV-Ntt830 VLP and CMV-Ntt830-E4 VLPsamples respectively. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis (panel A) is due to the formation of VLP aggregates which are too large to enter the gel. Integral unaggreagted VLPs migrated into the gel.

FIG. 13: Analysis of CMV-Ntt830 VLPs subject to Anion Exchange Chromatography. 5 ml of 1 mg/ml CVMtt-VLPs in 5 mM Sodium Borate buffer pH 9.0 was loaded onto 1.0 ml Macro-Prep DEAE Bio-Rad anion exchange cartridge equilibrated with 5 mM Sodium Borate buffer and eluted step-wise with increasing concentrations of NaCl (0.1, 0.2, 0.3, 0.4, 0.5, 0.8, 1.0 and 2.0 M). Fractions were collected and analysed by nanodrop 260 nm for protein concertation and native agarose gel electrophoresis. Panel A shows the NaCl concentration and 260 nm absorbance values plotted against the respective fractions (1-25). Panel B is a NAGE analysis (ethidium bromide stained) of the principle fractions containing the highest protein concentrations. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis is due to the formation of VLP aggregates which are too large to enter the gel. Integral unaggreagted VLPs migrated into the gel.

FIG. 14: Analysis of CMV-Ntt830-E4 VLPs subject to Anion Exchange Chromatography. A biomass of E. coli cells expressing CMV-Ntt830-E4 VLPs was resuspended in 50 mM citrate, 5 mM Borate buffer pH 9.0 and cells were lysed using a microfluidizer LM-20. The soluble fraction was clarified by centrifugation and loaded onto a 60 ml Fracto-DEAE (XK 26/20). An elution buffer comprising 50 mM Citrate 5 mM Borate and 1M NaCl was applied in a continuous gradient manner to elute the bound VLPs. Panel A shows the protein elution and NaCl concentration gradient measured by A260 nm (mAU) and conductivity (mS/cm) respectively. The X-axis shows the elution volume and fraction numbers (4-11). The fractions collected from the Fracto-DEAE column were analysed by NAGE (panel B) and SDS-PAGE (panel C). In panel B, M indicates the lane loaded with a GeneRuler 1 kb DNA Ladder (SM0311, TFS), L is a sample of E. coli lysate before loading onto the Fracto DEAE, FT is the flow through collected from 0 to 150 ml and 4-10 represent the fraction numbers collected during elution. The black arrows from top to bottom indicate the position of the loading wells, position of integral CMV-Ntt830-E4 VLPs within the gel and contaminating nucleic acids from the clarified bacterial lysate respectively. In panel C, FT is the flow through collected from 0 to 150 ml and 4-10 represent the fraction numbers. The black arrow shows the position of the Coomassie blue stained CMV-Ntt830-E4 coat protein.

FIG. 15A: Purification and authenticity of recombinant canine mature NGF. SDS-PAGE analysis of the NGF purification process. M—marker, with molecular weights of bands shown in kDa; A—total cell lysate after expression, B—pooled fractions containing pro-NGF after refolding and partial purification; C—mature NGF after trypsin digestion and final purification. Arrows indicate pro-NGF in lanes A and B and mature NGF in lane C.

FIG. 15B: PC12 cells were grown for 5 days with recombinant human mature NGF produced in mouse myeloma cells (R&D systems) (black squares) or with canine mature NGF produced in E. coli as described herein (grey circles). Cells were grown in the presence of 100, 50, 25, 12.5 and 6.25 ng/ml of recombinant NGF and the percentage of cells with defined neurite outgrowth determined.

FIG. 16A: SDS-PAGE analysis of coupling of recombinant mature canine NGF (cNGF) of Seq ID NO:31 to CMV-Ntt830 and CMV-Ntt830-E8* VLPs.

M—PageRuler™ Plus Prestained Protein Ladder, 10 to 250 kDa (Thermo Fisher Scientific, #26620) protein size marker; 1—Corresponding purified CMV-Ntt830 and CMV-Ntt830-E8* VLPs; 2—CMV VLPs after derivatization with 5×SMPH and removal of SMPH; 3—CMV VLPs coupled with equimolar amount of cNGF; 4—mixed samples of CMV-Ntt830-E8* and cNGF without SMPH derivatization; 5—purified cNGF. The asterixis denote the localization of observable CMV VLPs-NGF conjugate bands.

FIG. 16B: SDS-PAGE analysis of coupling of recombinant mature canine NGF (cNGF) of SEQ ID NO:31 to CMV-Ntt830-E4 and CMV-Ntt830-E8 VLPs. M—PageRuler™ Plus Prestained Protein Ladder, 10 to 250 kDa (Thermo Fisher Scientific, #26620) protein size marker; 1—Corresponding purified CMV-Ntt830-E4 and CMV-Ntt830-E8 VLPs; 2-CMV VLPs after derivatization with 5×SMPH and removal of SMPH; 3—CMV VLPs coupled with equimolar amount of cNGF; 4—mixed samples of CMV-Ntt830-E4 or CMV-Ntt830-E8 and cNGF without SMPH derivatization; 5—purified cNGF. The asterixis denote the localization of observable CMV VLPs-cNGF conjugate bands.

FIG. 16C: Dynamic light scattering analysis of cNGF-CMV-Ntt830 VLPs. Because the vaccine precipitated, EM analysis could not be performed.

FIG. 16D: Dynamic light scattering analysis of cNGF-CMV-Ntt830-E4 VLPs comprising cNGF antigens of SEQ ID NO:31.

FIG. 16E: Dynamic light scattering analysis of cNGF-CMV-Ntt830-E4 VLPs comprising cNGF antigens of SEQ ID NO:33.

FIG. 16F: Dynamic light scattering analysis of cNGF-CMV-Ntt830-E8* VLPs

FIG. 16G: Electromicroscopy of cNGF-CMV-Ntt830-E4 VLPs.

FIG. 16H: Electromicroscopy of cNGF-CMV-Ntt830-E8* VLPs.

FIG. 17A: Assessment of anti-NGF IgG antibodies from sera of dogs immunized with cNGF-CMV-Ntt830-E8* VLP. Anti-NGF IgG titers of dogs from group 1 that received vaccine without adjuvant. Arrows indicate the injections of vaccine administered on day 0, 21 and 42.

FIG. 17B: Assessment of anti-NGF IgG antibodies from sera of dogs immunized with cNGF-CMV-Ntt830-E8* VLP. Anti-NGF IgG titers of 3 dogs from group 2 that received vaccine with adjuvant QuilA®. Arrows indicate the injections of vaccine administered on day 0, 21 and 42.

FIG. 17C: Assessment of anti-CMV IgG titers from sera of dogs immunized with cNGF-CMV-Ntt830-E8*VLP. Anti-CMV IgG titers of dogs from group 1 that received vaccine without adjuvant. Arrows indicate the injections of vaccine administered on day 0, 21 and 42.

FIG. 17D: Assessment of anti-CMV IgG titers from sera of dogs immunized with cNGF-CMV-Ntt830-E8* VLP. Anti-CMV IgG titers of dogs from group 2 that received vaccine with adjuvant QuilA®. Arrows indicate the injections of vaccine administered on day 0, 21 and 42.

FIG. 17E: Assessment of anti-NGF IgG antibodies from sera of dogs immunized with cNGF-CMV-Ntt830-E4 VLP in absence of adjuvant. 5 dogs were dosed with cNGF-CMV-Ntt830-E4 VLP on day 0 and 21. NGF-specific antibodies were assessed by ELISA in serum collected on days 0, 21, 42, 71 and 91.

FIG. 17F: Assessment of anti-NGF IgG antibodies from sera of dogs immunized with cNGF-CMV-Ntt830-E4 VLP in presence of aluminum hydroxide. 5 dogs were dosed with cNGF-CMV-Ntt830-E4 VLP with aluminum hydroxide on day 0 and 21. NGF-specific antibodies were determined by ELISA on days 0, 21, 42, 71 and 91.

FIG. 18A: Vaccination with cNGF-CMV-Ntt830-E8* VLP induces NGF neutralizing antibodies in dogs. Dogs (3 dogs per group) were immunized with 250 μg cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. Representation of titration of curves from one dog to determine neutralization capacity and 50% neutralization titers (NT50) of dog sera. 5 ng/mL human mature NGF was preincubated with increasing concentration of IgG purified from sera collected at indicated days after first administration of the vaccine. NT50 values, i.e. IgG concentration leading to 50% inhibition of cell proliferation, were determined using a 4PL sigmoidal curve fit model.

FIG. 18B: Vaccination with cNGF-CMV-Ntt830-E8* VLP induces NGF neutralizing antibodies in dogs. Dogs (3 dogs per group) were immunized with 250 μg cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. Total IgG were purified from dog sera. The capacity of 20 μg/mL of purified total IgG to neutralize 5 ng human mature NGF/mL was assessed using the bioassay. Bars represent mean group values with standard deviation and symbols represent individual dogs (mean of assay duplicate). 2-way ANOVA with Tukey's multiple comparisons test was performed to compare group mean values using GraphPad Prism. *p<0.05, **p<0.01, ***p<0.001, ****p<0.0001.

FIG. 18C: Vaccination with cNGF-CMV-Ntt830-E8* VLP induces mature NGF neutralizing antibodies in dogs. Dogs (3 dogs per group) were immunized with 250 μg cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. NT50 values were plotted versus OD50 values of anti-NGF IgG serum titers. Total IgG purified from serum with higher concentrations of NGF-specific antibodies were more potent to inhibit NGF mediated TF-1 cell proliferation than total IgG purified from sera of dogs with lower anti-NGF titers. Symbols represent individual dogs and sampling time points. Different symbols were allocated to different dogs. Closed symbols represent animals vaccinated in presence of adjuvant, whereas open symbols representing animals vaccinated without adjuvant.

FIG. 18D: Vaccination with cNGF-CMV-Ntt830-E4 VLP induces NGF neutralizing antibodies in dogs. cNGF-CMV-Ntt830-E4 VLP with aluminum hydroxide was adminstered to 5 dogs on day 0 and 21. Sera collected on day 42 were tested for presence of neutralizing antibodies using a TF-1 based NGF bioactivity assay. Bars represent mean group values with standard deviation and symbols represent individual dogs. The dotted line indicates detection limit of the assay.

FIG. 19A: Kaolin model of acute inflammatory pain: Vaccination of dogs with cNGF-CMV-Ntt830-E4 VLPs (squares) as compared to treatment with prior art monoclonal antibody, bedinvetmab (diamond), and placebo (triangles)-cNGF specific IgG titers (GMTs) of the study groups were graphed as EC 50 values. The error bars represent the 95% CI of the GMT. Assay results below the LoD were set to 50, corresponding to 0.5× the lowest dilution factor (i.e., 1:100) used in the assays (dotted line). GMT equal geometric mean titer; LoD=limit of detection; LoQ=limit of quantitation.

FIG. 19B: Kaolin model of acute inflammatory pain: Vaccination of dogs with cNGF-CMV-Ntt830-E4 VLPs (squares) as compared to treatment with prior art monoclonal antibody, bedinvetmab (triangles), and placebo (circles)-cNGF specific IgG titers (GMTs) of the study groups were graphed as standard equivalent concentration. The error bars represent the 95% CI of the GMT. Titers above the LoD but below the LoQ were set to 100. Peak antibody titer in serum after administration of bedinvetmab has previously been reported as 6.1 μg/mL (dashed line). GMT equal geometric mean titer; LoD=limit of detection; LoQ=limit of quantitation.

FIG. 20A: Kaolin model of acute inflammatory pain: Vaccination of dogs with cNGF-CMV-Ntt830-E4 VLPs (squares) as compared to treatment with prior art monoclonal antibody, bedinvetmab (triangles), and placebo (circles)—NGF neutralization titers determined by the TrkA based NGF binding ELISA. NGF neutralization GMTs of the study groups were graphed as EC 50 values. The error bars represent the 95% CI of the GMT. Assay results below the LoD were set to 50, corresponding to 0.5× the lowest dilution factor (i.e., 1:100) used in the assays (dotted line). GMT equal geometric mean titer; LoD=limit of detection; LoQ=limit of quantitation.

FIG. 20B: Kaolin model of acute inflammatory pain: Vaccination of dogs with cNGF-CMV-Ntt830-E4 VLPs (squares) as compared to treatment with prior art monoclonal antibody, bedinvetmab (triangles), and placebo (circles)—NGF neutralization titers determined by the TrkA based NGF binding ELISA. NGF neutralization GMTs of the study groups were graphed as standard equivalent concentration. The error bars represent the 95% CI of the GMT. Titers above the LoD but below the LoQ were set to 100. Peak antibody titer in serum after administration of bedinvetmab has previously been reported as 6.1 μg/mL (dashed line). GMT equal geometric mean titer; LoD=limit of detection; LoQ=limit of quantitation.

FIG. 21A: Kaolin model of acute inflammatory pain: Vaccination of dogs with cNGF-CMV-Ntt830-E4 VLPs (squares) as compared to treatment with prior art monoclonal antibody, bedinvetmab (triangles), and placebo (circles)—NGF neutralization GMTs determined by the TF-1 cell-based NGF bioassay. NGF neutralization GMTs of the study groups were graphed as EC 50 values. The error bars represent the 95% CI of the GMT. Assay results below the LoD were set to 50, corresponding to 0.5× the lowest dilution factor (i.e., 1:100) used in the assays (dotted line). GMT equal geometric mean titer; LoD=limit of detection; LoQ=limit of quantitation.

FIG. 21B: Kaolin model of acute inflammatory pain: Vaccination of dogs with cNGF-CMV-Ntt830-E4 VLPs (squares) as compared to treatment with prior art monoclonal antibody, bedinvetmab (triangles), and placebo (circles)—NGF neutralization GMTs determined by the TF-1 cell-based NGF bioassay. NGF neutralization GMTs of the study groups were graphed as standard equivalent concentration. The error bars represent the 95% CI of the GMT. Titers above the LoD but below the LoQ were set to 100. Peak antibody titer in serum after administration of bedinvetmab has previously been reported as 6.1 μg/mL (dashed line). GMT equal geometric mean titer; LoD=limit of detection; LoQ=limit of quantitation.

FIG. 22: Surgical model of acute pain: 4 groups of 7 dogs were treated with, respectively, the cNGF-CMV-Ntt830-E4 VLP vaccine, monoclonal bedinvetmab (Librela™), meloxicam (Inflacam™) and placebo (water for injection). Treatments were implemented, respectively, 21 and 42 days, and 7 days and 21 and 42 days before implementation of an inflammatory/acute pain experimental model resulting from the injection of kaolin chemical into the right hind paw of all dogs. Lameness score was assessed on a typical course made by each dog using a validated numerical rating scale. The numerical rating scale for the evaluation of lameness in the inflamed paw was based on the examiner's perception and the score was established as follows: (0) No lameness; (1) Barely detectable lameness over most of the observation period; (2) Mild lameness, substantial weight bearing; (3) Moderate lameness, minimal weight bearing; (4) Severe lameness, the animal uses his paw (walking movement initiated and/or touches lightly the ground) but does not bear weight; (5) The animal could not be more lame, refuse to move and/or avoid any contact of the inflamed paw with the ground. Lameness score was measured on all dogs over a period of 2 weeks after the kaolin challenge.

FIG. 23: The Area Under the Curve (AUC) for lameness scores of dogs treated with, respectively, the cNGF-CMV-Ntt830-E4 VLP vaccine, monoclonal bedinvetmab (Librela™), meloxicam (Inflacam™) and placebo (water for injection) was calculated over a period of 14 days post kaolin challenge.

FIG. 24A: Surgical experimental model of chronic pain: Vaccination of dogs with cNGF-CMV-Ntt830-E4 VLPs (Group A)—cNGF specific IgG titers (GMTs) of the study groups were graphed as EC 50 values. The error bars represent the 95% CI of the GMT. Assay results below the LoD were set to 50, corresponding to 0.5× the lowest dilution factor (i.e., 1:100) used in the assays (dotted line). GMT equal geometric mean titer; LoD=limit of detection; LoQ=limit of quantitation.

FIG. 24B: Surgical experimental model of chronic pain: Vaccination of dogs with cNGF-CMV-Ntt830-E4 VLPs (Group B)—cNGF specific IgG titers (GMTs) of the study groups were graphed as standard equivalent concentration. The error bars represent the 95% CI of the GMT. Titers above the LoD but below the LoQ were set to 100. Peak antibody titer in serum after administration of bedinvetmab has previously been reported as 6.1 μg/mL (dashed line). GMT equal geometric mean titer; LoD=limit of detection; LoQ=limit of quantitation.

FIG. 25A: Geometric mean NGF neutralization titers determined by the TrkA based NGF binding ELISA. NGF neutralization GMTs of study group were graphed as EC50 value. The error bars represented the 95% CI of GMT. Assay results below the LoD were set to 50, corresponding to 0.5× the lowest dilution factor (i.e., 1:100) used in the assay (dotted line). Titers above LoD but below the LoQ were set 100. GMT equal geometric mean titer; LoD=limit of detection; LoQ=limit of quantitation.

FIG. 25B: Geometric mean NGF neutralization titers determined by the TrkA based NGF binding ELISA. NGF neutralization GMTs of study group were graphed as standard equivalent concentration. The error bars represented the 95% CI of GMT. Peak antibody titer in serum after administration of bedinvetmab was reported as 6.1 μg/ml (dashed line). GMT geometric mean titer; LoD=limit of detection; LoQ=limit of quantitation.

FIG. 26A: NGF neutralization GMTs determined by the TF-1 cell based NGF bioassay. NGF neutralization GMTs of study group were graphed as EC50 value. The error bars represented the 95% CI of GMT. Assay results below the LoD were set to 50, corresponding to 0.5× the lowest dilution factor (i.e., 1:100) used in the assay (dotted line). Titers above LoD but below the LoQ were set 100. GMT equal geometric mean titer; LoD=limit of detection; LoQ=limit of quantitation.

FIG. 26B: NGF neutralization GMTs determined by the TF-1 cell based NGF bioassay. NGF neutralization GMTs of study group were graphed as standard equivalent concentration. The error bars represented the 95% CI of GMT. Peak antibody titer in serum after administration of bedinvetmab was reported as 6.1 μg/ml (dashed line). GMT geometric mean titer; LoD=limit of detection; LoQ=limit of quantitation.

FIG. 27A: Surgical model of chronic pain: 2 groups of 7 dogs were treated with, respectively, the cNGF-CMV-Ntt830-E4 VLP vaccine and placebo (water for injection). Treatments were implemented both at, approximatively, 21 and 42 days before implementation of an chronic pain experimental model resulting from the destabilisation of the right stiffle joints by surgery.

Local pain at the treated joints was assessed by palpation and scored according to a pre-defined scale of Pain Palpation grading (ranging from 0 to 4) derived from the Modified Glasgow Composite Pain Scale. Reaction to palpation (alodynia) scores were measured on all dogs at pre-defined timepoints from study day 84 (SD84) to SD217.

FIG. 27B: Surgical experimental model of chronic pain: Vaccination of dogs with cNGF-CMV-Ntt830-E4 VLPs (Group B)—cNGF specific IgG titers (GMTs) of the study groups were graphed as standard equivalent concentration. The error bars represent the 95% CI of the GMT.

FIG. 27C: Surgical model of chronic pain: 2 groups of 7 dogs were treated with, respectively, the cNGF-CMV-Ntt830-E4 VLP vaccine and placebo (water for injection). Treatments were implemented both at, approximatively, 21 and 42 days before implementation of an chronic pain experimental model resulting from the destabilisation of the right stiffle joints by surgery. Lameness score was assessed on a typical course made by each dog using a validated numerical rating scale. The numerical rating scale for the evaluation of lameness was based on the examiner's perception and the score was established as follows: (0) No lameness; (1) Barely detectable lameness over most of the observation period; (2) Mild lameness, substantial weight bearing; (3) Moderate lameness, minimal weight bearing; (4) Severe lameness, the animal uses his paw (walking movement initiated and/or touches lightly the ground) but does not bear weight; (5) The animal could not be more lame, refuse to move and/or avoid any contact of the inflamed paw with the ground. Lameness score was measured on all dogs at study day 217 i.e., approximatively 175 days after surgery.

FIG. 28A: Pain assessment comparisons between groups. Groups-A: vaccine candidate, B: bedinvetmab, C: no treatment, at SD54-SD59 (at surgery), SD84, SD133, SD182, SD203 and SD217. Kruskal-Wallis statistical analysis with Dunn's post-test.

FIG. 28B: Lameness assessment comparisons between groups. Groups-A: vaccine candidate, B: bedinvetmab, C: no treatment, at SD54-SD59 (at surgery), SD84, SD133, SD182, SD203 and SD217. Kruskal-Wallis statistical analysis with Dunn's post-test.

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and/or very preferred embodiments should apply to all aspects and other embodiments, preferred embodiments and/or very preferred embodiments irrespective of whether is specifically again referred to or irrespective of whether its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA. A typical and preferred embodiment of a virus-like particle in accordance with the present invention is a viral capsid composed of polypeptides of the invention. A virus-like particle is typically a macromolecular assembly composed of viral coat protein which typically comprises 60, 120, 180, 240, 300, 360, or more than 360 protein subunits per virus-like particle. Typically and preferably, the interactions of these subunits lead to the formation of viral capsid or viral-capsid like structure with an inherent repetitive organization. One feature of a virus-like particle is its highly ordered and repetitive arrangement of its subunits.

Virus-like particle of Cucumber Mosaic Virus (CMV): The terms “virus-like particle of CMV” or “CMV VLPs” refer to a virus-like particle comprising, or preferably consisting essentially of, or preferably consisting of at least one CMV polypeptide. Preferably, a virus-like particle of CMV comprises said CMV polypeptide as the major, and even more preferably as the sole protein component of the capsid structure. Typically and preferably, virus-like particles of CMV resemble the structure of the capsid of CMV. Virus-like particles of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also virus-like particles in which the aforementioned gene or genes are still present but inactive. Preferred methods to render a virus-like particle of CMV non replicative and/or non-infectious is by physical or chemical inactivation, such as UV irradiation, formaldehyde treatment. Preferably, VLPs of CMV lack the gene or genes encoding for the replication machinery of the CMV, and also lack the gene or genes encoding the protein or proteins responsible for viral attachment to or entry into the host. Again more preferably, non-replicative and/or non-infectious virus-like particles are obtained by recombinant gene technology. Recombinantly produced virus-like particles of CMV according to the invention typically and preferably do not comprise the viral genome. Preferably, a VLP of CMV is a macromolecular assembly composed of CMV coat proteins or CMV polypeptides which typically comprises 180 coat protein or CMV polypeptide subunits per VLP. Typically and preferably, a VLP of CMV as used herein, comprises, essentially consists of, or alternatively consists of, at least one CMV polypeptide comprising or preferably consisting of (i) an amino acid sequence of a coat protein of CMV; or (ii) a mutated amino acid sequence, wherein said mutated amino acid sequence has a sequence identity of at least 90%, preferably of at least 95%, further preferably of at least 98% and again more preferably of at least 99% to said coat protein of CMV.

Modified virus-like particle (VLP) of CMV: The term “modified virus-like particle of CMV” refers to a virus-like particle comprising at least one modified CMV polypeptide, at least one chimeric CMV polypeptide, or at least one antigenic CMV fusion polypeptide, as defined and as described herein. Typically and preferably, modified VLPs of CMV resemble the structure of the capsid of CMV. Modified VLPs of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also modified virus-like particles in which the aforementioned gene or genes are still present but inactive. Preferably, non-replicative and/or non-infectious modified virus-like particles are obtained by recombinant gene technology and typically and preferably do not comprise the viral genome. Preferably, a modified VLP of CMV is a macromolecular assembly composed of CMV polypeptides modified as described herein, and typically and preferably comprising 180 of such protein subunits including modified CMV polypeptides, chimeric polypeptides or antigenic CMV fusion polypeptides, respectively per VLP. Thus, in a preferred embodiment, said modified VLP of CMV comprises, preferably consists of, 180 modified CMV polypeptides. In another preferred embodiment, said modified VLP of CMV comprises, preferably consists of, 180 chimeric CMV polypeptides. In a further preferred embodiment, said modified VLP of CMV is mosaic fusion CMV VLP comprising at least one, preferably a plurality of, antigenic CMV fusion polypeptides.

Mosaic fusion CMV VLP: The term “mosaic fusion CMV VLP” refers to a modified VLP of CMV as defined herein and comprising at least one, preferably a plurality of, antigenic CMV fusion polypeptides, as defined herein. Virus-like particles comprising more than one species and kind of polypeptides are referred to as mosaic VLPs. Thus, in a further preferred embodiment, said composition comprises, preferably consists of, said mosaic fusion CMV VLP, wherein said mosaic fusion CMV VLP comprises at least one, preferably a plurality of, antigenic CMV fusion polypeptides, as defined herein, and further comprises polypeptides selected from CMV polypeptides, modified CMV polypeptides, and CMV proteins, all as defined herein.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. The term “polypeptide” as used herein should also refer, typically and preferably to a polypeptide as defined before and encompassing modifications such as post-translational modifications, including but not limited to glycosylations. In a preferred embodiment, said term “polypeptide” as used herein should refer to a polypeptide as defined before and not encompassing modifications such as post-translational modifications such as glycosylations. In particular, for said biologically active peptides, said modifications such as said glycosylations can occur even in vivo thereafter, for example, by bacteria.

Cucumber Mosaic Virus (CMV) polypeptide, CMV polypeptide: The term “cucumber mosaic virus (CMV) polypeptide” as used herein refers to a polypeptide comprising or preferably consisting of: (i) an amino acid sequence of a coat protein of cucumber mosaic virus (CMV), or (ii) a mutated amino acid sequence, wherein said mutated amino acid sequence and said coat protein of CMV show a sequence identity of at least 90%, preferably of at least 91%, 92%, 93% or 94%, further preferably of at least 95%, again further preferably of at least 98% and further more preferably of at least 99%. Typically and preferably, the CMV polypeptide is capable of forming a virus-like particle of CMV upon expression by self-assembly.

Coat protein (CP) of cucumber mosaic virus (CMV): The term “coat protein (CP) of cucumber mosaic virus (CMV)”, as used herein, refers to a coat protein of the cucumber mosaic virus which occurs in nature. Due to extremely wide host range of the cucumber mosaic virus, a lot of different strains and isolates of CMV are known. The sequences of the coat proteins of said strains and isolates have been determined and are known to the skilled person in the art. The sequences of said coat proteins (CPs) of CMV are described in and retrievable from the known databases such as Genbank, www.dpvweb.net, or www.ncbi.nlm.nih.gov/protein/. Specific examples CPs of CMV are described in WO 2016/062720 at page 12, line 8 to page 13, line 25, the disclosure of which are explicitly incorporated herein by way of reference. A very preferred example and embodiment of a CMV coat protein is provided in SEQ ID NO:39. Thus, preferably, the term “coat protein of cucumber mosaic virus (CMV)”, as used herein, refers to an amino acid sequence of a coat protein of CMV, wherein said amino acid sequence comprises, or preferably consists of, SEQ ID NO:39 or an amino acid sequence having a sequence identity of at least 75%, preferably of at least 80%, more preferably of at least 85%, again further preferably of at least 90%, again further preferably of at least 91%, 92%, 93% or 94%, again more preferably of at least 95%, still further preferably of at least 96% or 97%, still further preferably of at least 98% and still again further more preferably of at least 99% of SEQ ID NO:39.

It is noteworthy that these strains and isolates have highly similar coat protein sequences at different protein domains, including the N-terminus of the coat protein. In particular, 98.1% of all completely sequenced CMV isolates share more than 85% sequence identity within the first 28 amino acids of their coat protein sequence, and still 79.5% of all completely sequenced CMV isolates share more than 90% sequence identity within the first 28 amino acids of their coat protein sequence.

Modified CMV polypeptide: The term “modified CMV polypeptide” as used herein refers to a CMV polypeptide comprising, or preferably consisting of, a CMV polypeptide, and a T helper cell epitope. Typically, the modified CMV polypeptide is capable of forming a virus-like particle of CMV upon expression by self-assembly. Preferably, the modified CMV polypeptide is a recombinant modified CMV polypeptide and is capable of forming a virus-like particle of CMV upon expression by self-assembly in E. coli.

Chimeric CMV polypeptide: The term “chimeric CMV polypeptide” as used herein refers to a polypeptide as defined herein and in accordance with the present invention, and comprising, preferably consisting of, a CMV polypeptide, wherein said CMV polypeptide is modified as defined and described herein, to comprise a polypeptide comprising, preferably consisting of, a stretch of consecutive negative amino acids independently selected from aspartic acid or glutamic acid, and optionally further to comprise a T helper cell epitope, all components as defined and described herein. Typically and preferably, the chimeric CMV polypeptide is capable of forming a modified virus-like particle of CMV upon expression by self-assembly. Thus, in a preferred embodiment, said chimeric CMV polypeptide is capable of forming a modified virus-like particle of CMV by self-assembly, typically and preferably by self-assembly upon expression. Preferably, the chimeric CMV polypeptide is a recombinant modified CMV polypeptide and is capable of forming a virus-like particle of CMV upon expression by self-assembly in E. coli. Typically and preferably, said T helper cell epitope replaces a N-terminal region of said CMV polypeptide, wherein said replaced N-terminal region of said CMV polypeptide consists of 5 to 15 consecutive amino acids. Preferably, said T helper cell epitope replaces a N-terminal region of said CMV polypeptide, and wherein said replaced N-terminal region of said CMV polypeptide consists of 5 to 15 consecutive amino acids, preferably of 9 to 14, 9 to 13 or 10 to 13 consecutive amino acids, more preferably of 11 to 13 consecutive amino acids, and most preferably of 11, 12 or 13 consecutive amino acids.

Antigenic CMV fusion polypeptide: The term “antigenic CMV fusion polypeptide”, as used herein refers to a polypeptide comprising, preferably consisting of, a CMV polypeptide, wherein said CMV polypeptide is modified to comprise an antigenic polypeptide as defined and described herein, and optionally further to comprise a T helper cell epitope. Typically and preferably, the antigenic CMV fusion polypeptide is capable of forming a modified virus-like particle of CMV upon expression by self-assembly. Thus, in a preferred embodiment, said antigenic CMV fusion polypeptide is capable of forming a modified virus-like particle of CMV by self-assembly, typically and preferably by self-assembly upon expression, typically and preferably upon expression in E. coli. In a further preferred embodiment, said antigenic CMV fusion polypeptide is co-expressed with a polypeptide selected from a modified CMV polypeptide, a CMV polypeptide and a CMV protein as defined herein, to form a mosaic fusion CMV VLP by self-assembly, typically and preferably by self-assembly upon expression, typically and preferably upon expression in E. coli. Typically and preferably, said T helper cell epitope replaces a N-terminal region of said CMV polypeptide, wherein said replaced N-terminal region of said CMV polypeptide consists of 5 to 15 consecutive amino acids. Preferably, said T helper cell epitope replaces a N-terminal region of said CMV polypeptide, and wherein said replaced N-terminal region of said CMV polypeptide consists of 5 to 15 consecutive amino acids, preferably of 9 to 14, 9 to 13 or 10 to 13 consecutive amino acids, more preferably of 11 to 13 consecutive amino acids, and most preferably of 11, 12 or 13 consecutive amino acids.

N-terminal region of the CMV polypeptide: The term “N-terminal region of the CMV polypeptide” as used herein, refers either to the N-terminus of said CMV polypeptide, and in particular to the N-terminus of a coat protein of CMV, or to the region of the N-terminus of said CMV polypeptide or said coat protein of CMV but starting with the second amino acid of the N-terminus of said CMV polypeptide or said coat protein of CMV if said CMV polypeptide or said coat protein comprises a N-terminal methionine residue. Preferably, in case said CMV polypeptide or said coat protein comprises a N-terminal methionine residue, from a practical point of view, the start-codon encoding methionine will usually be deleted and added to the N-terminus of the T helper (Th) cell epitope. Further preferably, one, two or three additional amino acids, preferably one amino acid, may be optionally inserted between the stating methionine and the T helper cell epitope for cloning purposes.

Recombinant polypeptide: In the context of the invention the term “recombinant” when used in the context of a polypeptide refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant modified virus-like particle: In the context of the invention the term “recombinant modified virus-like particle” refers to a modified virus-like particle (VLP) which is obtained by a process which comprises at least one step of recombinant DNA technology.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CMV. Thus, a mutated amino acid sequence differs from an amino acid sequence of a coat protein of CMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, 92%, 93% 94%, 95%, 96%, 97%, 98%, or 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion and amino acid exchange. Preferably, the mutated amino acid sequence differs from an amino acid sequence of a coat protein of CMV in least one amino acid, wherein preferably said difference is an amino acid exchange.

The terms “corresponding, correspond or corresponds” when used herein to describe the relationship of specific positions of amino acid residue(s) within polypeptides and amino acid sequences, respectively, refers to the position of an amino acid residue(s) within an amino acid sequence, which corresponds to given and specific amino acid residue(s) of another amino acid sequence that can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term “amino acid exchange” refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence.

The term “isoelectric point” as used herein and abbreviated as pI, refers to the pH at which a molecule carries no net electrical charge or is electrically neutral in the statistical mean. In particular, the term “isoelectric point” is used herein to refer to the pH at which antigens, used in the present invention and which are composed of amino acids, carries no net electrical charge or is electrically neutral in the statistical mean. At a pH below their pI, such antigens carry a net positive charge; above their pI they carry a net negative charge. Typically and preferably when referring to pI values, and in particular to pI values of antigens of the present invention and within the present disclosure, said pI values are determined by entering the primary amino acid sequence for a particular protein and antigen, respectively, into the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). Thus, if referred herein to the ExPASy Compute pI/MW tool is refers to the one described by Gasteiger et al. The tool calculates the theoretical isoelectric point pI and Mw of a specified Swiss-Prot/TrEMBL entry or a user-entered amino acid sequence. The pI of the protein is calculated using pk values of amino acids described in Bjellqvist et al., which were defined by examining polypeptide migration between pH 4.5 to 7.3 in an immobilised pH gradient gel environment with 9.2M and 9.8M urea at 15° C. or 25° C. (Bjellqvist, B. et al, 1993, Electrophoresis 14:1023-1031; Bjellqvist, B. er al, 1994, Electrophoresis 15:529-539).

Epitope: The term “epitope” refers to continuous or discontinuous portions of an a polypeptide or an antigen, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The terms “T helper cell epitope or Th cell epitope, as interchangeably used” and as used herein, refer to an epitope that is capable of recognition by a helper Th cell. Typically and preferably, the term “Th cell epitope” as used herein refers to a Th cell epitope that is capable of binding to at least one, preferably more than one MHC class II molecules. The simplest way to determine whether a peptide sequence is a Th cell epitope is to measure the ability of the peptide to bind to individual MHC class II molecules. This may be measured by the ability of the peptide to compete with the binding of a known Th cell epitope peptide to the MHC class II molecule. A representative selection of HLA-DR molecules are described in e.g. Alexander J, et al., Immunity (1994) 1:751-761. Affinities of Th cell epitopes for MHC class II molecules should be at least 10⁻⁵M. A representative collection of MHC class II molecules present in different individuals is given in Panina-Bordignon P, et al., Eur J Immunol (1989) 19:2237-2242. As a consequence, the term “Th cell epitope” as used herein preferably refers to a Th cell epitope that generates a measurable T cell response upon immunization and boosting. Moreover, and again further preferred, the term “Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Amino acid linker: The term “amino acid linker” as used herein, refers to a linker consisting exclusively of amino acid residues. The amino acid residues of the amino acid linker are composed of naturally occurring amino acids or unnatural amino acids known in the art, all-L or all-D or mixtures thereof. The amino acid residues of the amino acid linker are preferably naturally occurring amino acids, all-L or all-D or mixtures thereof. In a preferred embodiment, said amino acid linker consists of naturally occurring alpha amino acids, all in its L-configuration.

G-linker: The term “G-linker”, as used herein refers to an amino acid linker solely consisting of glycine amino acid residues. The G-linker in accordance with the present invention comprise at least two glycine residues and at most ten glycine residues.

GS-linker: The term “GS-linker”, as used herein refers to an amino acid linker solely consisting of glycine and serine amino acid residues. The GS-linker in accordance with the present invention comprise at least one glycine and at least one serine residue. Typically and preferably, the GS-linker has a length of at most 30 amino acids.

GS*-linker: The term “GS*-linker”, as used herein refers to an amino acid linker comprising at least one glycine, at least one serine and at least one amino acid residue selected from Thr, Ala, Lys, and Cys. Typically and preferably, the GS*-linker has a length of at most 30 amino acids.

The term “amino acid”, as used herein, refers to organic compounds containing the functional groups amine (—NH₂) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized; alpha-(α-), beta-(β-), gamma-(γ-) and delta-(δ-) etc. amino acids as well as mixtures thereof in any ratio; and, if applicable such as for alpha-(α-) amino acids, any isomeric form of an amino acid, i.e. its D-stereoisomers and L-stereoisomers (alternatively addressed by the (R) and(S) nomenclature) as well as mixtures thereof in any ratio including in a racemic ratio of 1:1. The term “D-stereoisomer”, “L-stereoisomer”, “D-amino acid” or “L-amino acid” refers to the chiral alpha carbon of the amino acids. In a preferred embodiment, the term amino acid refers to an alpha amino acid, preferably to a naturally occurring alpha amino acid, further preferably to a naturally occurring alpha amino acid in its L-configuration.

Associated: The terms “associated” or “association” as used herein refer to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds.

Attachment Site, First: As used herein, the phrase “first attachment site” refers to an element which is naturally occurring with the virus-like particle or which is artificially added to the virus-like particle, and to which the second attachment site may be linked. The first attachment site preferably is a protein, a polypeptide, an amino acid, a peptide, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid residue, preferably the amino group of the side chain of a lysine residue. In a further preferred embodiment, said first attachment site is an amino acid residue naturally occurring in said VLP. In another preferred embodiment, said first attachment site is an amino acid residue not naturally occurring in said VLP. The first attachment site is typically located on the surface, and preferably on the outer surface of the VLP. Multiple first attachment sites are present on the surface, preferably on the outer surface of the VLP, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP. In a very preferred embodiment said first attachment site is the amino group of a lysine residue of the amino acid sequence of said VLP polypeptide. In a further very preferred embodiment said first attachment site is the amino group of a lysine residue of the amino acid sequence of said VLP polypeptide, wherein said lysine residue is naturally occurring in said VLP polypeptide. In another very preferred embodiment said first attachment site is the amino group of a lysine residue of the amino acid sequence of said VLP polypeptide, wherein said lysine residue is not naturally occurring in said VLP polypeptide but artificially added, be it by, for example, by addition to said VLP polypeptide or by substitution of an amino acid naturally occurring in said VLP polypeptide. In a further very preferred embodiment said first attachment site is an amino acid residue naturally occurring in said VLP polypeptide. In a further very preferred embodiment said first attachment site is an amino acid residue not naturally occurring in said VLP polypeptide but artificially added, be it by, for example, by addition to said VLP polypeptide or by substitution of an amino acid naturally occurring in said VLP polypeptide.

Attachment Site, Second: As used herein, the phrase “second attachment site” refers to an element which is naturally occurring with or which is artificially added to the antigen and to which the first attachment site may be linked. The second attachment site of the antigen preferably is a protein, a polypeptide, a peptide, an amino acid, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group, preferably the sulfhydryl group of a cysteine residue. In a further preferred embodiment, said second attachment site is an amino acid residue naturally occurring in said antigen. In another preferred embodiment, said second attachment site is an amino acid residue not naturally occurring in said antigen. The term “antigen with at least one second attachment site” refers, therefore, to a construct comprising the antigen and at least one second attachment site. However, in particular for a second attachment site, which is not naturally occurring within the antigen, such a construct typically and preferably further comprises a “linker”. In another preferred embodiment the second attachment site is associated with the antigen through at least one covalent bond, preferably through at least one peptide bond. In a further embodiment, the second attachment site is naturally occurring within the antigen. In another further preferred embodiment, the second attachment site is artificially added to the antigen through a linker, wherein said linker comprises or alternatively consists of a cysteine. Preferably, the linker is fused to the antigen by a peptide bond. In a very preferred embodiment, the second attachment site is a sulfhydryl group, preferably the sulfhydryl group of a cysteine residue. Preferably, the linker is fused to the antigen by a peptide bond. In a further very preferred embodiment said second attachment site is an amino acid residue naturally occurring in said antigen. In a further very preferred embodiment said second attachment site is an amino acid residue not naturally occurring in said antigen but artificially added, be it by, for example, by addition to said antigen or by substitution of an amino acid naturally occurring in antigen.

Linked: The terms “linked” or “linkage” as used herein, refer to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide covalent bond(s). In other preferred embodiments the first attachment site and the second attachment site are linked through at least one covalent bond, preferably through at least one peptide bond, and even more preferably through exclusively peptide covalent bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker. In other preferred embodiments the first attachment site and the second attachment site are linked through at least one covalent bond, preferably through at least one peptide bond, and even more preferably through exclusively peptide bond(s). In a preferred embodiment, the first attachment site and the second attachment site are linked through one peptide bond representing a direct peptide linkage of the first attachment site and the second attachment site. In another preferred embodiment, the first attachment site and the second attachment site are linked through exclusively peptide bond(s) representing a peptide linkage of the first attachment site and the second attachment site by way of an amino acid linker as described herein. In case, the antigen, preferably the antigenic polypeptide is inserted within a VLP polypeptide by way of fusion, either two direct peptide linkages of first attachment sites and second attachment sites occur or one or two peptide linkages of first attachment sites and the second attachment sites occur by way of one or two identical or different amino acid linkers as described herein.

Linker: A “linker”, as used herein, either associates the second attachment site with the antigen or already comprises or consists of the second attachment site. Preferably, a “linker”, as used herein, already comprises the second attachment site, typically and preferably as one amino acid residue, preferably as a cysteine residue. A preferred linker is a linker containing at least one amino acid residue, or even more preferred is a linker consisting exclusively of amino acid residues. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Further linkers useful for the present invention are molecules comprising a C1-6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl-(C5, C6), aryl- or heteroaryl-moiety and additional amino acid(s) can also be used as linkers for the present invention and shall be encompassed within the scope of the invention. Association of the linker with the antigen is preferably by way of at least one covalent bond, more preferably by way of at least one peptide bond.

Antigen: The antigen of the present invention is a nerve growth factor (NGF) antigen. Thus, as used herein, the term “antigen” refers to a nerve growth factor (NGF) antigen that is typically and preferably capable of being bound by an antibody or a T-cell receptor (TCR) if presented by MHC molecules.

Antigenic polypeptide: As used herein, the term “antigenic polypeptide” refers to the nerve growth factor (NGF) antigen as defined herein, and which is a polypeptide comprised in the antigenic CMV fusion polypeptide as defined herein. The terms “antigenic polypeptide” and “antigenic NGF polypeptide” are interchangeably used herein.

Ordered and repetitive antigen array: As used herein, the term “ordered and repetitive antigen array” refers to a repeating pattern of antigen which typically and preferably is characterized by a high order of uniformity in spacial arrangement of the antigens with respect to the modified VLP of CMV. In one embodiment of the invention, the repeating pattern may be a geometric pattern. Certain embodiments of the invention, such as antigens linked to the modified VLP of CMV, are typical and preferred examples of suitable ordered and repetitive antigen arrays which, moreover, possess strictly repetitive paracrystalline orders of antigens, preferably with spacing of 1 to 30 nanometers, preferably 2 to 15 nanometers, even more preferably 2 to 10 nanometers, even again more preferably 2 to 8 nanometers, and further more preferably 1.6 to 7 nanometers.

Coupling efficiency: The coupling efficiency of a virus-like particle with a specific antigen is determined by SDS-PAGE of the coupling reactions. The intensities of Coomassie Blue-stained bands corresponding to components of the coupling reaction are determined by densitometry and used to calculate coupling efficiency. Coupling efficiency is defined as the ratio of (i) the amount of VLP polypeptides coupled to said antigen to (ii) the total amount of VLP polypeptides. Typically and preferably, said coupling efficiency is at least 5%, 10%, preferably at least 15%, further preferably at least 20%, 25% or at least 30%, and again further preferably of at least 35% or at least 40%. Coupling deficiency can also be expressed by the total number of antigens linked to the modified CMV VLP. Coupling deficiency can be dependent on the nature of the antigen, and the total numbers of antigens linked to the modified CMV VLP are typically and preferably at least 5, at least 7, at least 10, at least 15, at least 20, at least 25, at least 30, at least 40 and at least 50 antigens.

Nerve growth factor (NGF) antigen: The term “nerve growth factor (NGF) antigen” as used herein refers to an NGF antigen that comprises biological activity. A biological active NGF antigen as used herein generally refers to the ability to bind to the NGF high affinity receptor TrkA and/or activate NGF receptor signaling pathway. It typically and preferably is capable of inducing anti-NGF antibodies in canine, when administered to said canine in form of any of the inventive compositions, wherein said anti-NGF antibodies are capable of neutralizing the biological activity of nerve growth factor (NGF) in an in vitro assay, preferably as described herein (cf. Example 6). The term “biological activity” as used herein and when referring to the NGF antigen, refers to the activity of an NGF antigen in a cell proliferation assay, wherein preferably said cell proliferation assay is based on an NGF dependent human erythroleukemic TF-1 cell line, wherein still further preferably said cell proliferation assay is performed under conditions essentially as described in Example 6 herein. Moreover, NGF antigen typically refers to a polypeptide comprising, preferably consisting of, the amino acid sequence of canine or feline nerve growth factor or the corresponding orthologs from any other species, preferably from a non-human animal that shows biological activity. In particular it refers to a polypeptide comprising, preferably consisting of, the amino acid sequence of canine or feline nerve growth factor or the corresponding orthologs from any other species, preferably from a non-human animal, or to a polypeptide having a sequence identity of at least 90%, preferably of at least 92%, further preferably of at least 95%, and again further preferably of at least 98% with the amino acid sequence of canine or feline nerve growth factor or the corresponding orthologs from any other species, preferably from a non-human animal. Preferred NGF antigens from various animal species are canine NGF (cNGF), feline NGF (fNGF), equine NGF (eNGF), bovine NGF (bNGF) and porcine NGF (pNGF), preferably canine NGF (cNGF) or feline NGF (fNGF), and said NGF antigens comprise, preferably consists of, the polypeptides of SEQ ID NO: 30, SEQ ID NO:31, SEQ ID NO:33, SEQ ID NO:54, SEQ ID NO:55, SEQ ID NO: 56, SEQ ID NO:57, and SEQ ID NO:58, or an amino acid sequence having a sequence identity of at least 90%, preferably of at least 92%, further preferably of at least 95%, and again further preferably of at least 98% with any of SEQ ID NO:30, SEQ ID NO:31, SEQ ID NO: 33, SEQ ID NO:54, SEQ ID NO:55, SEQ ID NO:56, SEQ ID NO:57, and SEQ ID NO: 58.

Endogenous or self NGF protein: The term “endogenous or self NGF protein” as used herein refers to the NGF protein of the animal that receive the treatment according to this invention. In this context, terms “self NGF” and “endogenous NGF” are interchangeable used.

NGF-containing CMV VLP composition(s): The term “NGF-containing CMV VLP composition(s)” as used herein refers to a composition, comprising (a) CVM VLP comprising at least one attachment site and (b) at least one NGF antigen comprising at least a second attachment site, and wherein (a) and (b) are linked through said at least one first and said at least one second attachment site.

Adjuvant: The term “adjuvant” as used herein refers to stimulators of the immune response and/or substances that typically allow generation of a depot in the host which when combined with the composition, vaccine or pharmaceutical composition, respectively, of the present invention may provide for an more enhanced immune response. Adjuvants of varying types with different mechanisms of action are described and are able to enhance the antigen-specific antibody response (Pulendran B et al, 2021, Nature Reviews Drug Discovery 20:454-475). Typical and preferred adjuvants are mineral salts (e.g. Aluminum Hydroxide, Aluminum Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). The term “adjuvant” as used herein may also comprise mixtures of adjuvants. Virus-like particles have sometimes been described as an adjuvant. However, the term “adjuvant”, as used within the context of this application, refers to an adjuvant not being the inventive modified virus-like particle. Rather “adjuvant” relates to an additional, distinct component of the inventive compositions, vaccines or pharmaceutical compositions.

Immunostimulatory substance: As used herein, the term “immunostimulatory substance” refers to a substance capable of inducing and/or enhancing an immune response. Immunostimulatory substances, as used herein, include, but are not limited to, toll-like receptor activating substances and substances inducing cytokine secretion. Toll-like receptor activating substances include, but are not limited to, immunostimulatory nucleic acids, peptideoglycans, lipopolysaccharides, lipoteichonic acids, imidazoquinoline compounds, flagellins, lipoproteins, and immunostimulatory organic substances such as taxol.

Immunostimulatory nucleic acid (ISS-NA): As used herein, the term “immunostimulatory nucleic acid” refers to a nucleic acid capable of inducing and/or enhancing an immune response. Immunostimulatory nucleic acids comprise ribonucleic acids and in particular deoxyribonucleic acids, wherein both, ribonucleic acids and deoxyribonucleic acids may be either double stranded or single stranded. Preferred ISS-NA are deoxyribonucleic acids, wherein further preferably said deoxyribonucleic acids are single stranded. Preferably, immunostimulatory nucleic acids contain at least one CpG motif comprising an unmethylated C. Very preferred immunostimulatory nucleic acids comprise at least one CpG motif, wherein said at least one CpG motif comprises or preferably consist of at least one, preferably one, CG dinucleotide, wherein the C is unmethylated. Preferably, but not necessarily, said CG dinucleotide is part of a palindromic sequence. The term immunostimulatory nucleic acid also refers to nucleic acids that contain modified bases, preferably 4-bromo-cytosine. Specifically preferred in the context of the invention are ISS-NA which are capable of stimulating IFN-alpha production in dendritic cells. Immunostimulatory nucleic acids useful for the purpose of the invention are described, for example, in WO2007/068747A1.

Oligonucleotide: As used herein, the term “oligonucleotide” refers to a nucleic acid sequence comprising two or more nucleotides, preferably about 6 to about 200 nucleotides, and more preferably 20 to about 100 nucleotides, and most preferably 20 to 40 nucleotides. Oligonucleotides are polyribonucleotides or polydeoxribonucleotides and are preferably selected from (a) unmodified RNA or DNA, and (b) modified RNA or DNA. The modification may comprise the backbone or nucleotide analogues. Oligonucleotides are preferably selected from the group consisting of (a) single- and double-stranded DNA, (b) DNA that is a mixture of single- and double-stranded regions, (c) single- and double-stranded RNA, (d) RNA that is mixture of single- and double-stranded regions, and (e) hybrid molecules comprising DNA and RNA that are single-stranded or, more preferably, double-stranded or a mixture of single- and double-stranded regions. Preferred nucleotide modifications/analogs are selected from the group consisting of (a) peptide nucleic acid, (b) inosin, (c) tritylated bases, (d) phosphorothioates, (e) alkylphosphorothioates, (f) 5-nitroindole desoxyribofliranosyl, (g) 5-methyldesoxycytosine, and (h) 5,6-dihydro-5,6-dihydroxydesoxythymidine. Phosphorothioated nucleotides are protected against degradation in a cell or an organism and are therefore preferred nucleotide modifications. Unmodified oligonucleotides consisting exclusively of phosphodiester bound nucleotides, typically are more active than modified nucleotides and are therefore generally preferred in the context of the invention. Most preferred are oligonucleotides consisting exclusively of phosphodiester bound oligonucleotides, wherein further preferably said oligonucleotides are single stranded. Further preferred are oligonucleotides capable of stimulating IFN-alpha production in cells, preferably in dendritic cells. Very preferred oligonucleotides capable of stimulating IFN-alpha production in cells are selected from A-type CpGs and C-type CpGs. Further preferred are RNA-molecules without a Cap.

CpG motif: As used herein, the term “CpG motif” refers to a pattern of nucleotides that includes an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5′ and 3′ ends. Without being bound by theory, the bases flanking the CpG confer a significant part of the activity to the CpG oligonucleotide.

Unmethylated CpG-containing oligonucleotide: As used herein, the term “unmethylated CpG-containing oligonucleotide” or “CpG” refers to an oligonucleotide, preferably to an oligodeoxynucleotide, containing at least one CpG motif. Thus, a CpG contains at least one unmethylated cytosine, guanine dinucleotide. Preferred CpGs stimulate/activate, e.g. have a mitogenic effect on, or induce or increase cytokine expression by, a vertebrate bone marrow derived cell. For example, CpGs can be useful in activating B cells, NK cells and antigen-presenting cells, such as dendritic cells, monocytes and macrophages. Preferably, CpG relates to an oligodeoxynucleotide, preferably to a single stranded oligodeoxynucleotide, containing an unmethylated cytosine followed 3′ by a guanosine, wherein said unmethylated cytosine and said guanosine are linked by a phosphate bond, wherein preferably said phosphate bound is a phosphodiester bound or a phosphorothioate bound, and wherein further preferably said phosphate bond is a phosphodiester bound. CpGs can include nucleotide analogs such as analogs containing phosphorothioester bonds and can be double-stranded or single-stranded. Generally, double-stranded molecules are more stable in vivo, while single-stranded molecules have increased immune activity. Preferably, as used herein, a CpG is an oligonucleotide that is at least about ten nucleotides in length and comprises at least one CpG motif, wherein further preferably said CpG is 10 to 60, more preferably 15 to 50, still more preferably 20 to 40, still more preferably about 30, and most preferably exactly 30 nucleotides in length. A CpG may consist of methylated and/or unmethylated nucleotides, wherein said at least one CpG motif comprises at least one CG dinucleotide wherein the C is unmethylated. The CpG may also comprise methylated and unmethylated sequence stretches, wherein said at least one CpG motif comprises at least one CG dinucleotide wherein the C is unmethylated. Very preferably, CpG relates to a single stranded oligodeoxynucleotide containing an unmethylated cytosine followed 3′ by a guanosine, wherein said unmethylated cytosine and said guanosine are linked by a phosphodiester bound. The CpGs can include nucleotide analogs such as analogs containing phosphorothioester bonds and can be double-stranded or single-stranded. Generally, phosphodiester CpGs are A-type CpGs as indicated below, while phosphothioester stabilized CpGs are B-type CpGs. Preferred CpG oligonucleotides in the context of the invention are A-type CpGs.

A-type CpG: As used herein, the term “A-type CpG” or “D-type CpG” refers to an oligodeoxynucleotide (ODN) comprising at least one CpG motif. A-type CpGs preferentially stimulate activation of T cells and the maturation of dendritic cells and are capable of stimulating IFN-alpha production. In A-type CpGs, the nucleotides of the at least one CpG motif are linked by at least one phosphodiester bond. A-type CpGs comprise at least one phosphodiester bond CpG motif which may be flanked at its 5′ end and/or, preferably and, at its 3′ end by phosphorothioate bound nucleotides. Preferably, the CpG motif, and hereby preferably the CG dinucleotide and its immediate flanking regions comprising at least one, preferably two nucleotides, are composed of phosphodiester nucleotides. Preferred A-type CpGs exclusively consist of phosphodiester (PO) bond nucleotides. Typically and preferably, the poly G motif comprises or alternatively consists of at least one, preferably at least three, at least 4, 5, 6, 7, 8, 9, 10, 11, 12, 13, 14, or 15 G's (guanosines), most preferably by at least 10 G's. Preferably, the A-type CpG of the invention comprises or alternatively consists of a palindromic sequence.

Packaged: The term “packaged” as used herein refers to the state of a polyanionic macromolecule or immunostimulatory substances in relation to the core particle and VLP, respectively. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule or immunostimulatory substances can be enclosed by the VLP without the existence of an actual binding, in particular of a covalent binding. In preferred embodiments, the at least one polyanionic macromolecule or immunostimulatory substances is packaged inside the VLP, most preferably in a non-covalent manner. In case said immunostimulatory substances is nucleic acid, preferably a DNA, the term packaged implies that said nucleic acid is not accessible to nucleases hydrolysis, preferably not accessible to DNAse hydrolysis (e.g. DNaseI or Benzonase), wherein preferably said accessibility is assayed as described in Examples 11-17 of WO2003/024481A2.

Effective amount: As used herein, the term “effective amount” refers to an amount necessary or sufficient to realize a desired biologic effect. An effective amount of the composition, or alternatively the pharmaceutical composition, would be the amount that achieves this selected result, and such an amount could be determined as a matter of routine by a person skilled in the art. The effective amount can vary depending on the particular composition being administered and the size of the subject. One of ordinary skill in the art can empirically determine the effective amount of a particular composition of the present invention without necessitating undue experimentation. Preferably, the term “effective amount” refers to an amount that (i) treats or prevents the particular disease or disorder, (ii) attenuates, ameliorates, or eliminates one or more symptoms of the particular disease or disorder, or (iii) prevents or delays the onset of one or more symptoms of the particular disease or disorder, described herein.

Canine: The term “canine” as used herein preferably refers to a domestic dog (dog).

Veterinary composition: As used herein, the term “veterinary composition” refers to a composition suitable for use in non-human animals.

NGF-related disorder: The term “NGF-related disorder” as used herein refers to any disorder that is caused by any dysfunction or dysregulation of the NGF/TrkrA receptor pathway. NGF related disorder, include but are not limited to, pain such for example nociceptive pain, inflammatory pain, postsurgical pain, pain associated with musculoskeletal disorders such as rheumatoid arthritis, osteoarthritis, ankylosing spondylitis, seronegative (non-rheumatoid) arthropathies, non-articular rheumatism and periarticular disorders, pain associated with degenerative joint disease, such as for example arthritis or osteoarthritis. Such pain can be of acute, chronic and/or refractory nature.

Pain: The term “Pain” as used herein, refers to pain of any etiology, including acute and chronic pain, and any pain with an inflammatory component. Examples of pain include nociceptive pain, inflammatory pain, postsurgical pain, pain associated with musculoskeletal disorders such as rheumatoid arthritis, osteoarthritis, ankylosing spondylitis, seronegative (non-rheumatoid) arthropathies, non-articular rheumatism and periarticular disorders, pain associated with degenerative joint disease, such as for example arthritis or osteoarthritis associated pain.

Acute pain: The term “acute pain” as used herein refers to sudden or urgent pain. Acute pain, for instance, can be caused by surgery or acute inflammation.

Chronic pain: The term “chronic pain” as used herein, refers to pain that is ongoing and usually lasts longer than six months. Chronic pain is not simply a temporal continuum of acute pain. In the setting of persistent injury, functional and structural reorganization of neuronal circuits in the CNS leads to long-term changes in perception and behavior. Such pain can persist after an injury or illness with pain signals remaining active in the nervous system for weeks, months or years.

Refractory pain: The term “refractory pain” as used herein, refers to pain that cannot be alleviated with conventional painkillers including anti-inflammatory compounds such as NSAIDs, corticosteroids and opioid analgesics.

Nociceptive pain: The term “The term “nociceptive pain” as used herein, refers to a pain arising from the stimulation of the pain receptors, due to injury, surgery or disease that affect the tissues, such as arthritis. Nociceptive pain also includes chronic pain. Preferred types of nociceptive pain are osteoarthritic associated pain, rheumatoid arthritis pain.

Inflammatory or inflammatory-related pain: The term “inflammatory or inflammatory-related pain” as used herein refers to the spontaneous hypersensitivity to pain that occurs in response to tissue damage and inflammation (e.g., postoperative pain, trauma, arthritis). Inflammatory pain is a type of nociceptive pain that results from activation and sensitization of nociceptors by inflammatory mediators. Often the pain improves when the inflammation subsides.

Postsurgical pain: The term “postsurgical pain” as used herein refers to pain arising or resulting from an external trauma such as a cut, puncture, incision, tear, or wound into tissue of an individual (including that that arises from all surgical procedures, whether invasive or non-invasive). As used herein, post-surgical pain does not include pain that occurs (arises or originates) without an external physical trauma.

Pain associated with musculoskeletal diseases: The term “pain associated with musculoskeletal diseases” as used herein refers to pain associated with musculoskeletal disorders such as rheumatoid arthritis, osteoarthritis, ankylosing spondylitis, seronegative (non-rheumatoid) arthropathies, non-articular rheumatism.

Pain associated with degenerative joint disease: As used herein the term “pain associated with degenerative joint disease” refers to pain associated with arthritis or osteoarthritis associated pain.

Osteoarthritis (OA)-associated pain: The term “Osteoarthritis (OA)-associated pain or OA-associated pain” as used herein refers to a chronic joint disease characterized by loss of joint cartilage, thickening of the joint capsule and new bone formation around the joint (osteophytosis) and ultimately leading to pain and limb dysfunction. In canines, signs of OA are often non-specific and include: i) activity impairment, reluctance to exercise, decrease in overall activity, stiffness, lameness, inability to jump, changes in gait such as “bunny-hopping”, ii) pain on manipulation, behavioral changes such as aggression or signs of discomfort.

Chronic refractory OA-associated pain: The term “chronic refractory OA-associated pain” as used herein refers to a chronic pain that does not respond or only slightly responds to conventional treatment including NSAIDs, corticoids and opioids.

Treatment: As used herein, the terms “treatment”, “treat”, “treated” or “treating” refer a therapy. Treatment refers to an approach for obtaining beneficial or desired clinical results. For purposes of this invention, beneficial or desired clinical results include, but are not limited to, one or more of the following: improvement or alleviation of any aspect of NGF-related disorder, in particular pain, selected from the group consisting of acute, chronic, refractory pain, nociceptive pain, inflammatory pain, postsurgical pain, pain associated with musculoskeletal disorders such as rheumatoid arthritis, osteoarthritis, ankylosing spondylitis, seronegative (non-rheumatoid) arthropathies, non-articular rheumatism and periarticular disorders, pain associated with degenerative joint disease, such as for example arthritis or osteoarthritis associated pain. For purposes of this invention, beneficial or desired clinical results include, but are not limited to, one or more of the following: including lessening severity, incidence, alleviation of one or more symptoms associated with the NGF-related disorder, preferably pain including any aspect of pain (such as shortening duration of the NGF-related disorder, preferably pain, reduction of the NGF related disorder, preferably pain sensitivity or pain sensation). For instance, one parameter to measure the lessening severity, incidence or alleviation of pain associated with OA is the reduction of lameness in treated animals as compared to non-treated animals.

### Method of Treatment

The present invention provides for a composition for use in a method of treating a NGF-related disorder in canine comprising the administration of said composition to said canine, where the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

According to further embodiment, the composition for use in a method of treating a NGF-related disorder in canine is an immunogenic composition, and wherein such treatment comprises, preferably consists of the active immunization against endogenous NGF protein of said canine.

Thus, according to a further aspect, the present invention provides for a composition for use in a method of treating a NGF-related disorder in canine comprising the administration of said composition to said canine, where the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the treatment consists in an active immunization of said canine against endogenous NGF-protein of said canine.

According to a further aspect the present invention provides a method for the treatment of NGF-related disorder in canine, comprising the administration of a composition to said canine, wherein said composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

According to a further aspect the present invention provides a method for the active immunization of canine against an NGF-related disorder, comprising the administration of a composition to said canine, wherein said composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In a preferred embodiment, the NGF-related disorder is pain, preferably pain selected from the group of nociceptive pain, inflammatory pain, postsurgical pain, pain associated with musculoskeletal diseases, pain associated with degenerative joint disease, and/or osteoarthritis (OA)-associated pain.

Thus, according to a further aspect, the present invention provides for a composition for use in a method of treating NGF-related disorder in canine comprising the administration of such composition to said canine, where the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is pain.

According to a further aspect, the present invention provides for a composition for use in a method of treating NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is pain selected from the group consisting of nociceptive pain, inflammatory pain, postsurgical pain, pain associated with musculoskeletal diseases, pain associated with degenerative joint disease and/or osteoarthritis (OA)-associated pain, preferably wherein such pain is OA-associated pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is nociceptive pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is inflammatory pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is postsurgical pain.

According to a further aspect, the present invention provides for a composition for use in a method of treating NGF-related disorder in a canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is pain associated with musculoskeletal diseases.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is pain associated with degenerative joint disease.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is osteoarthritis (OA)-associated pain.

The respective NGF-related disorder, preferably pain, more preferably the pain selected from the group consisting of nociceptive pain, inflammatory pain, postsurgical pain, pain associated with musculoskeletal diseases, pain associated with degenerative joint disease, and/or osteoarthritis (OA)-associated pain, preferably such OA-associated pain, is of acute, chronic or refractory origin.

According to a further aspect, the present invention provides for a composition for use in a method of treating NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is an acute NGF-related disorder.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is acute pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is acute pain selected from the group consisting of acute nociceptive pain, acute inflammatory pain, acute postsurgical pain, acute pain associated with musculoskeletal diseases, acute pain associated with degenerative joint disease, and/or acute osteoarthritis (OA)-associated pain, preferably wherein such pain is acute pain associated with degenerative joint disease, even more preferred acute OA-associated pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is a chronic NGF-related disorder.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is chronic pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is chronic pain selected from the group consisting of chronic nociceptive pain, chronic inflammatory pain, chronic postsurgical pain, chronic pain associated with musculoskeletal diseases, chronic pain associated with degenerative joint disease, and/or chronic osteoarthritis (OA)-associated pain, preferably wherein such chronic pain is chronic pain associated with degenerative joint disease, even more preferred chronic OA-associated pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is a refractory NGF-related disorder.

According to a further aspect, the present invention provides for a composition for use in a method of treating NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is refractory pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is refractory pain selected from the group consisting of refractory nociceptive pain, refractory inflammatory pain, refractory postsurgical pain, refractory pain associated with musculoskeletal diseases, refractory pain associated with degenerative joint disease and/or refractory osteoarthritis (OA)-associated pain, preferably wherein such refractory pain is refractory pain associated with degenerative joint disease, even more preferred refractory OA-associated pain.

According to a further aspect, the present invention provides for a composition for use in a method of treating NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is a chronic refractory NGF-related disorder.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is chronic refractory pain.

According to a further aspect, the present invention provides for a composition for use in a method of treating NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site; and wherein the NGF-related disorder is chronic refractory pain selected from the group consisting of chronic refractory nociceptive pain, chronic refractory inflammatory pain, chronic refractory postsurgical pain, chronic refractory pain associated with musculoskeletal diseases and/or chronic refractory osteoarthritis (OA)-associated pain, preferably wherein such chronic refractory pain is chronic refractory pain associated with degenerative joint disease, even more preferred chronic refractory OA-associated pain.

### Dosing:

The compositions used according to the invention are normally administered in an effective dose to treat the NGF-related disorder, preferably pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to preferred embodiment of the invention, the composition for use in a method of treating NGF-related disorder in canine, is administered to such canine in an amount of at least 50 μg/dose, preferably in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose.

According to a further aspect, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site and wherein said antigen is a nerve growth factor (NGF) antigen; and
wherein (a) and (b) are linked through said at least one first and said at least one second attachment site; and wherein the NGF-related disorder is pain, preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain; and wherein the composition is administered to such canine in an amount of at least 50 μg/dose, preferably in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose.

### Administration Regimen:

The NGF containing CMV VLP compositions were able to booster the neutralising anti-NGF antibodies after repeated or multiple dosing by not causing any long-term immune tolerance (i.e. not circumventing immune tolerance permanently), which would cause serious side effects and lessens the effect of the booster against endogenous NGF protein. This transiently breaking through the immune-tolerance without breaking the immune-tolerance completely against the endogenous NGF protein after repeated administration is key for the therapeutic benefit in managing NGF-related disorder, preferably in managing NGF-related pain. Moreover, a further requirement is that the vector (i.e. the CMV VLPs) as such presenting the NGF-antigen, does not cause a neutralizing immune response against such vector, as this would diminish the booster effect after any repeat dosing. It has been surprisingly found that the NGF-containing CMV VLPs are highly suitable for the management of NGF-related disorders in canine, in particular for the management of pain. The NGF-containing CMV VLPs surprisingly mediates a transient breaking through the immune-tolerance against endogenous NGF protein and activates and boosters an antibody-mediated inhibition of NGF binding to TrkA high affinity receptor and, thereby, prevents subsequent cascade activation without breaking permanently the immune-tolerance against endogenous NGF protein.

Thus, according to a further aspect the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in one or several doses.

In a further aspect, the NGF-containing CMV VLP composition is administered in at least two doses, preferably with a time interval between the first and the second dose of 7 to 21 days, preferably 14 to 21 days. Such preferred time interval between the first and the second dose can be 7, 8, 9, 10, 11, 12, 13, 14, 15, 16, 17, 18, 19, 20 or 21 days, preferably 14, 15, 16, 17, 18, 19, 20 or 21 days.

Thus, according to a further aspect the present invention provides for a composition for use in a method of treating a NGF-related disorder, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days.

In case of repeated dosing, the time intervals between the first and the second dose is preferably at least 7 days, more preferably at least 14 days, even more preferably between 7 and 21 days, even more preferably between 14 and 21 days. Further booster administrations are normally provided with longer time intervals to the previous administration. For example, if a third administration is provided, in general the time interval between the second and the third administration is between two to six months. For any further booster administration, the time interval following the previous booster administration (e.g. following the third administration) is preferably at least three months, preferably between three to six months.

Thus, according to a further aspect the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses.

According to a further aspect the present invention provides for a composition for use in a method of treating a NGF-related disorder, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses, and wherein the time intervals between the first and the second dose is preferably at least 7 days, preferably at least 14 days, more preferably between 7 and 21 days, more preferably between 14 and 21 days.

According to a further aspect the present invention provides for a composition for use in a method of treating a NGF-related disorder, in a canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses, and wherein the time intervals between the first and the second dose is preferably at least 7 days, preferably at least 14 days, more preferably between 7 and 21 days, more preferably between 14 and 21 days, and wherein the time interval for any subsequent administration is at least two to six months from the previous administration. Such time interval for any subsequent administration from the previous administration can be for example 2, 3, 4, 5, or 6 months, or any other time interval within the two to six months.

According to a further aspect the present invention provides for a composition for use in a method of treating a NGF-related disorder, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses, and wherein the time intervals between the first and the second dose is preferably two to three weeks (14 to 21 days), and wherein the time interval for any subsequent administration is about six months from the previous administration.

For the treatment of acute NGF related disorder, preferably of acute pain, in general one or two doses of the NGF containing CMV VLP composition are administered. If two doses are administered, normally a time interval of one to three weeks, preferably two to three weeks is chosen to obtain a maximum booster effect.

According to a further aspect the present invention provides for a composition for use in a method of treating an acute NGF-related disorder, preferably acute pain in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in at least two doses, preferably in two doses, preferably with a time interval between the first and the second dose of 7 to 21 days, preferably of 14 to 21 days (two to three weeks), more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. Such acute NGF-related disorder, preferably such acute pain, is selected from the group consisting of acute nociceptive pain, acute inflammatory pain, acute postsurgical pain, acute pain associated with musculoskeletal diseases, acute pain associated with degenerative joint disease, and/or acute osteoarthritis (OA)-associated pain, preferably such acute pain is acute pain associated with degenerative joint disease, more preferably acute OA-associated pain. Preferably, the NGF-containing CMV VLPs are administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose.

For the treatment of chronic or chronic refractory NGF-related disorder, preferably chronic pain or chronic refractory pain, repeated administrations can further enhance the therapeutic effect. If three to four doses are administered, normally the time interval between the first and second dose is 7 to 21 days, preferably 14 to 21 days (two to three weeks), more preferably 7, 8, 9, 10, 11, 12, 13, 14, 15, 16, 17, 18, 19, 20 or 21 days, more preferably 14, 15, 16, 17, 18, 19, 20 or 21 days followed by a time interval of two to six months for any subsequent administration to the previous administration. For example, the time interval between the second and third administration can be, two, three, four, five or six months (or any other interval in between), and for any fourth administration, three, four, five or six months (or any other interval in between) to the third administration. In general, time intervals of six months between the priming, which preferably consists of one or two doses, and any subsequent administration to the previous administration are highly desirable. Furthermore, the exact time interval for any further booster administration following the one or two priming administration(s) can be determined by a person skilled in the art depending on the anti-NGF-titers detected in the pre-treated animal (pre-treated canine).

According to a further aspect the present invention provides for a composition for use in a method of treating a chronic NGF-related disorder, preferably chronic pain, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses, wherein the repeated dosing comprises, or consists of the administration of one or two priming doses, preferably with two priming doses with a time interval between the first and second priming dose of 7 to 21 days, preferably of 14 to 21 days (two to three weeks), more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days; and wherein the repeated dosing comprises or consists of subsequent booster administrations, preferably with a time interval of two to six months, preferably three, four, five or six months, more preferably of four to six months to any previous administration. Such chronic NGF-related disorder, preferably such chronic pain, is selected from the group consisting of chronic nociceptive pain, chronic inflammatory pain, chronic postsurgical pain, chronic pain associated with musculoskeletal diseases, chronic pain associated with degenerative joint disease, and/or chronic osteoarthritis (OA)-associated pain, preferably such chronic pain is chronic pain associated with degenerative joint disease, more preferably chronic OA-associated pain. According to a further embodiment, such chronic NGF-related disorder can be chronic refractory pain selected from the group consisting of chronic refractory nociceptive pain, chronic refractory inflammatory pain, chronic refractory postsurgical pain, chronic refractory pain associated with musculoskeletal diseases and/or chronic refractory osteoarthritis (OA)-associated pain, preferably such chronic pain is chronic refractory pain associated with degenerative joint disease, more preferably chronic refractory OA-associated pain. Preferably, said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose.

According to a further aspect the present invention provides for a composition for use in a method of treating a chronic NGF-related disorder, preferably chronic pain, in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is administered to said canine in repeated doses, wherein the repeated dosing comprises, or consists of the administration two priming doses with a time interval between the first and second priming dose of two to three weeks, and the administration of a least two subsequent booster administrations, preferably with a time interval of two to three months between the 2nd priming administration and the first booster administration, and at least one further booster administration with a time interval of four to six months to any previous booster administration. Such chronic NGF-related disorder, preferably such chronic pain, is selected from the group consisting of chronic nociceptive pain, chronic inflammatory pain, chronic postsurgical pain, chronic pain associated with musculoskeletal diseases, chronic pain associated with degenerative joint disease, and/or chronic osteoarthritis (OA)-associated pain, preferably such chronic pain is chronic pain associated with degenerative joint disease, preferably chronic OA-associated pain. According to a further embodiment, such chronic NGF-related disorder can be chronic refractory pain selected from the group consisting of chronic refractory nociceptive pain, chronic refractory inflammatory pain, chronic refractory postsurgical pain, chronic refractory pain associated with musculoskeletal diseases and/or chronic refractory osteoarthritis (OA)-associated pain, preferably such chronic pain is chronic refractory pain associated with degenerative joint disease, preferably chronic refractory OA-associated pain.

Preferably said (NGF-containing CMV VLP) compositions care administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose.

### Ways of Administration:

In general, the NGF-containing CM VLP composition is systemically administered, preferably subcutaneously, intramuscularly or transdermal. In a preferred embodiment, the NGF-containing CM VLP composition is administered subcutaneously.

According to the further aspect, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein the composition is systemically administered, preferably subcutaneously, intramuscularly or transdermal. In a preferred embodiment, said NGF-containing CMV VLP composition is administered subcutaneously.

According to the further aspect, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, and wherein said composition is subcutaneously administered. Preferably, the NGF-related disorder is pain, preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. According to a further preferred embodiment, the systemically, preferably subcutaneously administered composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. According to a further preferred embodiment, the composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably between 14, 15, 16, 17, 18, 19, 20 and 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

### Ngf Antigen to be Used According to the Invention:

In another preferred embodiment, said antigen/NGF antigen is nerve growth factor (NGF) selected from human NGF (hNGF), canine NGF (cNGF), feline NGF (fNGF), equine NGF (eNGF), bovine NGF (bNGF) and porcine NGF (pNGF), preferably canine NGF (cNGF) or feline NGF (fNGF), and wherein further preferably said antigen is canine NGF (cNGF). In a preferred embodiment, said antigen comprises, or preferably consists of, of an amino acid sequence selected from any of SEQ ID NO:30, SEQ ID NO:31, SEQ ID NO:33, SEQ ID NO:54, SEQ ID NO:55, SEQ ID NO:56, SEQ ID NO:57, and SEQ ID NO:58, or an amino acid sequence having a sequence identity of at least 90% or at least 91%, preferably of at least 92%, at least 93% or at least 94%, further preferably of at least 95%, at least 96% or at least 97%, again further preferably of at least 98% or at least 99% with any of SEQ ID NO: 30, SEQ ID NO:31, SEQ ID NO:33, SEQ ID NO:54, SEQ ID NO:55, SEQ ID NO:56, SEQ ID NO:57, and SEQ ID NO:58. In a further embodiment, said NGF antigen comprises a polyhistidine-tag of at least two consecutive and at most 12 consecutive histidine residues, preferably C-terminally or N-terminally positioned of the NGF antigen. In a further preferred embodiment, said NGF antigen comprises a polyhistidine-tag of at least 2, 3, 4, 5, 6, 7, 8, 9, 10, 11 or 12 consecutive histidine residues, preferably C-terminally or N-terminally positioned of the NGF antigen. In a further embodiment, said NGF antigen comprises a polyhistidine-tag of 4, 6, 8 or 10 consecutive histidine residues, preferably C-terminally or N-terminally positioned of the NGF antigen. In a further embodiment, said NGF antigen comprises a polyhistidine-tag of 4 consecutive histidine residues, preferably C-terminally positioned of the NGF antigen. In a further embodiment, said NGF antigen comprises a polyhistidine-tag of 4 consecutive histidine residues, preferably N-terminally positioned of the NGF antigen. In a further embodiment, said NGF antigen comprises a polyhistidine-tag of 6 consecutive histidine residues consisting of SEQ ID NO:34, preferably C-terminally positioned of the NGF antigen. In a further embodiment, said NGF antigen comprises a polyhistidine-tag of 6 consecutive histidine residues consisting of SEQ ID NO:34, preferably N-terminally positioned of the NGF antigen. In a further embodiment, said NGF antigen comprises a polyhistidine-tag of 8 consecutive histidine residues, preferably C-terminally positioned of the NGF antigen. In a further embodiment, said NGF antigen comprises a polyhistidine-tag of 8 consecutive histidine residues, preferably N-terminally positioned of the NGF antigen. In a further embodiment, said NGF antigen comprises a polyhistidine-tag of 10 consecutive histidine residues, preferably C-terminally or N-terminally positioned of the NGF antigen.

In an alternative embodiment, said antigen is human NGF. In a further embodiment, said antigen comprises, or preferably consists of, SEQ ID NO:54 or an amino acid sequence having a sequence identity of at least 90%, preferably of at least 92%, further preferably of at least 95%, and again further preferably of at least 98% amino acid sequence identity with SEQ ID NO:54. In a further embodiment, said antigen comprises SEQ ID NO:54. In a further, said antigen consists of SEQ ID NO:54.

In a further very preferred embodiment, said antigen is canine NGF. In a very preferred embodiment, said antigen comprises, or preferably consists of, SEQ ID NO:30, SEQ ID NO: 31, SEQ ID NO:33 or an amino acid sequence having a sequence identity of at least 90%, preferably of at least 92%, further preferably of at least 95%, and again further preferably of at least 98% amino acid sequence identity with SEQ ID NO:30 or SEQ ID NO: 31 or SED ID NO: 33. In a further preferred embodiment, said antigen comprises SEQ ID NO: 30 or SEQ ID NO:31 or SEQ ID NO:33. In a further preferred embodiment, said antigen consists of SEQ ID NO:30 or SEQ ID NO:31 or SEQ ID NO:33. In a further very preferred embodiment, said antigen comprises, or preferably consists of, SEQ ID NO:30 or an amino acid sequence having a sequence identity of at least 90%, preferably of at least 92%, further preferably of at least 95%, and again further preferably of at least 98% amino acid sequence identity with SEQ ID NO:30. In a further very preferred embodiment, said antigen comprises SEQ ID NO:30. In a further very preferred embodiment, said antigen consists of SEQ ID NO:30. In a further preferred embodiment, said antigen comprises, or preferably consists of, SEQ ID NO:31 or an amino acid sequence having a sequence identity of at least 90%, preferably of at least 92%, further preferably of at least 95%, and again further preferably of at least 98% amino acid sequence identity with SEQ ID NO:31. In a further very preferred embodiment, said antigen comprises SEQ ID NO:31. In a further very preferred embodiment, said antigen consists of SEQ ID NO:31. In a further preferred embodiment, said antigen comprises, or preferably consists of, SEQ ID NO:33 or an amino acid sequence having a sequence identity of at least 90%, preferably of at least 92%, further preferably of at least 95%, and again further preferably of at least 98% amino acid sequence identity with SEQ ID NO:33. In a further very preferred embodiment, said antigen comprises SEQ ID NO:33. In a further very preferred embodiment, said antigen consists of SEQ ID NO: 33.

In a further embodiment, said antigen comprises, or preferably consists of, SEQ ID NO: 30 or an amino acid sequence having a sequence identity of at least 90%, preferably of at least 92%, further preferably of at least 95%, and again further preferably of at least 98% amino acid sequence identity with SEQ ID NO:30, and wherein said NGF antigen further comprises a polyhistidine-tag of at least two consecutive and at most 12 consecutive histidine residues, preferably 4, 6, 8, or 10 consecutive histidine residues, further preferably 6 consecutive histidine residues consisting of SEQ ID NO:34, and hereby preferably C-terminally or N-terminally positioned of the NGF antigen, further preferably C-terminally positioned of the NGF antigen. In a further embodiment, said antigen comprises, or preferably consists of, SEQ ID NO:30 or an amino acid sequence having a sequence identity of at least 90%, preferably of at least 92%, further preferably of at least 95%, and further preferably of at least 98% amino acid sequence identity with SEQ ID NO:30, and wherein said NGF antigen further comprises a polyhistidine-tag of at 4, 6, 8, or 10 consecutive histidine residues, preferably 6 consecutive histidine residues consisting of SEQ ID NO:34, and hereby preferably C-terminally or N-terminally positioned of the NGF antigen, further preferably C-terminally positioned of the NGF antigen. In a further preferred embodiment, said antigen comprises, or preferably consists of, SEQ ID NO:30 or an amino acid sequence having a sequence identity of at least 90%, preferably of at least 92%, further preferably of at least 95%, and again further preferably of at least 98% amino acid sequence identity with SEQ ID NO:30, and wherein said NGF antigen further comprises a polyhistidine-tag of 6 consecutive histidine residues consisting of SEQ ID NO:34, and preferably C-terminally or N-terminally positioned of the NGF antigen, further preferably C-terminally positioned of the NGF antigen. In a further preferred embodiment, said antigen comprises, or preferably consists of, SEQ ID NO:30 or an amino acid sequence having a sequence identity of at least 90%, preferably of at least 92%, further preferably of at least 95%, and again further preferably of at least 98% amino acid sequence identity with SEQ ID NO:30, and wherein said NGF antigen further comprises a polyhistidine-tag of 6 consecutive histidine residues consisting of SEQ ID NO:34 and N-terminally positioned of the NGF antigen. In a further embodiment, said antigen comprises SEQ ID NO:30, and wherein said NGF antigen further comprises a polyhistidine-tag of at least two consecutive and at most 12 consecutive histidine residues, preferably 4, 6, 8, or 10 consecutive histidine residues, further preferably 6 consecutive histidine residues consisting of SEQ ID NO:34, and hereby preferably C-terminally or N-terminally positioned of the NGF antigen, further preferably C-terminally positioned of the NGF antigen. In a further embodiment, said antigen comprises SEQ ID NO: 30, and wherein said NGF antigen further comprises a polyhistidine-tag of 6 consecutive histidine residues consisting of SEQ ID NO:34, and preferably C-terminally or N-terminally positioned of the NGF antigen, further preferably C-terminally positioned of the NGF antigen. In a further embodiment, said antigen comprises SEQ ID NO:30, and wherein said NGF antigen further comprises a polyhistidine-tag of 6 consecutive histidine residues consisting of SEQ ID NO:34 and N-terminally positioned of the NGF antigen. In a further very preferred embodiment, said antigen consists of SEQ ID NO:30.

In a further embodiment, said antigen is feline NGF. In a further very preferred embodiment, said antigen comprises, or preferably consists of, SEQ ID NO:55 or an amino acid sequence having a sequence identity of at least 90% or at least 91%, preferably of at least 92%, at least 93% or at least 94%, further preferably of at least 95%, at least 96% or at least 97%, and again further preferably of at least 98% or at least 99% amino acid sequence identity with SEQ ID NO:55. In a further preferred embodiment, said antigen comprises SEQ ID NO:55. In a further preferred embodiment, said antigen consists of SEQ ID NO:55.

In a further embodiment, said antigen is equine NGF. In a further embodiment, said antigen comprises, or preferably consists of, SEQ ID NO:56 or an amino acid sequence having a sequence identity of at least 90% or at least 91%, preferably of at least 92%, at least 93% or at least 94%, further preferably of at least 95%, at least 96% or at least 97%, and again further preferably of at least 98% or at least 99% amino acid sequence identity with SEQ ID NO:56. In a further preferred embodiment, said antigen comprises SEQ ID NO:56. In a further very preferred embodiment, said antigen consists of SEQ ID NO:56.

In a further embodiment, said antigen is bovine NGF. In a further preferred embodiment, said antigen comprises, or preferably consists of, SEQ ID NO:57 or an amino acid sequence having a sequence identity of at least 90% or at least 91%, preferably of at least 92%, at least 93% or at least 94%, further preferably of at least 95%, at least 96% or at least 97%, and again further preferably of at least 98% or at least 99% amino acid sequence identity with SEQ ID NO:57. In a further preferred embodiment, said antigen comprises SEQ ID NO:57. In a further very preferred embodiment, said antigen consists of SEQ ID NO: 57.

In a further embodiment, said antigen is porcine NGF. In a further preferred embodiment, said antigen comprises, or preferably consists of, SEQ ID NO:58 or an amino acid sequence having a sequence identity of at least 90% or at least 91%, preferably of at least 92%, at least 93% or at least 94%, further preferably of at least 95%, at least 96% or at least 97%, and again further preferably of at least 98% or at least 99% amino acid sequence identity with SEQ ID NO:58. In a further preferred embodiment, said antigen comprises SEQ ID NO:58. In a further very preferred embodiment, said antigen consists of SEQ ID NO:58.

### Compositions to be Used According to the Invention:

Virus-like particles (VLPs) of Cucumber Mosaic Virus (CMV) suitable for the present invention include CMV VLPs known by the skilled person in the art for example from WO2016/062720 and WO2020/128037, and in references cited therein. Due to the extremely wide host range of the cucumber mosaic virus, a lot of different strains and isolates of CMV are known and the sequences of the coat proteins of said strains and isolates have been determined and are, thus, known to the skilled person in the art as well. The sequences of said coat proteins (CPs) of CMV are described in and retrievable from the known databases such as Genbank, www.dpvweb.net, or www.ncbi.nlm.nih.gov/protein/. Examples are described on pages 12 to 14 of WO2016/062720, the disclosure incorporated herein by way of reference. The CMV VLPs of the present invention comprise at least one CMV polypeptide, typically the CMV VLPs comprise 180 copies of said CMV polypeptide forming the capsid structure. Preferably, a VLP of CMV comprises said CMV polypeptide as the major, and even more preferably as the sole protein component of its capsid structure.

Preferably, a VLP of CMV comprises, or consists of, at least one, typically and preferably 180 copies of a, CMV polypeptide comprising or preferably consisting of (i) an amino acid sequence of a coat protein of CMV; or (ii) an amino acid sequence having a sequence identity of at least 90%, preferably of at least 95%, further preferably of at least 98% and again more preferably of at least 99% to said coat protein of CMV.

In a preferred embodiment, said CMV polypeptide comprises, preferably consists of, an amino acid sequence of a coat protein of CMV or a mutated amino acid sequence, wherein said mutated amino acid sequence and said coat protein of CMV show a sequence identity of at least 90%, preferably of at least 91%, 92%, 93, 94% or 95%, further preferably of at least 96%, 97% or 98% and again more preferably of at least 99%; wherein preferably said mutated amino acid sequence and said amino acid sequence to be mutated differ in least one and in at most 11, 10, 9, 8, 7, 6, 5, 4, 3, or 2 amino acid residues, and wherein further preferably these differences are selected from (i) insertion, (ii) deletion, (iii) amino acid exchange, and (iv) any combination of (i) to (iii).

In a preferred embodiment, said CMV polypeptide comprises a coat protein of CMV or an amino acid sequence having a sequence identity of at least 75% with SEQ ID NO:39.

Thus, the present invention provides for a composition for use in a method of treating a NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and wherein said VLP of CMV comprises at least one CMV polypeptide, wherein said CMV polypeptide comprises a coat protein of CMV or an amino acid sequence having a sequence identity of at least 75% with SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In another preferred embodiment, said CMV polypeptide comprises a coat protein of CMV or an amino acid sequence having a sequence identity of at least 80% with SEQ ID NO: 39. In another preferred embodiment, said CMV polypeptide comprises a coat protein of CMV or an amino acid sequence having a sequence identity of at least 85% with SEQ ID NO: 39. In another preferred embodiment, said CMV polypeptide comprises a coat protein of CMV or an amino acid sequence having a sequence identity of at least 90% with SEQ ID NO: 39. In another preferred embodiment, said CMV polypeptide comprises a coat protein of CMV or an amino acid sequence having a sequence identity of at least 92% with SEQ ID NO: 39. In another preferred embodiment, said CMV polypeptide comprises a coat protein of CMV or an amino acid sequence having a sequence identity of at least 93% with SEQ ID NO: 39. In another preferred embodiment, said CMV polypeptide comprises a coat protein of CMV or an amino acid sequence having a sequence identity of at least 95% with SEQ ID NO: 39. In another preferred embodiment, said CMV polypeptide comprises a coat protein of CMV or an amino acid sequence having a sequence identity of at least 96% with SEQ ID NO: 39. In another preferred embodiment, said CMV polypeptide comprises a coat protein of CMV or an amino acid sequence having a sequence identity of at least 97% with SEQ ID NO: 39. In another preferred embodiment, said CMV polypeptide comprises a coat protein of CMV or an amino acid sequence having a sequence identity of at least 98% with SEQ ID NO: 39. In another preferred embodiment, said CMV polypeptide comprises a coat protein of CMV or an amino acid sequence having a sequence identity of at least 99% with SEQ ID NO: 39.

In another aspect, the present invention provides for a composition for use in a method of treating a NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and wherein said VLP of CMV comprises at least one CMV polypeptide, wherein said CMV polypeptide comprises the coat protein of CMV of SEQ ID NO:39, or an amino acid sequence having a sequence identity of at least 75% with SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In a preferred embodiment, said CMV polypeptide comprises the coat protein of CMV of SEQ ID NO:39 or an amino acid sequence having a sequence identity of at least 75% with SEQ ID NO:39. In another preferred embodiment, said CMV polypeptide comprises the coat protein of CMV of SEQ ID NO:39 or an amino acid sequence having a sequence identity of at least 80% with SEQ ID NO:39. In another preferred embodiment, said CMV polypeptide comprises the coat protein of CMV of SEQ ID NO:39 or an amino acid sequence having a sequence identity of at least 85% with SEQ ID NO:39. In another preferred embodiment, said CMV polypeptide comprises the coat protein of CMV of SEQ ID NO:39 or an amino acid sequence having a sequence identity of at least 90% with SEQ ID NO:39. In another preferred embodiment, said CMV polypeptide comprises the coat protein of CMV of SEQ ID NO: 39 or an amino acid sequence having a sequence identity of at least 92% with SEQ ID NO: 39. In another preferred embodiment, said CMV polypeptide comprises the coat protein of CMV of SEQ ID NO:39 or an amino acid sequence having a sequence identity of at least 93% with SEQ ID NO:39. In another preferred embodiment, said CMV polypeptide comprises the coat protein of CMV of SEQ ID NO:39 or an amino acid sequence having a sequence identity of at least 95% with SEQ ID NO:39. In another preferred embodiment, said CMV polypeptide comprises the coat protein of CMV of SEQ ID NO:39 or an amino acid sequence having a sequence identity of at least 96% with SEQ ID NO:39. In another preferred embodiment, said CMV polypeptide comprises the coat protein of CMV of SEQ ID NO: 39 or an amino acid sequence having a sequence identity of at least 97% with SEQ ID NO: 39. In another preferred embodiment, said CMV polypeptide comprises the coat protein of CMV of SEQ ID NO:39 or an amino acid sequence having a sequence identity of at least 98% with SEQ ID NO:39. In another preferred embodiment, said CMV polypeptide comprises the coat protein of CMV of SEQ ID NO:39 or an amino acid sequence having a sequence identity of at least 99% with SEQ ID NO:39. In another preferred embodiment, said CMV polypeptide comprises, preferably consists of the coat protein of CMV of SEQ ID NO: 39.

In another aspect, the present invention provides for a composition for use in a method of treating a NGF-related disorder in canine comprising the administration of such composition to said canine, wherein the composition comprises
(a) a virus-like particle (VLP) of Cucumber Mosaic Virus (CMV), wherein said CMV VLP comprises at least one first attachment site; and wherein said VLP of CMV comprises at least one CMV polypeptide, wherein said CMV polypeptide comprises the coat protein of CMV of SEQ ID NO:39, or an amino acid sequence having a sequence identity of at least 90% with SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second attachment site. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

The immunogenicity of the NGF containing CMV VLP compositions as described herein can be further increased by adding a T helper cell epitope. Preferably, that T helper cell epitope is the T helper cell epitope derived from tetanus toxin or is a PADRE sequence. Such T helper cell epitope preferably comprises or consists of amino acid sequence of SEQ ID NO: 41 or SEQ ID NO:42. Such T helper cell epitope can be, preferably is, introduced into the CMV VLPs, in particular into the CMV polypeptide and preferably into the coat protein sequence of the CMV VLP. According to a preferred embodiment of these T-helper cell epitope containing CMV VLPs, the T helper cell epitope, preferably the T helper cell epitope derived from tetanus toxin or the PADRE sequence, replaces a N-terminal region of the CMV polypeptide. Preferably, the T helper cell epitope, preferably the T helper cell epitope derived from tetanus toxin or the PADRE sequence, replaces a N-terminal region of said CMV polypeptide, and wherein preferably said N-terminal region of said CMV polypeptide corresponds to the amino acid residues 2-12 of SEQ ID NO:39.

Thus, according to a further aspect the present invention provides for a composition for use in a method of treating a NGF-related disorder in canine comprising the administration of said composition to said canine, wherein the composition comprises,
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
(c) a T helper cell epitope; and
wherein (a) and (b) are linked through said at least one first and said at least one second attachment site.

According to a further aspect the present invention provides for a composition for use in a method of treating a NGF-related disorder in canine comprising the administration of said composition to said canine, wherein the composition comprises,
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
(c) a T helper cell epitope, wherein said T helper cell epitope is derived from tetanus toxin or is a PADRE sequence, preferably wherein said T helper cell epitope comprises or consists of amino acid sequence of SEQ ID NO:41 or SEQ ID NO:42; and
wherein (a) and (b) are linked through said at least one first and said at least one second attachment site.

According to a further aspect the present invention provides for a composition for use in a method of treating a NGF-related disorder in canine comprising the administration of said composition to said canine, wherein the composition comprises,
(a) a virus-like particle (VLP) of Cucumber Mosaic Virus (CMV), wherein said CMV VLP comprises at least one first attachment site; and wherein said CMV VLP comprises at least one CMV polypeptide;
(b) at least one antigen, wherein said antigen comprises at least one second attachment site, and wherein said antigen is a nerve growth factor (NGF) antigen; and
(c) a T helper cell epitope, preferably the T helper cell epitope derived from tetanus toxin or the PADRE sequence, preferably comprises or consists of SEQ ID NO:41 or SEQ ID NO:42, wherein said T helper cell epitope replaces a N-terminal region of the CMV polypeptide, preferably the amino acids corresponding to amino acid residues 2-12 of SEQ ID NO:39; and
wherein (a) and (b) are linked through said at least one first and said at least one second attachment site.

In another aspect, the present invention provides for a composition for use in a method of treating a NGF-related disorder in canine comprising the administration of said composition to said canine, wherein the composition comprises,
(a) a modified VLP of cucumber mosaic virus (CMV), wherein said modified VLP of CMV comprises at least one modified CMV polypeptide, wherein said at least one modified CMV polypeptide comprises, preferably consists of,
   (i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein; and
   (ii) a T helper cell epitope, wherein preferably said T helper cell epitope replaces a N-terminal region of the CMV polypeptide, preferably, the amino acids corresponding to amino acid residues 2-12 of SEQ ID NO:39;
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In a preferred embodiment, said CMV polypeptide comprises the coat protein of CMV of SEQ ID NO:39 or an amino acid sequence having a sequence identity of at least 75% with SEQ ID NO:39. In another preferred embodiment, said CMV polypeptide comprises the coat protein of CMV of SEQ ID NO:39 or an amino acid sequence having a sequence identity of at least 80% with SEQ ID NO:39. In another preferred embodiment, said CMV polypeptide comprises the coat protein of CMV of SEQ ID NO:39 or an amino acid sequence having a sequence identity of at least 85% with SEQ ID NO:39. In another preferred embodiment, said CMV polypeptide comprises the coat protein of CMV of SEQ ID NO:39 or an amino acid sequence having a sequence identity of at least 90% with SEQ ID NO:39. In another preferred embodiment, said CMV polypeptide comprises the coat protein of CMV of SEQ ID NO: 39 or an amino acid sequence having a sequence identity of at least 92% with SEQ ID NO: 39. In another preferred embodiment, said CMV polypeptide comprises the coat protein of CMV of SEQ ID NO:39 or an amino acid sequence having a sequence identity of at least 93% with SEQ ID NO:39. In another preferred embodiment, said CMV polypeptide comprises the coat protein of CMV of SEQ ID NO:39 or an amino acid sequence having a sequence identity of at least 95% with SEQ ID NO:39. In another preferred embodiment, said CMV polypeptide comprises the coat protein of CMV of SEQ ID NO:39 or an amino acid sequence having a sequence identity of at least 96% with SEQ ID NO:39. In another preferred embodiment, said CMV polypeptide comprises the coat protein of CMV of SEQ ID NO: 39 or an amino acid sequence having a sequence identity of at least 97% with SEQ ID NO: 39. In another preferred embodiment, said CMV polypeptide comprises the coat protein of CMV of SEQ ID NO:39 or an amino acid sequence having a sequence identity of at least 98% with SEQ ID NO:39. In another preferred embodiment, said CMV polypeptide comprises the coat protein of CMV of SEQ ID NO:39 or an amino acid sequence having a sequence identity of at least 99% with SEQ ID NO:39. In another preferred embodiment, said CMV polypeptide comprises, preferably consists of the coat protein of CMV of SEQ ID NO: 39.

In a further embodiment of the present invention, the T helper cell epitope is selected from TT 830-843 (SEQ ID NO:41), PADRE (SEQ ID NO:42), HA 307-319 (SEQ ID NO: 43), HBV_{nc} 50-69 (SEQ ID NO:44), CS 378-398 (SEQ ID NO:45), MT 17-31 (SEQ ID NO: 46), and TT 947-967 (SEQ ID NO:47). In a preferred embodiment, said T helper cell epitope is a T helper cell epitope derived from tetanus toxin or is a PADRE sequence. In a preferred embodiment, said T helper cell epitope is derived from a human vaccine. In a preferred embodiment, said T helper cell epitope is a T helper cell epitope derived from tetanus toxin. In a preferred embodiment, said T helper cell epitope is a PADRE sequence. In a preferred embodiment, said T helper cell epitope comprises the amino acid sequence of SEQ ID NO:41 or SEQ ID NO:42. In a very preferred embodiment, said T helper cell epitope consists of the amino acid sequence of SEQ ID NO:41 or SEQ ID NO:42. In a very preferred embodiment, said T helper cell epitope comprises the amino acid sequence of SEQ ID NO: 41. In a preferred embodiment, said T helper cell epitope consists of the amino acid sequence of SEQ ID NO:41. In a very preferred embodiment, said T helper cell epitope comprises the amino acid sequence of SEQ ID NO:42. In a very preferred embodiment, said T helper cell epitope consists of the amino acid sequence of SEQ ID NO:42.

Thus, in another aspect, the present invention provides for a composition for use in a method of treating a NGF-related disorder in canine comprising the administration of said composition to said canine, wherein the composition comprises,
(a) a modified VLP of cucumber mosaic virus (CMV), wherein said modified VLP of CMV comprises at least one modified CMV polypeptide, wherein said at least one modified CMV polypeptide comprises, preferably consists of,
   (i) a CMV polypeptide, wherein said CMV polypeptide comprises, preferably consists of, the coat protein of CMV of SEQ ID NO:39, or an amino acid sequence having a sequence identity of at least 75%, preferably of at least 80%, further preferably of at least 85%, again further preferably of at least 90%, again further preferably of at least 92%, again further preferably of at least 95%, again further preferably of at least 98% with said coat protein; and
   (ii) a T helper cell epitope, preferably, wherein said T helper cell epitope replaces a N-terminal region of said CMV polypeptide, further preferably said N-terminal region of said CMV polypeptide corresponds to amino acids 2-12 of SEQ ID NO:39, further preferably said T helper cell epitope is derived from tetanus toxin or is a PADRE sequence, even more preferably, said T helper cell epitope comprises, further preferably consists of, the amino acid sequence of SEQ ID NO:41 or SEQ ID NO:42;
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In a preferred embodiment, said T helper cell epitope is derived from tetanus toxin or is a PADRE sequence, wherein said T helper cell epitope comprises, preferably consists of, the amino acid sequence of SEQ ID NO:41 or SEQ ID NO:42. In a further preferred embodiment, said CMV polypeptide is a coat protein of CMV or an amino acid sequence having a sequence identity of at least 90%, preferably at least 92%, further preferably at least 95%, and again further preferably at least 98% with SEQ ID NO:39, and wherein said T helper cell epitope is derived from tetanus toxin or is a PADRE sequence, wherein said T helper cell epitope comprises, preferably consists of, the amino acid sequence of SEQ ID NO: 41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

Thus, in another aspect, the present invention provides for a composition for use in a method of treating a NGF-related disorder in canine comprising the administration of said composition to said canine, wherein the composition comprises,
(a) a modified VLP of cucumber mosaic virus (CMV), wherein said modified VLP of CMV comprises at least one modified CMV polypeptide, wherein said at least one modified CMV polypeptide comprises, preferably consists of,
   i. a CMV polypeptide, wherein said CMV polypeptide comprises, preferably consists of, the coat protein of CMV of SEQ ID NO:39, or an amino acid sequence having a sequence identity of at least 90%, preferably of at least 92%, further preferably of at least 95%, again further preferably of at least 98% with SEQ ID NO:39; and
   ii. a T helper cell epitope, wherein said T helper cell epitope replaces a N-terminal region of said CMV polypeptide, and wherein said N-terminal region of said CMV polypeptide corresponds to amino acids 2-12 of SEQ ID NO: 39, and wherein preferably said T helper cell epitope is derived from tetanus toxin or is a PADRE sequence, wherein very preferably, said T helper cell epitope comprises, again further preferably consists of, the amino acid sequence of SEQ ID NO:41 or SEQ ID NO:42.
(b) at least one antigen, wherein said antigen comprises at least one second attachment site, and wherein said antigen is a nerve growth factor (NGF) antigen; and
wherein (a) and (b) are linked through said at least one first and said at least one second attachment site.

In a preferred embodiment, said modified CMV polypeptide comprises, preferably consists of, the amino acid sequence of SEQ ID NO:5 or of SEQ ID NO:48. In a very preferred embodiment, said modified CMV polypeptide comprises, preferably consists of, the amino acid sequence of SEQ ID NO:5.

Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

There are different ways of performing the linkage between the CMV VLP and the NGF antigen. The NGF antigen can be linked to the CMV VLP via the attachment sites by peptide bonds, preferably by genetic fusion. For instance, the nucleotide sequence coding for the NGF antigen can be cloned in frame within the nucleotide sequence coding for the CMV VLP. Alternatively, the NGF antigen can be linked to the CMV VLPs via chemical coupling between the attachment sites for instance by at least one non-peptide bond.

In a preferred embodiment, said CMV VLP and said at least one NGF antigen are linked through said at least one first and said at least one second attachment site via at least one covalent bond. In a preferred embodiment, said CMV VLP and said at least one NGF antigen are linked through said at least one first and said at least one second attachment site via exclusively covalent bonds.

In a further preferred embodiment, said CMV VLP and said at least one NGF antigen are linked through said at least one first and said at least one second attachment site via at least one covalent peptide bond. In a further preferred embodiment, said CMV VLP and said at least one NGF antigen are linked through said at least one first and said at least one second attachment site via exclusively covalent peptide bonds.

In another preferred embodiment, said CMV VLP and said at least one NGF antigen are linked through said at least one first and said at least one second attachment site via at least one covalent non-peptide bond. In another preferred embodiment, said CMV VLP and said at least one NGF antigen are linked through said at least one first and said at least one second attachment site via exclusively covalent non-peptide bonds.

Thus, according to a further aspect, the present invention provides for a composition for use in a method of treating an NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent peptide bond, preferably by the way of fusion.

In a further aspect, the present invention provides a composition for use in a method of treating an NGF-related disorder in canine comprising the administration of said composition to said canine, wherein the composition comprises a modified VLP of CMV, and wherein said modified VLP of CMV comprises at least one antigenic CMV fusion polypeptide, wherein said at least one antigenic CMV fusion polypeptide comprises, preferably consists of,
(i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
(ii) an antigenic NGF polypeptide, wherein said antigenic NGF polypeptide is inserted into said CMV polypeptide, wherein preferably said insertion of said antigenic NGF polypeptide is between amino acid residues of said CMV polypeptide corresponding to amino acid residues of position 84 and position 85 of SEQ ID NO:39; and
(iii) optionally, a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39.

According to another aspect, the present invention provides a composition for use in a method of treating an NGF-related disorder in canine comprising the administration of said composition to said canine, wherein the composition comprises a modified VLP of CMV, and wherein said modified VLP of CMV comprises at least one antigenic CMV fusion polypeptide, wherein said at least one antigenic CMV fusion polypeptide comprises, preferably consists of,
(a) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
(b) an antigenic NGF polypeptide, wherein said antigenic NGF polypeptide is inserted into said CMV polypeptide, wherein preferably said insertion of said antigenic NGF polypeptide is between amino acid residues of said CMV polypeptide corresponding to amino acid residues of position 84 and position 85 of SEQ ID NO:39; and
(c) a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39.

Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In another aspect, the present invention provides a composition for use in a method of treating an NGF-related disorder in canine comprising the administration of said composition to said canine, wherein the composition comprises a modified VLP of CMV, and wherein said modified VLP of CMV comprises at least one antigenic CMV fusion polypeptide, wherein said at least one antigenic CMV fusion polypeptide comprises, preferably consists of,
(i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
(ii) an antigenic NGF polypeptide, wherein said antigenic NGF polypeptide is inserted into said CMV polypeptide, wherein said insertion of said antigenic NGF polypeptide is between amino acid residues of said CMV polypeptide corresponding to amino acid residues of position 84 and position 85 of SEQ ID NO: 39; and
(iii) optionally a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39.

In another aspect, the present invention provides a composition for use in a method of treating an NGF-related disorder in canine comprising the administration of said composition to said canine, wherein the composition comprises a modified VLP of CMV, and wherein said modified VLP of CMV comprises at least one antigenic CMV fusion polypeptide, wherein said at least one antigenic CMV fusion polypeptide comprises, preferably consists of,
(a) CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, again further preferably of at least 95%, further preferably of at least 98% with said coat protein with SEQ ID NO:39; and
(b) an antigenic NGF polypeptide, wherein said antigenic NGF polypeptide is inserted into said CMV polypeptide, wherein said insertion of said antigenic NGF polypeptide is between amino acid residues of said CMV polypeptide corresponding to amino acid residues of position 84 and position 85 of SEQ ID NO: 39; and
(c) a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39.

Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to a further aspect, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond.

A CMV VLP is typically a macromolecular assembly composed of viral coat protein which typically comprises 180 protein subunits per VLP. Typically and preferably, the interactions of these subunits lead to the formation of VLPs with an inherent repetitive organization allowing the presentation of multiple copies of NGF antigens. Such coat protein is for example the coat protein as encoded by amino acid sequence SEQ ID NO:39 or having at least 75% sequence identity with SEQ ID NO:39.

Thus, according to a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one CMV polypeptide, wherein said at least one CMV polypeptide comprises, preferably consists of, a CMV polypeptide, wherein said CMV polypeptide comprises a coat protein of CMV or an amino acid sequence having a sequence identity of at least 75% with SEQ ID NO:39; and
(b) at least one antigen, wherein said antigen comprises at least one second attachment site, and wherein said NGF antigen is a nerve growth factor (NGF) antigen; and
wherein (a) and (b) are linked through said at least one first and said at least one second attachment site via at least one covalent non-peptide bond. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one modified CMV polypeptide, wherein said at least one modified CMV polypeptide comprises, preferably consists of,
   (i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
   (ii) optionally, a T helper cell epitope; and
(b) a wherein said antigen comprises at least one second attachment site, and wherein said NGF antigen is a nerve growth factor (NGF) antigen; and
wherein (a) and (b) are linked through said at least one first and said at least one second attachment site via at least one covalent non-peptide bond.

In a further aspect, the present invention provides for a composition for use in a method of treating a NGF-related disorder, in canine comprising the administration of said composition to said canine, wherein the composition comprises
(c) a modified VLP of CMV, wherein said modified VLP of CMV comprises at least one first attachment site, and wherein said modified VLP of CMV comprises at least one modified CMV polypeptide, wherein said at least one modified CMV polypeptide comprises, preferably consists of,
   (i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
   (ii) a T helper cell epitope; and
(d) a wherein said antigen comprises at least one second attachment site, and wherein said NGF antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a preferred embodiment, said CMV polypeptide comprises the coat protein of CMV of SEQ ID NO:39 or an amino acid sequence having a sequence identity of at least 75% with SEQ ID NO:39. In another preferred embodiment, said CMV polypeptide comprises the coat protein of CMV of SEQ ID NO:39 or an amino acid sequence having a sequence identity of at least 80% with SEQ ID NO:39. In another preferred embodiment, said CMV polypeptide comprises the coat protein of CMV of SEQ ID NO:39 or an amino acid sequence having a sequence identity of at least 85% with SEQ ID NO:39. In another preferred embodiment, said CMV polypeptide comprises the coat protein of CMV of SEQ ID NO:39 or an amino acid sequence having a sequence identity of at least 90% with SEQ ID NO:39. In another preferred embodiment, said CMV polypeptide comprises the coat protein of CMV of SEQ ID NO: 39 or an amino acid sequence having a sequence identity of at least 92% with SEQ ID NO: 39. In another preferred embodiment, said CMV polypeptide comprises the coat protein of CMV of SEQ ID NO:39 or an amino acid sequence having a sequence identity of at least 93% with SEQ ID NO:39. In another preferred embodiment, said CMV polypeptide comprises the coat protein of CMV of SEQ ID NO:39 or an amino acid sequence having a sequence identity of at least 95% with SEQ ID NO:39. In another preferred embodiment, said CMV polypeptide comprises the coat protein of CMV of SEQ ID NO:39 or an amino acid sequence having a sequence identity of at least 96% with SEQ ID NO:39. In another preferred embodiment, said CMV polypeptide comprises the coat protein of CMV of SEQ ID NO: 39 or an amino acid sequence having a sequence identity of at least 97% with SEQ ID NO: 39. In another preferred embodiment, said CMV polypeptide comprises the coat protein of CMV of SEQ ID NO:39 or an amino acid sequence having a sequence identity of at least 98% with SEQ ID NO:39. In another preferred embodiment, said CMV polypeptide comprises the coat protein of CMV of SEQ ID NO:39 or an amino acid sequence having a sequence identity of at least 99% with SEQ ID NO:39. In another preferred embodiment, said CMV polypeptide comprises, preferably consists of the coat protein of CMV of SEQ ID NO: 39. In a preferred embodiment, the T helper cell epitope is selected from TT 830-843 (SEQ ID NO:41), PADRE (SEQ ID NO:42), HA 307-319 (SEQ ID NO:43), HBV_{nc} 50-69 (SEQ ID NO:44), CS 378-398 (SEQ ID NO:45), MT 17-31 (SEQ ID NO:46), and TT 947-967 (SEQ ID NO:47). In a preferred embodiment, said T helper cell epitope is a T helper cell epitope derived from tetanus toxin or is a PADRE sequence. In a preferred embodiment, said T helper cell epitope is derived from a human vaccine. In a preferred embodiment, said T helper cell epitope is a T helper cell epitope derived from tetanus toxin. In a preferred embodiment, said T helper cell epitope is a PADRE sequence. In a preferred embodiment, said T helper cell epitope comprises the amino acid sequence of SEQ ID NO:41 or SEQ ID NO: 42. In a very preferred embodiment, said T helper cell epitope consists of the amino acid sequence of SEQ ID NO:41 or SEQ ID NO:42. In a very preferred embodiment, said T helper cell epitope comprises the amino acid sequence of SEQ ID NO:41. In a preferred embodiment, said T helper cell epitope consists of the amino acid sequence of SEQ ID NO: 41. In a very preferred embodiment, said T helper cell epitope comprises the amino acid sequence of SEQ ID NO:42. In a very preferred embodiment, said T helper cell epitope consists of the amino acid sequence of SEQ ID NO:42. In a very preferred embodiment, said modified CMV polypeptide comprises, preferably consists of, the amino acid sequence of SEQ ID NO:5 or of SEQ ID NO:48. In a very preferred embodiment, said modified CMV polypeptide comprises, preferably consists of, the amino acid sequence of SEQ ID NO:5.

In a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one modified CMV polypeptide, wherein said at least one modified CMV polypeptide comprises, preferably consists of,
   (i) a CMV polypeptide, wherein said CMV polypeptide comprises, preferably consists of, a coat protein of CMV or an amino acid sequence having a sequence identity of at least 75%, preferably of at least 80%, further preferably of at least 85%, further preferably of at least 90%, further preferably of at least 92%, further preferably of at least 95%, further preferably of at least 98% with said coat protein with SEQ ID NO:39; and
   (ii) a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39, and
(b) a wherein said antigen comprises at least one second attachment site, and wherein said NGF antigen is a nerve growth factor (NGF) antigen; and
wherein (a) and (b) are linked through said at least one first and said at least one second attachment site via at least one covalent non-peptide bond. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a another aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, preferably wherein said polypeptide is inserted between any amino acid residue of said CMV polypeptide corresponding to any amino acid residue between position 75 and position 85 of SEQ ID NO:39.
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

Thus, in another aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:39 and
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a preferred embodiment, said chimeric CMV polypeptide further comprises a T helper cell epitope, preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42, and wherein preferably said T helper cell epitope replaces a N-terminal region of said CMV polypeptide, preferably said N-terminal region of said CMV polypeptide corresponds to amino acids 2-12 of SEQ ID NO:39. In a further very preferred embodiment, said CMV polypeptide is a coat protein of CMV or an amino acid sequence having a sequence identity of at least 90%, preferably at least 92%, further preferably at least 95%, and further preferably at least 98% with SEQ ID NO:39.

Thus, in another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, and
   (iii) a T helper cell epitope, preferably wherein said T helper cell epitope replaces a N-terminal region of said CMV polypeptide; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:39 and
   (iii) a T helper cell epitope, preferably wherein said T helper cell epitope replaces a N-terminal region of said CMV polypeptide; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. According to another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, and
   (iii) a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:39 and
   (iii) a T helper cell epitope, preferably wherein said T helper cell epitope is derived from tetanus toxin or is a PADRE sequence, further preferably, wherein said T helper cell epitope comprises, preferably consists of, the amino acid sequence of SEQ ID NO:41 or SEQ ID NO:42. Preferably said T helper cell epitope replaces a N-terminal region of said CMV polypeptide, and wherein further preferably said N-terminal region of said CMV polypeptide corresponds to amino acids 2-12 of SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a further very preferred embodiment, said stretch of consecutive negative amino acids comprises, preferably consists of SEQ ID NO:1 or SEQ ID NO:2.

Thus, according to a further aspect, the present invention provides for a composition for use in a method of treating a NGF-related disorder in a canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39; and
   (ii) a polypeptide comprising, preferably consisting of, a stretch of consecutive negative amino acids, wherein said negative amino acids are independently selected from aspartic acid or glutamic acid, and wherein said stretch of consecutive negative amino acids comprises, preferably consists of, SEQ ID NO: 1 or SEQ ID NO:2;
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

Thus, according to a further aspect, the present invention provides for a composition for use in a method of treating a NGF-related disorder in a canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39; and
   (ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:39, and wherein said stretch of consecutive negative amino acids comprises, preferably consists of, SEQ ID NO: 1 or SEQ ID NO:2;
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to another aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39;
   (ii) a polypeptide comprising, preferably consisting of, a stretch of consecutive negative amino acids, wherein said negative amino acids are independently selected from aspartic acid or glutamic acid, and wherein said stretch of consecutive negative amino acids comprises, preferably consists of SEQ ID NO: 1 or SEQ ID NO:2; and
   (iii) a T helper cell epitope, preferably wherein said T helper cell epitope replaces a N-terminal region of said CMV polypeptide and/or preferably wherein said T helper cell epitope is derived from tetanus toxin or is a PADRE sequence, further preferably, wherein said T helper cell epitope comprises, preferably consists of, the amino acid sequence of SEQ ID NO: 41 or SEQ ID NO:42; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope replaces said N-terminal region of said CMV polypeptide corresponds to amino acids 2-12 of SEQ ID NO:39. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to another aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39;
   (ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:39, and wherein said stretch of consecutive negative amino acids comprises, preferably consists of SEQ ID NO:1 or SEQ ID NO: 2; and
   (iii) a T helper cell epitope, preferably wherein said T helper cell epitope replaces a N-terminal region of said CMV polypeptide and/or preferably wherein said T helper cell epitope is derived from tetanus toxin or is a PADRE sequence, further preferably, wherein said T helper cell epitope comprises, preferably consists of, the amino acid sequence of SEQ ID NO: 41 or SEQ ID NO:42; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope replaces said N-terminal region of said CMV polypeptide corresponds to amino acids 2-12 of SEQ ID NO:39. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1 or an amino acid linker (GS*-linker) comprising at least one Gly, at least one Ser, and at least one amino acid selected from Thr, Ala, Lys, and Cys. In a further very preferred embodiment, said first amino acid linker comprises, preferably consists of, SEQ ID NO:8.

In a further very preferred embodiment, said second amino acid linker comprises, preferably consists of, SEQ ID NO:4 or SEQ ID NO:9. In a further very preferred embodiment, said polypeptide comprises SEQ ID NO:49, SEQ ID NO:50 or SEQ ID NO:51. In a further very preferred embodiment, said polypeptide consists of SEQ ID NO:49, SEQ ID NO:50 or SEQ ID NO: 51. In a further very preferred embodiment, said polypeptide comprising, preferably consisting of, a stretch of consecutive negative amino acids is inserted between amino acid residues of said CMV polypeptide corresponding to amino acid residues of position 84 and position 85 of SEQ ID NO:39.

Thus, in a further aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39; and
   (ii) a polypeptide comprising, preferably consisting of, a stretch of consecutive negative amino acids, wherein said negative amino acids are independently selected from aspartic acid or glutamic acid, and wherein said polypeptide is preferably inserted between any amino acid residue of said CMV polypeptide corresponding to any amino acid residue between position 75 and position 85 of SEQ ID NO:39, and wherein said polypeptide comprises, preferably consists of, SEQ ID NO:49, SEQ ID NO:50 or SEQ ID NO:51, and wherein preferably said polypeptide is inserted between amino acid residues of said CMV polypeptide corresponding to amino acid residues of position 84 and position 85 of SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39, wherein preferably said CMV polypeptide is a coat protein of CMV or an amino acid sequence having a sequence identity of at least 90%, preferably 95% with SEQ ID NO:39;
   (ii) a polypeptide comprising, preferably consisting of, a stretch of consecutive negative amino acids, wherein said negative amino acids are independently selected from aspartic acid or glutamic acid, and wherein said polypeptide is preferably inserted between any amino acid residue of said CMV polypeptide corresponding to any amino acid residue between position 75 and position 85 of SEQ ID NO:39, and wherein said polypeptide comprises, preferably consists of, SEQ ID NO:49, SEQ ID NO:50 or SEQ ID NO:51; and wherein preferably said polypeptide is inserted between amino acid residues of said CMV polypeptide corresponding to amino acid residues of position 84 and position 85 of SEQ ID NO:39;
   (iii) a T helper cell epitope, preferably wherein said T helper cell epitope replaces a N-terminal region of said CMV polypeptide and/or preferably wherein said T helper cell epitope is derived from tetanus toxin or is a PADRE sequence, further preferably, wherein said T helper cell epitope comprises, preferably consists of, the amino acid sequence of SEQ ID NO: 41 or SEQ ID NO:42; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope replaces said N-terminal region of said CMV polypeptide corresponds to amino acids 2-12 of SEQ ID NO:39. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42.

In a further very preferred embodiment, said CMV polypeptide comprises, preferably consists of, the amino acid sequence of SEQ ID NO:5, SEQ ID NO:39 or SEQ ID NO: 48, wherein said polypeptide comprising said stretch of consecutive negative amino acids is inserted between amino acid residues of position 88 and position 89 of said SEQ ID NO: 5, between amino acid residues of position 84 and position 85 of SEQ ID NO:39, or between amino acid residues of position 86 and position 87 of SEQ ID NO:48. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

Thus, in a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises a coat protein of CMV or an amino acid sequence having a sequence identity of at least 75% with SEQ ID NO:39;
   (ii) a polypeptide comprising a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:39; and wherein said CMV polypeptide comprises, preferably consists of, the amino acid sequence of SEQ ID NO:5, SEQ ID NO:39 or SEQ ID NO:48, and wherein said polypeptide comprising said stretch of consecutive negative amino acids is inserted between amino acid residues of position 88 and position 89 of said SEQ ID NO: 5, between amino acid residues of position 84 and position 85 of SEQ ID NO: 39, or between amino acid residues of position 86 and position 87 of SEQ ID NO: 48; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

According to a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of,
   (i) a CMV polypeptide, wherein said CMV polypeptide comprises, preferably consists of, the amino acid sequence of SEQ ID NO:5, SEQ ID NO:39 or SEQ ID NO:48;
   (ii) a polypeptide comprising a stretch of consecutive negative amino acids, wherein said negative amino acids are independently selected from aspartic acid or glutamic acid, and wherein said polypeptide comprising said stretch of consecutive negative amino acids is inserted between amino acid residues of position 88 and position 89 of said SEQ ID NO:5, between amino acid residues of position 84 and position 85 of SEQ ID NO:39, or between amino acid residues of position 86 and position 87 of SEQ ID NO:48; and
   (iii) a T helper cell epitope, preferably wherein said T helper cell epitope replaces a N-terminal region of said CMV polypeptide and/or preferably wherein said T helper cell epitope is derived from tetanus toxin or is a PADRE sequence, further preferably, wherein said T helper cell epitope comprises, preferably consists of, the amino acid sequence of SEQ ID NO: 41 or SEQ ID NO:42; and
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond. Preferably said T helper cell epitope replaces said N-terminal region of said CMV polypeptide corresponds to amino acids 2-12 of SEQ ID NO:39. Preferably said T helper cell epitope is derived from tetanus toxin or is a PADRE sequence, preferably, said T helper cell epitope comprises, further preferably consists of, the amino acid sequence of SEQ ID NO:41 or SEQ ID NO:42. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

The herein described and disclosed embodiments, preferred embodiments and very preferred embodiments should apply to all aspects and other embodiments, preferred embodiments and very preferred embodiments irrespective of whether is specifically referred to or its repetition is avoided for the sake of conciseness.

In a preferred embodiment, said CMV polypeptide comprises, preferably consists of, an amino acid sequence of a coat protein of CMV or a mutated amino acid sequence, wherein said mutated amino acid sequence and said coat protein of CMV show a sequence identity of at least 90%, preferably of at least 91%, 92%, 93, 94% or 95%, further preferably of at least 96%, 97% or 98% and more preferably of at least 99%; wherein preferably said mutated amino acid sequence and said amino acid sequence to be mutated differ in least one and in at most 11, 10, 9, 8, 7, 6, 5, 4, 3, or 2 amino acid residues, and wherein further preferably these differences are selected from (i) insertion, (ii) deletion, (iii) amino acid exchange, and (iv) any combination of (i) to (iii).

In a preferred embodiment, said CMV polypeptide comprises a coat protein of CMV or an amino acid sequence having a sequence identity of at least 75% with SEQ ID NO:39. In another preferred embodiment, said CMV polypeptide comprises a coat protein of CMV or an amino acid sequence having a sequence identity of at least 80% with SEQ ID NO:39. In another preferred embodiment, said CMV polypeptide comprises a coat protein of CMV or an amino acid sequence having a sequence identity of at least 85% with SEQ ID NO:39.

In another preferred embodiment, said CMV polypeptide comprises a coat protein of CMV or an amino acid sequence having a sequence identity of at least 90% with SEQ ID NO:39. In another preferred embodiment, said CMV polypeptide comprises a coat protein of CMV or an amino acid sequence having a sequence identity of at least 92% with SEQ ID NO:39. In another preferred embodiment, said CMV polypeptide comprises a coat protein of CMV or an amino acid sequence having a sequence identity of at least 93% with SEQ ID NO:39. In another preferred embodiment, said CMV polypeptide comprises a coat protein of CMV or an amino acid sequence having a sequence identity of at least 95% with SEQ ID NO:39. In another preferred embodiment, said CMV polypeptide comprises a coat protein of CMV or an amino acid sequence having a sequence identity of at least 96% with SEQ ID NO:39. In another preferred embodiment, said CMV polypeptide comprises a coat protein of CMV or an amino acid sequence having a sequence identity of at least 97% with SEQ ID NO:39. In another preferred embodiment, said CMV polypeptide comprises a coat protein of CMV or an amino acid sequence having a sequence identity of at least 98% with SEQ ID NO:39. In another preferred embodiment, said CMV polypeptide comprises a coat protein of CMV or an amino acid sequence having a sequence identity of at least 99% with SEQ ID NO:39.

In a preferred embodiment, said CMV polypeptide consists of a coat protein of CMV or an amino acid sequence having a sequence identity of at least 75% with SEQ ID NO:39. In another preferred embodiment, said CMV polypeptide consists of a coat protein of CMV or an amino acid sequence having a sequence identity of at least 80% with SEQ ID NO:39. In another preferred embodiment, said CMV polypeptide consists of a coat protein of CMV or an amino acid sequence having a sequence identity of at least 85% with SEQ ID NO:39. In another preferred embodiment, said CMV polypeptide consists of a coat protein of CMV or an amino acid sequence having a sequence identity of at least 90% with SEQ ID NO:39. In another preferred embodiment, said CMV polypeptide consists of a coat protein of CMV or an amino acid sequence having a sequence identity of at least 92% with SEQ ID NO:39. In another preferred embodiment, said CMV polypeptide consists of a coat protein of CMV or an amino acid sequence having a sequence identity of at least 93% with SEQ ID NO:39. In another preferred embodiment, said CMV polypeptide consists of a coat protein of CMV or an amino acid sequence having a sequence identity of at least 95% with SEQ ID NO:39. In another preferred embodiment, said CMV polypeptide consists of a coat protein of CMV or an amino acid sequence having a sequence identity of at least 96% with SEQ ID NO:39. In another preferred embodiment, said CMV polypeptide consists of a coat protein of CMV or an amino acid sequence having a sequence identity of at least 97% with SEQ ID NO:39. In another preferred embodiment, said CMV polypeptide consists of a coat protein of CMV or an amino acid sequence having a sequence identity of at least 98% with SEQ ID NO:39. In another preferred embodiment, said CMV polypeptide consists of a coat protein of CMV or an amino acid sequence having a sequence identity of at least 99% with SEQ ID NO:39.

In a preferred embodiment, said CMV polypeptide is a coat protein of CMV or an amino acid sequence having a sequence identity of at least 75%, preferably 85% with SEQ ID NO: 39. In a preferred embodiment, said CMV polypeptide is a coat protein of CMV or an amino acid sequence having a sequence identity of at least 90%, preferably 95% with SEQ ID NO:39. In a preferred embodiment, said CMV polypeptide is a coat protein of CMV with SEQ ID NO:39. In a preferred embodiment, said coat protein of CMV comprises SEQ ID NO: 39. In a preferred embodiment, said coat protein of CMV consists of SEQ ID NO:39. In a preferred embodiment, said CMV polypeptide comprises a coat protein of CMV. In a preferred embodiment, said CMV polypeptide consists of a coat protein of CMV. In a preferred embodiment, said CMV polypeptide comprises a coat protein of CMV, wherein said coat protein of CMV comprises SEQ ID NO:39. In a preferred embodiment, said CMV polypeptide comprises a coat protein of CMV, wherein said coat protein of CMV consists of SEQ ID NO:39. In a preferred embodiment, said CMV polypeptide consists of a coat protein of CMV, wherein said coat protein of CMV consists of SEQ ID NO:39.

In a preferred embodiment, said CMV polypeptide comprises SEQ ID NO:40 or an amino acid sequence region, wherein said amino acid sequence region has a sequence identity of at least 75% with SEQ ID NO:40. In a preferred embodiment, said CMV polypeptide comprises SEQ ID NO:40 or an amino acid sequence region, wherein said amino acid sequence region has a sequence identity of at least 80% with SEQ ID NO:40. In a preferred embodiment, said CMV polypeptide comprises SEQ ID NO:40 or an amino acid sequence region, wherein said amino acid sequence region has a sequence identity of at least 85% with SEQ ID NO:40. In a preferred embodiment, said CMV polypeptide comprises SEQ ID NO:40 or an amino acid sequence region, wherein said amino acid sequence region has a sequence identity of at least 90% with SEQ ID NO:40. In a preferred embodiment, said CMV polypeptide comprises SEQ ID NO:40 or an amino acid sequence region, wherein said amino acid sequence region has a sequence identity of at least 95% with SEQ ID NO:40. In a preferred embodiment, said CMV polypeptide comprises SEQ ID NO:40 or an amino acid sequence region, wherein said amino acid sequence region has a sequence identity of at least 98% with SEQ ID NO:40. In a preferred embodiment, said CMV polypeptide comprises SEQ ID NO:40 or an amino acid sequence region, wherein said amino acid sequence region has a sequence identity of at least 99% with SEQ ID NO:40.

In a preferred embodiment, said CMV polypeptide comprises, or preferably consists of, (i) an amino acid sequence of a coat protein of CMV, wherein said amino acid sequence comprises, or preferably consists of, SEQ ID NO:39; or (ii) an amino acid sequence having a sequence identity of at least 90% of SEQ ID NO:39; and wherein said amino sequence as defined in (i) or (ii) comprises SEQ ID NO:40 or an amino acid sequence region, wherein said amino acid sequence region has a sequence identity of at least 90% with SEQ ID NO:40. In a preferred embodiment, said CMV polypeptide comprises, or preferably consists of, (i) an amino acid sequence of a coat protein of CMV, wherein said amino acid sequence comprises, or preferably consists of, SEQ ID NO:39; or (ii) an amino acid sequence having a sequence identity of at least 95% of SEQ ID NO:39; and wherein said amino sequence as defined in (i) or (ii) comprises SEQ ID NO:40 or an amino acid sequence region, wherein said amino acid sequence region has a sequence identity of at least 95% with SEQ ID NO:40. In a preferred embodiment, said CMV polypeptide comprises, or preferably consists of, (i) an amino acid sequence of a coat protein of CMV, wherein said amino acid sequence comprises, or preferably consists of, SEQ ID NO:39; or (ii) an amino acid sequence having a sequence identity of at least 90% of SEQ ID NO:39; and wherein said amino sequence as defined in (i) or (ii) comprises SEQ ID NO:40.

In a preferred embodiment, the number of amino acids of said N-terminal region replaced is equal to or lower than the number of amino acids of which said T helper cell epitope consists. In a preferred embodiment, said replaced N-terminal region of said CMV polypeptide consists of 5 to 15 consecutive amino acids. In a preferred embodiment, said replaced N-terminal region of said CMV polypeptide consists of 9 to 14 consecutive amino acids. In a preferred embodiment, said replaced N-terminal region of said CMV polypeptide consists of 11 to 13 consecutive amino acids. In a preferred embodiment, said N-terminal region of said CMV polypeptide corresponds to amino acids 2-12 of SEQ ID NO:39. In a preferred embodiment, said N-terminal region of said CMV polypeptide comprises amino acids 2-12 of SEQ ID NO:39. In a preferred embodiment, said N-terminal region of said CMV polypeptide consists of amino acids 2-12 of SEQ ID NO:39. In a preferred embodiment, said T helper cell epitope consists of at most 20 amino acids.

In a preferred embodiment of the present invention, the Th cell epitope is selected from TT 830-843 (SEQ ID NO:41), PADRE (SEQ ID NO:42), HA 307-319 (SEQ ID NO:43), HB V_{nc} 50-69 (SEQ ID NO:44), CS 378-398 (SEQ ID NO:45), MT 17-31 (SEQ ID NO:46), and TT 947-967 (SEQ ID NO:47). In a preferred embodiment, said Th cell epitope is a Th cell epitope derived from tetanus toxin or is a PADRE sequence. In a preferred embodiment, said T helper cell epitope is derived from a human vaccine. In a preferred embodiment, said Th cell epitope is a Th cell epitope derived from tetanus toxin. In a preferred embodiment, said Th cell epitope is a PADRE sequence. In a preferred embodiment, said Th cell epitope comprises the amino acid sequence of SEQ ID NO:41 or SEQ ID NO:42. In a very preferred embodiment, said Th cell epitope consists of the amino acid sequence of SEQ ID NO:41 or SEQ ID NO:42. In a very preferred embodiment, said Th cell epitope comprises the amino acid sequence of SEQ ID NO:41. In a preferred embodiment, said Th cell epitope consists of the amino acid sequence of SEQ ID NO:41. In a very preferred embodiment, said Th cell epitope comprises the amino acid sequence of SEQ ID NO:42. In a very preferred embodiment, said Th cell epitope consists of the amino acid sequence of SEQ ID NO:42.

In a preferred embodiment, said CMV polypeptide comprises, or preferably consists of, an amino acid sequence of a coat protein of CMV, wherein said amino acid sequence comprises, or preferably consists of, SEQ ID NO:39 or an amino acid sequence having a sequence identity of at least 95% of SEQ ID NO:39; and wherein said amino sequence comprises SEQ ID NO:40, and wherein said T helper cell epitope replaces the N-terminal region of said CMV polypeptide, and wherein said replaced N-terminal region of said CMV polypeptide consists of 11 to 13 consecutive amino acids, preferably of 11 consecutive amino acids, and wherein further preferably said N-terminal region of said CMV polypeptide corresponds to amino acids 2-12 of SEQ ID NO:39. In a preferred embodiment, said chimeric CMV polypeptide comprises, preferably consists of, the amino acid sequence of SEQ ID NO:5, in which said polypeptide is inserted between any amino acid residue of said CMV polypeptide corresponding to any amino acid residue between position 75 and position 85 of SEQ ID NO:39. In another preferred embodiment, said chimeric CMV polypeptide comprises the amino acid sequence of SEQ ID NO:48, in which said polypeptide is inserted between any amino acid residue of said CMV polypeptide corresponding to any amino acid residue between position 75 and position 85 of SEQ ID NO:39.

In a preferred embodiment, said stretch of consecutive negative amino acids has a length of 3 and less than 12 amino acids. In a preferred embodiment, said stretch of consecutive negative amino acids has a length of 3 to 10 amino acids. In a further preferred embodiment, said stretch of consecutive negative amino acids has a length of 3, 4, 5, 6, 7, 8, 9 or 10 amino acids. In a further preferred embodiment, said stretch of consecutive negative amino acids has a length of 3 to 9 amino acids. In a further preferred embodiment, said stretch of consecutive negative amino acids has a length of 3 to 8 amino acids. In a further preferred embodiment, said stretch of consecutive negative amino acids has a length of 4 to 9 amino acids. In a further preferred embodiment, said stretch of consecutive negative amino acids has a length of 4 to 8 amino acids. In a further preferred embodiment, said stretch of consecutive negative amino acids has a length of 4, 5, 6, 7 or 8. In a further preferred embodiment, said stretch of consecutive negative amino acids has a length of 4 or 8 amino acids. In a further preferred embodiment, said stretch of consecutive negative amino acids has a length of 3 amino acids. In a further preferred embodiment, said stretch of consecutive negative amino acids has a length of 4 amino acids. In a further preferred embodiment, said stretch of consecutive negative amino acids has a length of 5 amino acids. In a further preferred embodiment, said stretch of consecutive negative amino acids has a length of 6 amino acids. In a further preferred embodiment, said stretch of consecutive negative amino acids has a length of 7 amino acids. In a further preferred embodiment, said stretch of consecutive negative amino acids has a length of 8 amino acids. In a further preferred embodiment, said stretch of consecutive negative amino acids has a length of 9 amino acids.

In a further preferred embodiment, said stretch of consecutive negative amino acids are independently selected from aspartic acid or glutamic acid, wherein said aspartic acid or said glutamic acid is independently in each occasion selected from its L-configuration or its D-configuration. In a further preferred embodiment, said stretch of consecutive negative amino acids comprises at least one aspartic acid in the L-configuration or in the D-configuration. In a further preferred embodiment, said stretch of consecutive negative amino acids comprises at least one aspartic acid in the L-configuration. In a further preferred embodiment, said stretch of consecutive negative amino acids comprises at least one aspartic acid in the D-configuration. In a further preferred embodiment, said stretch of consecutive negative amino acids comprises at least one glutamic acid in the L-configuration or the D-configuration. In a further preferred embodiment, said stretch of consecutive negative amino acids comprises at least one glutamic acid in the L-configuration. In a further preferred embodiment, said stretch of consecutive negative amino acids comprises at least one glutamic acid in the D-configuration.

In a further preferred embodiment, said stretch of consecutive negative amino acids comprises at least one aspartic acid in the L-configuration and at least one glutamic acid in the L-configuration. In a further preferred embodiment, said stretch of consecutive negative amino acids consists of aspartic acid and glutamic acid, all in the L-configuration. In a further preferred embodiment, said stretch of consecutive negative amino acids consists of aspartic acid or glutamic acid, all in the L-configuration.

In a further preferred embodiment, said stretch of consecutive negative amino acids comprises at least one aspartic acid or at least one glutamic acid. In a further preferred embodiment, said stretch of consecutive negative amino acids comprises at least two aspartic acid or at least two glutamic acid. In a further preferred embodiment, said stretch of consecutive negative amino acids comprises at least three aspartic acid or at least three glutamic acid. In a further preferred embodiment, said stretch of consecutive negative amino acids comprises at least four aspartic acid or at least four glutamic acid. In a further preferred embodiment, said stretch of consecutive negative amino acids comprises at least four aspartic acid. In a further preferred embodiment, said stretch of consecutive negative amino acids comprises at least four glutamic acid. In a further preferred embodiment, said stretch of consecutive negative amino acids comprises at least five glutamic acid. In a further preferred embodiment, said stretch of consecutive negative amino acids comprises at least six glutamic acid. In a further preferred embodiment, said stretch of consecutive negative amino acids comprises at least seven glutamic acid. In a further preferred embodiment, said stretch of consecutive negative amino acids comprises at least eight glutamic acid. In a further preferred embodiment, said stretch of consecutive negative amino acids consist solely of aspartic acid. In a further very preferred embodiment, said stretch of consecutive negative amino acids consists solely of glutamic acids.

In a further preferred embodiment, said stretch of consecutive negative amino acids comprises at least one aspartic acid or at least one glutamic acid, wherein said at least one aspartic acid or said at least one glutamic acid are in the L-configuration. In a further preferred embodiment, said stretch of consecutive negative amino acids comprises at least two aspartic acid or at least two glutamic acid, wherein at least two aspartic acid or at least two glutamic acid are in the L-configuration. In a further preferred embodiment, said stretch of consecutive negative amino acids comprises at least three aspartic acid or at least three glutamic acid, wherein said at least three aspartic acid or said at least three glutamic acid are in the L-configuration. In a further preferred embodiment, said stretch of consecutive negative amino acids comprises at least four aspartic acid or at least four glutamic acid, wherein said at least four aspartic acid or said at least four glutamic acid are in the L-configuration. In a further preferred embodiment, said stretch of consecutive negative amino acids comprises at least four aspartic acid, wherein said at least four aspartic acid are in the L-configuration. In a further preferred embodiment, said stretch of consecutive negative amino acids comprises at least four glutamic acid, wherein said at least four glutamic acid are in the L-configuration. In a further preferred embodiment, said stretch of consecutive negative amino acids comprises at least five glutamic acid, wherein said at least five glutamic acid are in the L-configuration. In a further preferred embodiment, said stretch of consecutive negative amino acids comprises at least six glutamic acid, wherein said at least six glutamic acid are in the L-configuration. In a further preferred embodiment, said stretch of consecutive negative amino acids comprises at least seven glutamic acid, wherein said at least seven glutamic acid are in the L-configuration. In a further preferred embodiment, said stretch of consecutive negative amino acids comprises at least eight glutamic acid, wherein said at least eight glutamic acid are in the L-configuration. In a further preferred embodiment, said stretch of consecutive negative amino acids consist solely of aspartic acids, wherein said aspartic acids are in the L-configuration. In a further very preferred embodiment, said stretch of consecutive negative amino acids consists solely of glutamic acids, wherein said glutamic acids are in the L-configuration.

In a preferred embodiment, said stretch of consecutive negative amino acids has a length of 3 to 10 amino acids, wherein said stretch of consecutive negative amino acids consists solely of glutamic acids. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids. In a further preferred embodiment, said stretch of consecutive negative amino acids has a length of 3 to 9 amino acids, wherein said stretch of consecutive negative amino acids consists solely of glutamic acids. In a further preferred embodiment, said stretch of consecutive negative amino acids has a length of 3 to 8 amino acids, wherein said stretch of consecutive negative amino acids consists solely of glutamic acids. In a further preferred embodiment, said stretch of consecutive negative amino acids has a length of 4 to 9 amino acids, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 or 8 amino acids, wherein said stretch of consecutive negative amino acids consists solely of glutamic acids. In a further preferred embodiment, said stretch of consecutive negative amino acids has a length of 3 amino acids, wherein said stretch of consecutive negative amino acids consists solely of glutamic acids. In a further preferred embodiment, said stretch of consecutive negative amino acids has a length of 4 amino acids, wherein said stretch of consecutive negative amino acids consists solely of glutamic acids. In a further preferred embodiment, said stretch of consecutive negative amino acids has a length of 5 amino acids, wherein said stretch of consecutive negative amino acids consists solely of glutamic acids. In a further preferred embodiment, said stretch of consecutive negative amino acids has a length of 6 amino acids, wherein said stretch of consecutive negative amino acids consists solely of glutamic acids. In a further preferred embodiment, said stretch of consecutive negative amino acids has a length of 7 amino acids, wherein said stretch of consecutive negative amino acids consists solely of glutamic acids. In a further preferred embodiment, said stretch of consecutive negative amino acids has a length of 8 amino acids, wherein said stretch of consecutive negative amino acids consists solely of glutamic acids. In a further preferred embodiment, said stretch of consecutive negative amino acids has a length of 9 amino acids, wherein said stretch of consecutive negative amino acids consists solely of glutamic acids.

In a preferred embodiment, said stretch of consecutive negative amino acids has a length of 3 to 10 amino acids, wherein said stretch of consecutive negative amino acids consists solely of glutamic acids, wherein said glutamic acids are in the L-configuration. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 or 8 amino acids, wherein said stretch of consecutive negative amino acids consists solely of glutamic acids, wherein said glutamic acids are in the L-configuration. In a further preferred embodiment, said stretch of consecutive negative amino acids has a length of 3 amino acids, wherein said stretch of consecutive negative amino acids consists solely of glutamic acids, wherein said glutamic acids are in the L-configuration. In a further preferred embodiment, said stretch of consecutive negative amino acids has a length of 4 amino acids, wherein said stretch of consecutive negative amino acids consists solely of glutamic acids, wherein said glutamic acids are in the L-configuration. In a further preferred embodiment, said stretch of consecutive negative amino acids has a length of 5 amino acids, wherein said stretch of consecutive negative amino acids consists solely of glutamic acids, wherein said glutamic acids are in the L-configuration. In a further preferred embodiment, said stretch of consecutive negative amino acids has a length of 6 amino acids, wherein said stretch of consecutive negative amino acids consists solely of glutamic acids, wherein said glutamic acids are in the L-configuration. In a further preferred embodiment, said stretch of consecutive negative amino acids has a length of 7 amino acids, wherein said stretch of consecutive negative amino acids consists solely of glutamic acids, wherein said glutamic acids are in the L-configuration. In a further preferred embodiment, said stretch of consecutive negative amino acids has a length of 8 amino acids, wherein said stretch of consecutive negative amino acids consists solely of glutamic acids, wherein said glutamic acids are in the L-configuration. In a further preferred embodiment, said stretch of consecutive negative amino acids has a length of 9 amino acids, wherein said stretch of consecutive negative amino acids consists solely of glutamic acids, wherein said glutamic acids are in the L-configuration.

In a further very preferred embodiment, said stretch of consecutive negative amino acids comprises SEQ ID NO:1 or SEQ ID NO:2. In a further very preferred embodiment, said stretch of consecutive negative amino acids consists of SEQ ID NO:1 or SEQ ID NO:2. In a further very preferred embodiment, said stretch of consecutive negative amino acids comprises SEQ ID NO:1. In a further very preferred embodiment, said stretch of consecutive negative amino acids consists of SEQ ID NO:1. In a further very preferred embodiment, said stretch of consecutive negative amino acids comprises SEQ ID NO:2. In a further very preferred embodiment, said stretch of consecutive negative amino acids consists of SEQ ID NO: 2.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids. In a preferred embodiment, said polypeptide further comprises a first amino acid linker, wherein said first amino acid linker is positioned at the N-terminus of said stretch of consecutive negative amino acids. In a preferred embodiment, said polypeptide comprising said stretch of consecutive negative amino acids further comprises a first amino acid linker, wherein said first amino acid linker is positioned at the C-terminus of said stretch of consecutive negative amino acids. In a preferred embodiment, said polypeptide comprising said stretch of consecutive negative amino acids further comprises a second amino acid linker. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids.

In a preferred embodiment, said first amino acid linker has a length of at most 30 amino acids. In a preferred embodiment, said first amino acid linker has a length of at most 20, 19, 18, 17 or 16 amino acids. In a preferred embodiment, said first amino acid linker has a length of at most 15 amino acids. In a preferred embodiment, said first amino acid linker has a length of at most 14 amino acids. In a preferred embodiment, said first amino acid linker has a length of at most 13 amino acids. In a preferred embodiment, said first amino acid linker has a length of at most 12 amino acids. In a preferred embodiment, said first amino acid linker has a length of at most 11 amino acids. In a preferred embodiment, said first amino acid linker has a length of at most 10 amino acids. In a preferred embodiment, said first amino acid linker has a length of at most 9 amino acids. In a preferred embodiment, said first amino acid linker has a length of at most 8 amino acids. In a preferred embodiment, said first amino acid linker has a length of at most 7 amino acids. In a preferred embodiment, said first amino acid linker has a length of at most 6 amino acids. In a preferred embodiment, said first amino acid linker has a length of at most 5 amino acids. In a preferred embodiment, said first amino acid linker has a length of at most 4 amino acids. In a preferred embodiment, said first amino acid linker has a length of at most 3 amino acids. In a preferred embodiment, said first amino acid linker has a length of at most 2 amino acids. In a preferred embodiment, said first amino acid linker consists of one amino acid. In a preferred embodiment, said second amino acid linker has a length of at most 30 amino acids. In a preferred embodiment, said second amino acid linker has a length of at most 20, 19, 18, 17 or 16 amino acids. In a preferred embodiment, said second amino acid linker has a length of at most 15 amino acids. In a preferred embodiment, said second amino acid linker has a length of at most 14 amino acids. In a preferred embodiment, said second amino acid linker has a length of at most 13 amino acids. In a preferred embodiment, said second amino acid linker has a length of at most 12 amino acids. In a preferred embodiment, said second amino acid linker has a length of at most 11 amino acids. In a preferred embodiment, said second amino acid linker has a length of at most 10 amino acids. In a preferred embodiment, said second amino acid linker has a length of at most 9 amino acids. In a preferred embodiment, said second amino acid linker has a length of at most 8 amino acids. In a preferred embodiment, said second amino acid linker has a length of at most 7 amino acids. In a preferred embodiment, said second amino acid linker has a length of at most 6 amino acids. In a preferred embodiment, said second amino acid linker has a length of at most 5 amino acids. In a preferred embodiment, said second amino acid linker has a length of at most 4 amino acids. In a preferred embodiment, said second amino acid linker has a length of at most 3 amino acids. In a preferred embodiment, said second amino acid linker has a length of at most 2 amino acids. In a preferred embodiment, said second amino acid linker consists of one amino acid.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids, and wherein said first amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a second amino acid linker, wherein said second amino acid linker is positioned at the N- or at the C-terminus of said stretch of consecutive negative amino acids, and wherein said second amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1; and an amino acid linker (GS*-linker) comprising at least one Gly, at least one Ser, and at least one amino acid selected from Thr, Ala, Lys, and Cys.

In a preferred embodiment, said first amino acid linker is a polyglycine linker (Gly)ₙ of a length of n=2-10. In a preferred embodiment, said first amino acid linker is a glycine-serine linker (GS-linker) comprising at least one glycine and at least one serine. In a preferred embodiment, said first amino acid linker is a glycine-serine linker (GS-linker) comprising at least one glycine and at least one serine, and wherein said first amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, and u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said first amino acid linker is a glycine-serine linker (GS-linker), and said GS linker has an amino acid sequence of SEQ ID NO:8. In a further preferred embodiment, said first amino acid linker has an amino acid sequence of SEQ ID NO: 8. In a preferred embodiment, said first amino acid linker is an amino acid linker (GS*-linker) comprising at least one Gly, at least one Ser, and at least one amino acid selected from Thr, Ala, Lys, and Cys.

In a preferred embodiment, said second amino acid linker is a polyglycine linker (Gly)ₙ of a length of n=2-10. In a preferred embodiment, said second amino acid linker is a glycine-serine linker (GS-linker) consisting of at least one glycine and at least one serine. In a preferred embodiment, said second amino acid linker is a glycine-serine linker (GS-linker) comprising at least one glycine and at least one serine, and wherein said second amino acid linker has a Gly-Ser at its N-terminus. In a further preferred embodiment, said second amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said second amino acid linker is a glycine-serine linker (GS-linker), and said GS linker has the amino acid sequence of SEQ ID NO:9.

In a preferred embodiment, said second amino acid linker is an amino acid linker selected from Thr, Ala, Lys, and Cys. In a preferred embodiment, said second amino acid linker is an amino acid linker (GS*-linker) comprising at least one Gly, at least one Ser, and at least Cys. In a preferred embodiment, said second amino acid linker is an amino acid linker comprising at least one Gly, at least one Ser, and at least Cys (GS*-linker), and said second amino acid linker has a Gly-Ser at its N-terminus. In a further preferred embodiment, said second amino acid linker (GS*-linker) has a length of at most 15, 14, 13, 12, 11, preferably 10, 9, and further preferably a length of at most 7 or 6 amino acids. In a further preferred embodiment, said second amino acid linker is amino acid linker (GS*-linker), and said GS*-linker has the amino acid sequence of SEQ ID NO:4.

In a preferred embodiment, said first and said second amino acid linker are independently a polyglycine linker (Gly)ₙ of a length of n=2-10. In a preferred embodiment, said first and said second amino acid linker are independently a glycine-serine linker (GS-linker) comprising at least one glycine and at least one serine. In a preferred embodiment, said first and said second amino acid linker are independently an amino acid linker (GS*-linker) comprising at least one Gly, at least one Ser, and at least one amino acid selected from Thr, Ala, Lys, and Cys, and wherein said second amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first and said second amino acid linker is independently a glycine-serine linker (GS-linker), said GS linker has an amino acid sequence of (GS)ᵣ(GₛS)ₜ(GS)ᵤ with r=0 or 1, s=2, 3 or 4, t=1, 2 or 3, u=0 or 1.

In a further preferred embodiment, said first amino acid linker and/or said second amino linker comprises, preferably consists of, of an amino acid sequence selected from SEQ ID NO:4, SEQ ID NO:8 and SEQ ID NO:9. In a further very preferred embodiment, said first amino acid linker comprises, preferably consists of, SEQ ID NO:8. In a further very preferred embodiment, said second amino acid linker comprises, preferably consists of, SEQ ID NO:4 or SEQ ID NO:9. In a further very preferred embodiment, said second amino acid linker comprises, preferably consists of, SEQ ID NO:4. In a further very preferred embodiment, said second amino acid linker comprises, preferably consists of, SEQ ID NO:9. In a further very preferred embodiment, said first amino acid linker comprises, preferably consists of, SEQ ID NO:8 and said second amino acid linker comprises, preferably consists of, SEQ ID NO:4 or SEQ ID NO:9. In a further very preferred embodiment, said first amino acid linker comprises, preferably consists of, SEQ ID NO:8 and said second amino acid linker comprises, preferably consists of, SEQ ID NO:4. In a further very preferred embodiment, said first amino acid linker comprises, preferably consists of, SEQ ID NO:8 and said second amino acid linker comprises, preferably consists of, or SEQ ID NO:9.

In a preferred embodiment, said polypeptide comprising, preferably consisting of, a stretch of consecutive negative amino acids has a length of at most 30 amino acids. In a preferred embodiment, said polypeptide has a length of at most 25, 24, 23, 22, or 21 amino acids. In a preferred embodiment, said polypeptide has a length of at most 20 amino acids. In a preferred embodiment, said polypeptide has a length of at most 19 amino acids. In a preferred embodiment, said polypeptide has a length of at most 18 amino acids. In a preferred embodiment, said polypeptide has a length of at most 17 amino acids. In a preferred embodiment, said polypeptide has a length of at most 16 amino acids. In a preferred embodiment, said polypeptide has a length of at most 15 amino acids. In a preferred embodiment, said polypeptide has a length of at most 14 amino acids. In a preferred embodiment, said polypeptide has a length of at most 13 amino acids. In a preferred embodiment, said polypeptide has a length of at most 12 amino acids. In a preferred embodiment, said polypeptide has a length of at most 11 amino acids. In a preferred embodiment, said polypeptide has a length of at most 10 amino acids. In a preferred embodiment, said polypeptide has a length of at most 9 amino acids. In a preferred embodiment, said polypeptide has a length of at most 8 amino acids. In a preferred embodiment, said polypeptide has a length of at most 7 amino acids. In a preferred embodiment, said polypeptide has a length of at most 6 amino acids. In a preferred embodiment, said polypeptide has a length of at most 5 amino acids. In a preferred embodiment, said polypeptide has a length of at most 4 amino acids. In a further preferred embodiment, said polypeptide consists of said stretch of consecutive negative amino acids.

In a further very preferred embodiment, said polypeptide comprises SEQ ID NO:49, SEQ ID NO:50 or SEQ ID NO:51. In a further very preferred embodiment, said polypeptide consists of SEQ ID NO:49, SEQ ID NO:50 or SEQ ID NO:51. In a further very preferred embodiment, said polypeptide comprises SEQ ID NO:49. In a further very preferred embodiment, said polypeptide comprises SEQ ID NO:50. In a further very preferred embodiment, said polypeptide comprises SEQ ID NO:51. In a further very preferred embodiment, said polypeptide consists of SEQ ID NO:49. In a further very preferred embodiment, said polypeptide consists of SEQ ID NO:50. In a further very preferred embodiment, said polypeptide consists of SEQ ID NO:51.

In a further preferred embodiment, said polypeptide comprising, preferably consisting of, a stretch of consecutive negative amino acids is inserted between any amino acid residue of said CMV polypeptide corresponding to any amino acid residue between position 75 and position 85 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 75 and position 76 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 76 and position 77 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 77 and position 78 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 78 and position 79 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 79 and position 80 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 80 and position 81 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 75 and position 81 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 82 and position 83 of SEQ ID NO:39. In a further preferred embodiment, said polypeptide comprising, preferably consisting of, a stretch of consecutive negative amino acids is inserted between amino acid residues of said CMV polypeptide corresponding to amino acid residues of position 83 and position 84 of SEQ ID NO:39. In a further very preferred embodiment, said polypeptide comprising, preferably consisting of, a stretch of consecutive negative amino acids is inserted between amino acid residues of said CMV polypeptide corresponding to amino acid residues of position 84 and position 85 of SEQ ID NO:39.

In a very preferred embodiment, said CMV polypeptide comprises the amino acid sequence of SEQ ID NO:5, SEQ ID NO:39 or SEQ ID NO:48, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:39, or between amino acid residues of position 86 (Ser) and position 87 (Thr) of SEQ ID NO:48. In a very preferred embodiment, said CMV polypeptide consists of the amino acid sequence of SEQ ID NO:5, SEQ ID NO:39 or SEQ ID NO:48, wherein said polypeptide comprising said stretch of consecutive negative amino acids is inserted between amino acid residues of position 88 and position 89 of said SEQ ID NO:5, between amino acid residues of position 84 and position 85 of SEQ ID NO:39, or between amino acid residues of position 86 and position 87 of SEQ ID NO: 48. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:39, wherein said polypeptide is inserted between amino acid residues of position 84 and position 85 of SEQ ID NO: 39. In a very preferred embodiment, said CMV polypeptide comprises the amino acid sequence of SEQ ID NO:48, wherein said polypeptide is inserted between amino acid residues of position 86 and position 87 of SEQ ID NO:48. In a very preferred embodiment, said CMV polypeptide consists of the amino acid sequence of SEQ ID NO:5, wherein said polypeptide is inserted between amino acid residues of position 88 and position 89 of SEQ ID NO: 5. In a very preferred embodiment, said CMV polypeptide consists of the amino acid sequence of SEQ ID NO:39, wherein said polypeptide is inserted between amino acid residues of position 84 and position 85 of SEQ ID NO:39. In a very preferred embodiment, said CMV polypeptide consists of the amino acid sequence of SEQ ID NO:48, wherein said polypeptide is inserted between amino acid residues of position 86 and position 87 of SEQ ID NO: 48.

In a very preferred embodiment, said chimeric CMV polypeptide comprises the amino acid sequence of SEQ ID NO:5, SEQ ID NO:39 or SEQ ID NO:48, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:39, or between amino acid residues of position 86 (Ser) and position 87 (Thr) of SEQ ID NO:48. In a very preferred embodiment, said chimeric CMV polypeptide consists of the amino acid sequence of SEQ ID NO: 5, SEQ ID NO:39 or SEQ ID NO:48, wherein said polypeptide comprising said stretch of consecutive negative amino acids is inserted between amino acid residues of position 88 and position 89 of said SEQ ID NO:5, between amino acid residues of position 84 and position 85 of SEQ ID NO:39, or between amino acid residues of position 86 and position 87 of SEQ ID NO:48. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:39, wherein said polypeptide is inserted between amino acid residues of position 84 and position 85 of SEQ ID NO:39. In a very preferred embodiment, said chimeric CMV polypeptide comprises the amino acid sequence of SEQ ID NO:48, wherein said polypeptide is inserted between amino acid residues of position 86 and position 87 of SEQ ID NO:48. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:39, wherein said polypeptide is inserted between amino acid residues of position 84 and position 85 of SEQ ID NO:39. In a very preferred embodiment, said chimeric CMV polypeptide consists of the amino acid sequence of SEQ ID NO:48, wherein said polypeptide is inserted between amino acid residues of position 86 and position 87 of SEQ ID NO: 48.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO: 12. In a very preferred embodiment, said chimeric CMV polypeptide comprises the amino acid sequence of SEQ ID NO: 10. In a very preferred embodiment, said chimeric CMV polypeptide comprises the amino acid sequence of SEQ ID NO: 11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO: 10, SEQ ID NO:11 or SEQ ID NO:12. In a very preferred embodiment, said chimeric CMV polypeptide consists of the amino acid sequence of SEQ ID NO: 10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO: 12.

Thus, in another aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO: 11 or SEQ ID NO:12;
(b) at least one antigen, wherein said antigen comprises at least one second attachment site, and wherein said antigen is a nerve growth factor (NGF) antigen; and
wherein (a) and (b) are linked through said at least one first and said at least one second disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain.

In embodiment, said chimeric CMV polypeptide comprises the amino acid sequence of SEQ ID NO:10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO:12. In a very preferred embodiment, said modified VLP of CMV comprises 180 identical chimeric CMV polypeptides, wherein said chimeric CMV polypeptide comprises, preferably consists of, the amino acid sequence of SEQ ID NO:10, SEQ ID NO:11 or SEQ ID NO:12. In a very preferred embodiment, said modified VLP of CMV comprises 180 identical chimeric CMV polypeptides, wherein said chimeric CMV polypeptide comprises, preferably consists of, the amino acid sequence of SEQ ID NO:10. In a very preferred embodiment, said modified VLP of CMV comprises 180 identical chimeric CMV polypeptides, wherein said chimeric CMV polypeptide comprises, preferably consists of, the amino acid sequence of SEQ ID NO:11. In a very preferred embodiment, said modified VLP of CMV comprises 180 identical chimeric CMV polypeptides, wherein said chimeric CMV polypeptide comprises, preferably consists of, the amino acid sequence of SEQ ID NO:12.

The modified CMV VLPs of the invention may be expressed in prokaryotic or eukaryotic expression systems. Preferred systems are E. coli, yeast, insect cells as well as mammalian cell lines. Very preferred said modified VLP of CMV is obtained by expression of said chimeric CMV polypeptide in E. coli., and wherein preferably said expression is effected at temperatures of between 10° C. to 25° C., preferably at a temperature of 20° C. As indicated above, recombinantly produced polypeptides may comprise an N-terminal methionine residue. In one embodiment said chimeric CMV polypeptide therefore comprises an N-terminal methionine residue. However, typically and preferably said N-terminal methionine residue is cleaved off said chimeric CMV polypeptide.

In a further preferred embodiment said modified VLP of CMV further comprises at least one immunostimulatory substance. In a very preferred embodiment, said immunostimulatory substance is packaged into the modified VLPs of the invention. In another preferred embodiment, the immunostimulatory substance is mixed with the modified VLPs of the invention. Immunostimulatory substances useful for the invention are generally known in the art and are disclosed, inter alia, in WO2003/024481.

In another embodiment of the present invention, said immunostimulatory substance consists of DNA or RNA of non-eukaryotic origin. In a further preferred embodiment said immunostimulatory substance is selected from the group consisting of: (a) immunostimulatory nucleic acid; (b) peptidoglycan; (c) lipopolysaccharide; (d) lipoteichonic acid; I imidazoquinoline compound; (f) flagelline; (g) lipoprotein; and (h) any mixtures of at least one substance of (a) to (g). In a further preferred embodiment said immunostimulatory substance is an immunostimulatory nucleic acid, wherein said immunostimulatory nucleic acid is selected from the group consisting of: (a) ribonucleic acids; (b) deoxyribonucleic acids; (c) chimeric nucleic acids; and (d) any mixture of (a), (b) and/or (c). In a further preferred embodiment said immunostimulatory nucleic acid is a ribonucleic acid, and wherein said ribonucleic acid is bacteria derived RNA. In a further preferred embodiment said immunostimulatory nucleic acid is poly (IC) or a derivative thereof. In a further preferred embodiment said immunostimulatory nucleic acid is a deoxyribonucleic acid, wherein said deoxyribonucleic acid is an unmethylated CpG-containing oligonucleotide.

In a very preferred embodiment said immunostimulatory substance is an unmethylated CpG-containing oligonucleotide. In a further preferred embodiment said unmethylated CpG-containing oligonucleotide is an A-type CpG. In a further preferred embodiment said A-type CpG comprises a palindromic sequence. In a further preferred embodiment said palindromic sequence is flanked at it ‘5’-terminus and at its ‘3’-terminus by guanosine entities. In a further preferred embodiment said palindromic sequence is flanked at its ‘5’-terminus by at least 3 and at most 15 guanosine entities, and wherein said palindromic sequence is flanked at its ‘3’-terminus by at least 3 and at most 15 guanosine entities.

In another preferred embodiment, said immunostimulatory substance is an unmethylated CpG-containing oligonucleotide, and wherein preferably said unmethylated CpG-containing oligonucleotide comprises a palindromic sequence, and wherein further preferably the CpG motif of said unmethylated CpG-containing oligonucleotide is part of a palindromic sequence, and wherein further preferably said palindromic sequence is SEQ ID NO: 52. In a further preferred embodiment, said immunostimulatory nucleic acid is an unmethylated CpG containing oligonucleotide consisting of SEQ ID NO:53, wherein said unmethylated CpG-containing oligonucleotide consists exclusively of phosphodiester bound nucleotides.

In a further aspect, the present invention provides a composition comprising (a) modified VLP of CMV as defined herein, wherein said modified VLP of CMV comprises at least one first attachment site; and (b) at least one NGF antigen, wherein said antigen comprises at least one second attachment site; wherein (a) and (b) are linked through said at least one first and said at least one second attachment site, typically and preferably via at least one covalent non-peptide bond. Methods for linking said modified VLP and said antigens via said first and said second attachment site are described, for example, in WO2002/056905, WO2004/084940 and WO2016/062720.

Thus, in a further aspect, the present invention provides for a composition for use in a method of treating a NGF-related disorder, in canine comprising the administration of said composition to said canine, wherein the composition comprises
(a) modified VLP of CMV, wherein said modified VLP of CMV comprises at least one first attachment site; and
(b) at least one NGF antigen, wherein said NGF antigen comprises at least one second attachment site;
(c) optionally or even preferably a T helper cell epitope,
wherein (a) and (b) are linked through said at least one first and said at least one second attachment site, typically and preferably via at least one covalent non-peptide bond. Preferably said modified VLP of CMV comprises at least one chimeric CMV polypeptide, wherein said at least one chimeric CMV polypeptide comprises, preferably consists of, (i) a CMV polypeptide, wherein said CMV polypeptide comprises a coat protein of CMV or an amino acid sequence having a sequence identity of at least 75% with SEQ ID NO:39; and (ii) preferably a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:39.

In a very preferred embodiment, said at least one first attachment site is not comprised or is not part of the polypeptide comprising said stretch of consecutive negative amino acids. In a very preferred embodiment, all of said first attachments sites are not comprised or are not part of the polypeptide comprising said stretch of consecutive negative amino acids. In a very preferred embodiment, said at least one first attachment site is not comprised or is not part of the stretch of consecutive negative amino acids. In a very preferred embodiment, all of said first attachments sites are not comprised or are not part of the stretch of consecutive negative amino acids. In a very preferred embodiment, said first attachment site and said second attachment site are linked solely via one or more covalent bonds. In a very preferred embodiment, said at least one antigen is linked to said modified VLP of CMV solely via one or more covalent bonds. In a very preferred embodiment, all of said antigens are linked to said modified VLP of CMV solely via one or more covalent bonds.

In a further preferred embodiment, said first attachment site is linked to said second attachment site via at least one covalent non-peptide bond. In a further preferred embodiment, all of said first attachment sites are linked to said second attachment sites via at least one covalent non-peptide bond. In a further very preferred embodiment, said first attachment site is an amino group, preferably an amino group of a lysine. In a further very preferred embodiment, all of said first attachment sites are an amino group, preferably an amino group of a lysine.

Thus, in a further aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO:11 or SEQ ID NO:12;
(b) at least one antigen, wherein said antigen comprises at least one second attachment site, and wherein said antigen is a nerve growth factor (NGF) antigen; and
wherein (a) and (b) are linked through said at least one first and said at least one second attachment site via at least one covalent non-peptide bond; and wherein said at least one first attachment site is not comprised or is not part of the polypeptide comprising said stretch of consecutive negative amino acids; and wherein preferably said first attachment sites are an amino group, hereby preferably an amino group of a lysine, and wherein further preferably the second attachment sites are a sulfhydryl group, preferably a sulfhydryl group of a cysteine residue or a sulfhydryl group that has been chemically attached to the NGF antigen. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. Preferably said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. Preferably, said (NGF-containing CMV VLP) composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

Attachment between modified virus-like particles and antigens by way of disulfide bonds are typically labile, in particular, to sulfhydryl-moiety containing molecules, and are, furthermore, less stable in serum than, for example, thioether attachments (Martin F J. and Papahadjopoulos D. (1982) J. Biol. Chem. 257:286-288). Therefore, in a further very preferred embodiment of the present invention, the association or linkage of the modified VLP of CMV and the at least one antigen does not comprise a disulfide bond. Further preferred hereby, the at least one second attachment site comprise, or preferably is, a sulfhydryl group. Preferably, all of said second attachment sites comprise, or preferably are, a sulfhydryl group. In a further preferred embodiment, said at least one first attachment site is not or does not comprise a sulfhydryl group. In a further preferred embodiment, all of said first attachment sites are not or do not comprise a sulfhydryl group. In a preferred embodiment, said at least one first attachment site is not or does not comprise a sulfhydryl group of a cysteine. In a preferred embodiment, all of said first attachment sites are not or do not comprise a sulfhydryl group of a cysteine. In a further very preferred embodiment said second attachment site is a sulfhydryl group, preferably a sulfhydryl group of a cysteine. In a further very preferred embodiment, all of said second attachment sites are a sulfhydryl group, preferably a sulfhydryl group of a cysteine.

In a very preferred embodiment, the at least one first attachment site is an amino group, preferably an amino group of a lysine residue and the at least one second attachment site is a sulfhydryl group, preferably a sulfhydryl group of a cysteine residue or a sulfhydryl group that has been chemically attached to the antigen. In a very preferred embodiment, all of said first attachment sites are an amino group, preferably an amino group of a lysine residue and all of said second attachment sites are a sulfhydryl group, preferably a sulfhydryl group of a cysteine residue or a sulfhydryl group that has been chemically attached to the antigen. In a further preferred embodiment only one of said second attachment sites associates with said first attachment site through at least one non-peptide covalent bond leading to a single and uniform type of binding of said antigen to said modified VLP of CMV, wherein said only one second attachment site that associates with said first attachment site is a sulfhydryl group, and wherein said antigen and said modified VLP of CMV interact through said association to form an ordered and repetitive antigen array.

In one preferred embodiment of the invention, the antigen is linked to the modified VLP of CMV by way of chemical cross-linking, typically and preferably by using a heterobifunctional cross-linker.

Thus, in a preferred embodiment, the NGF antigen is linked to the modified VLP of CMV by way of chemical cross-linking, typically and preferably by way of a heterobifunctional cross-linker through said at least one first and said at least one second attachment site via at least one covalent non-peptide bond. In preferred embodiments, the hetero-bifunctional cross-linker contains a functional group which can react with the preferred first attachment sites, preferably with the amino group, more preferably with the amino groups of lysine residue(s) of the modified VLP of CMV, and a further functional group which can react with the preferred second attachment site, i.e. a sulfhydryl group, preferably of cysteine(s) residue inherent of, or artificially added to the antigen, and optionally also made available for reaction by reduction. Several hetero-bifunctional cross-linkers are known to the art. These include the preferred cross-linkers succinimidyl-6-(b-maleimidopropionamide) hexanoate (SMPH) (Pierce), Sulfo-MBS, Sulfo-EMCS, Sulfo-GMBS, Sulfo-SIAB, Sulfo-SMPB, Sulfo-SMCC, Sulfo-KMUS SVSB, SIA, and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. In a very preferred embodiment, said hetero-bifunctional cross-linker is SMPH. Thus, in a preferred embodiment, the NGF antigen is linked to the modified VLP of CMV by way of chemical cross-linking, typically and preferably by way of a heterobifunctional cross-linker through said at least one first and said at least one second attachment site via at least one covalent non-peptide bond, and wherein said hetero-bifunctional cross-linker is SMPH. Another class of cross-linkers suitable in the practice of the invention is characterized by the introduction of a disulfide linkage between the antigen and the modified VLP upon coupling. Preferred cross-linkers belonging to this class include, for example, SPDP and Sulfo-LC-SPDP (Pierce).

Thus, in a further aspect, the present invention provides for a composition for use in a method of treating a NGF-related disorder, in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10, SEQ ID NO:11 or SEQ ID NO:12;
(b) at least one antigen, wherein said antigen comprises at least one second attachment site, and wherein said antigen is a nerve growth factor (NGF) antigen; and
wherein (a) and (b) are linked by way of a heterobifunctional cross-linker through said at least one first and said at least one second attachment site via at least one covalent non-peptide bond, preferably wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group of a cysteine residue or a sulfhydryl group that has been chemically attached to the NGF antigen. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. Preferably said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. Preferably, said (NGF-containing CMV VLP) composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO: 12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

Linking of the antigen to the modified VLP of CMV by using a hetero-bifunctional cross-linker allows linking of the antigen to the modified VLP of CMV in an oriented fashion. Other methods of linking the antigen to the modified VLP of CMV include methods wherein the antigen is cross-linked to the modified VLP of CMV, using the carbodiimide EDC, and NHS. The antigen may also be first thiolated through reaction, for example with SATA, SATP or iminothiolane. The antigen, after deprotection if required, may then be coupled to the modified VLP of CMV as follows. After separation of the excess thiolation reagent, the antigen is reacted with the modified VLP of CMV, previously activated with a hetero-bifunctional cross-linker comprising a cysteine reactive moiety, and therefore displaying at least one or several functional groups reactive towards cysteine residues, to which the thiolated antigen can react, such as described above. Optionally, low amounts of a reducing agent are included in the reaction mixture. In further methods, the antigen is attached to the modified VLP of CMV, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the modified VLP.

In very preferred embodiments of the invention, the antigen is linked via a cysteine residue, having been added to either the N-terminus or the C-terminus of, or a natural cysteine residue within the antigen, to lysine residues of the modified VLP of CMV. In a preferred embodiment, the composition of the invention further comprises a linker, wherein said linker associates said antigen with said second attachment site, and wherein preferably said linker comprises or alternatively consists of said second attachment site.

Engineering of a second attachment site onto the antigen is achieved by the association of a linker, typically and preferably containing at least one amino acid suitable as second attachment site according to the disclosures of this invention. Therefore, in a preferred embodiment of the present invention, a linker is associated to the antigen by way of at least one covalent bond, preferably, by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the linker comprises or is a cysteine residue. In a further preferred embodiment of the present invention, the linker consists of amino acids, wherein further preferably the linker consists at most 15 amino acids. In an preferred embodiment of the invention, such amino acid linker contains 1 to 10 amino acids.

In a further aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10;
(b) at least one antigen, wherein said antigen comprises at least one second attachment site, and wherein said antigen is a nerve growth factor (NGF) antigen; and
wherein (a) and (b) are linked by way of a heterobifunctional cross-linker through said at least one first and said at least one second attachment site via at least one covalent non-peptide bond, wherein said heterobifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group that has been chemically attached to the NGF antigen. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. Preferably said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. Preferably, said (NGF-containing CMV VLP) composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

In a very preferred embodiment, said chimeric CMV polypeptide consists of the amino acid sequence of SEQ ID NO:10. In a very preferred embodiment, said modified VLP of CMV comprises 180 identical chimeric CMV polypeptides, wherein said chimeric CMV polypeptide comprises, preferably consists of, the amino acid sequence of SEQ ID NO:10. In a very preferred embodiment, said modified VLP of CMV comprises 180 identical chimeric CMV polypeptides, wherein said chimeric CMV polypeptide consists of the amino acid sequence of SEQ ID NO:10.

In another preferred embodiment, said antigen is nerve growth factor (NGF) selected from human NGF (hNGF), canine NGF (cNGF), feline NGF (fNGF), equine NGF (eNGF), bovine NGF (bNGF) and porcine NGF (pNGF), preferably canine NGF (cNGF) or feline NGF (fNGF), and wherein further preferably said antigen is canine NGF (cNGF). In a preferred embodiment, said antigen comprises, or preferably consists of, of an amino acid sequence selected from any of SEQ ID NO:30, SEQ ID NO:31, SEQ ID NO:33, SEQ ID NO: 54, SEQ ID NO:55, SEQ ID NO:56, SEQ ID NO:57, and SEQ ID NO:58, or an amino acid sequence having a sequence identity of at least 90% or at least 91%, preferably of at least 92%, at least 93% or at least 94%, further preferably of at least 95%, at least 96% or at least 97%, further preferably of at least 98% or at least 99% with any of SEQ ID NO:30, SEQ ID NO:31, SEQ ID NO:33, SEQ ID NO:54, SEQ ID NO:55, SEQ ID NO:56, SEQ ID NO: 57, and SEQ ID NO:58. In a further embodiment, said NGF antigen comprises a polyhistidine-tag of at least two consecutive and at most 12 consecutive histidine residues, preferably C-terminally or N-terminally positioned of the NGF antigen. In a further embodiment, said NGF antigen comprises a polyhistidine-tag of at least 2, 3, 4, 5, 6, 7, 8, 9, 10, 11 or 12 consecutive histidine residues, preferably C-terminally or N-terminally positioned of the NGF antigen. In a further embodiment, said NGF antigen comprises a polyhistidine-tag of 4, 6, 8 or 10 consecutive histidine residues, preferably C-terminally or N-terminally positioned of the NGF antigen. In a further embodiment, said NGF antigen comprises a polyhistidine-tag of 4 consecutive histidine residues, preferably C-terminally positioned of the NGF antigen. In a further embodiment, said NGF antigen comprises a polyhistidine-tag of 4 consecutive histidine residues, preferably N-terminally positioned of the NGF antigen. In a further embodiment, said NGF antigen comprises a polyhistidine-tag of 6 consecutive histidine residues consisting of SEQ ID NO:34, preferably C-terminally positioned of the NGF antigen. In a further embodiment, said NGF antigen comprises a polyhistidine-tag of 6 consecutive histidine residues consisting of SEQ ID NO:34, preferably N-terminally positioned of the NGF antigen. In a further embodiment, said NGF antigen comprises a polyhistidine-tag of 8 consecutive histidine residues, preferably C-terminally positioned of the NGF antigen. In a further embodiment, said NGF antigen comprises a polyhistidine-tag of 8 consecutive histidine residues, preferably N-terminally positioned of the NGF antigen. In a further embodiment, said NGF antigen comprises a polyhistidine-tag of 10 consecutive histidine residues, preferably C-terminally or N-terminally positioned of the NGF antigen.

In an alternative embodiment, said antigen is human NGF. In a further embodiment, said antigen comprises, or preferably consists of, SEQ ID NO:54 or an amino acid sequence having a sequence identity of at least 90%, preferably of at least 92%, further preferably of at least 95%, and further preferably of at least 98% amino acid sequence identity with SEQ ID NO: 54. In a further embodiment, said antigen comprises SEQ ID NO:54. In a further, said antigen consists of SEQ ID NO:54.

In a further very preferred embodiment, said antigen is canine NGF. In a very preferred embodiment, said antigen comprises, or preferably consists of, SEQ ID NO:30, SEQ ID NO: 31, SEQ ID NO:33 or an amino acid sequence having a sequence identity of at least 90%, preferably of at least 92%, further preferably of at least 95%, and further preferably of at least 98% amino acid sequence identity with SEQ ID NO:30 or SEQ ID NO:31 or SED ID NO: 33. In a further preferred embodiment, said antigen comprises SEQ ID NO:30 or SEQ ID NO:31 or SEQ ID NO:33. In a further preferred embodiment, said antigen consists of SEQ ID NO:30 or SEQ ID NO:31 or SEQ ID NO:33. In a further very preferred embodiment, said antigen comprises, or preferably consists of, SEQ ID NO:30 or an amino acid sequence having a sequence identity of at least 90%, preferably of at least 92%, further preferably of at least 95%, and further preferably of at least 98% amino acid sequence identity with SEQ ID NO:30. In a further very preferred embodiment, said antigen comprises SEQ ID NO:30. In a further very preferred embodiment, said antigen consists of SEQ ID NO: 30. In a further preferred embodiment, said antigen comprises, or preferably consists of, SEQ ID NO:31 or an amino acid sequence having a sequence identity of at least 90%, preferably of at least 92%, further preferably of at least 95%, and further preferably of at least 98% amino acid sequence identity with SEQ ID NO:31. In a further very preferred embodiment, said antigen comprises SEQ ID NO:31. In a further very preferred embodiment, said antigen consists of SEQ ID NO:31. In a further preferred embodiment, said antigen comprises, or preferably consists of, SEQ ID NO:33 or an amino acid sequence having a sequence identity of at least 90%, preferably of at least 92%, further preferably of at least 95%, and further preferably of at least 98% amino acid sequence identity with SEQ ID NO: 33. In a further very preferred embodiment, said antigen comprises SEQ ID NO:33. In a further very preferred embodiment, said antigen consists of SEQ ID NO:33.

In a further embodiment, said antigen comprises, or preferably consists of, SEQ ID NO: 30 or an amino acid sequence having a sequence identity of at least 90%, preferably of at least 92%, further preferably of at least 95%, and further preferably of at least 98% amino acid sequence identity with SEQ ID NO:30, and wherein said NGF antigen further comprises a polyhistidine-tag of at least two consecutive and at most 12 consecutive histidine residues, preferably 4, 6, 8, or 10 consecutive histidine residues, further preferably 6 consecutive histidine residues consisting of SEQ ID NO:34, and hereby preferably C-terminally or N-terminally positioned of the NGF antigen, further preferably C-terminally positioned of the NGF antigen. In a further embodiment, said antigen comprises, or preferably consists of, SEQ ID NO:30 or an amino acid sequence having a sequence identity of at least 90%, preferably of at least 92%, further preferably of at least 95%, and further preferably of at least 98% amino acid sequence identity with SEQ ID NO:30, and wherein said NGF antigen further comprises a polyhistidine-tag of at 4, 6, 8, or 10 consecutive histidine residues, preferably 6 consecutive histidine residues consisting of SEQ ID NO:34, and hereby preferably C-terminally or N-terminally positioned of the NGF antigen, further preferably C-terminally positioned of the NGF antigen. In a further embodiment, said antigen comprises, or preferably consists of, SEQ ID NO:30 or an amino acid sequence having a sequence identity of at least 90%, preferably of at least 92%, further preferably of at least 95%, and further preferably of at least 98% amino acid sequence identity with SEQ ID NO: 30, and wherein said NGF antigen further comprises a polyhistidine-tag of 6 consecutive histidine residues consisting of SEQ ID NO:34, and preferably C-terminally or N-terminally positioned of the NGF antigen, further preferably C-terminally positioned of the NGF antigen. In a further very preferred embodiment, said antigen comprises, or preferably consists of, SEQ ID NO:30 or an amino acid sequence having a sequence identity of at least 90%, preferably of at least 92%, further preferably of at least 95%, and further preferably of at least 98% amino acid sequence identity with SEQ ID NO:30, and wherein said NGF antigen further comprises a polyhistidine-tag of 6 consecutive histidine residues consisting of SEQ ID NO:34 and N-terminally positioned of the NGF antigen. In a further embodiment, said antigen comprises SEQ ID NO:30, and wherein said NGF antigen further comprises a polyhistidine-tag of at least two consecutive and at most 12 consecutive histidine residues, preferably 4, 6, 8, or 10 consecutive histidine residues, further preferably 6 consecutive histidine residues consisting of SEQ ID NO:34, and hereby preferably C-terminally or N-terminally positioned of the NGF antigen, further preferably C-terminally positioned of the NGF antigen. In a further embodiment, said antigen comprises SEQ ID NO:30, and wherein said NGF antigen further comprises a polyhistidine-tag of 6 consecutive histidine residues consisting of SEQ ID NO:34, and preferably C-terminally or N-terminally positioned of the NGF antigen, further preferably C-terminally positioned of the NGF antigen. In a further embodiment, said antigen comprises SEQ ID NO:30, and wherein said NGF antigen further comprises a polyhistidine-tag of 6 consecutive histidine residues consisting of SEQ ID NO: 34 and N-terminally positioned of the NGF antigen. In a further very preferred embodiment, said antigen consists of SEQ ID NO:30.

In a further embodiment, said antigen is feline NGF. In a further embodiment, said antigen comprises, or preferably consists of, SEQ ID NO:55 or an amino acid sequence having a sequence identity of at least 90% or at least 91%, preferably of at least 92%, at least 93% or at least 94%, further preferably of at least 95%, at least 96% or at least 97%, and further preferably of at least 98% or at least 99% amino acid sequence identity with SEQ ID NO: 55. In a further embodiment, said antigen comprises SEQ ID NO:55. In a further embodiment, said antigen consists of SEQ ID NO:55.

In a further embodiment, said antigen is equine NGF. In a further very preferred embodiment, said antigen comprises, or preferably consists of, SEQ ID NO:56 or an amino acid sequence having a sequence identity of at least 90% or at least 91%, preferably of at least 92%, at least 93% or at least 94%, further preferably of at least 95%, at least 96% or at least 97%, and further preferably of at least 98% or at least 99% amino acid sequence identity with SEQ ID NO:56. In a further embodiment, said antigen comprises SEQ ID NO:56. In a further preferred embodiment, said antigen consists of SEQ ID NO:56.

In a further embodiment, said antigen is bovine NGF. In a further embodiment, said antigen comprises, or preferably consists of, SEQ ID NO:57 or an amino acid sequence having a sequence identity of at least 90% or at least 91%, preferably of at least 92%, at least 93% or at least 94%, further preferably of at least 95%, at least 96% or at least 97%, and further preferably of at least 98% or at least 99% amino acid sequence identity with SEQ ID NO: 57. In a further embodiment, said antigen comprises SEQ ID NO:57. In a further preferred embodiment, said antigen consists of SEQ ID NO:57.

In a further embodiment, said antigen is porcine NGF. In a further embodiment, said antigen comprises, or preferably consists of, SEQ ID NO:58 or an amino acid sequence having a sequence identity of at least 90% or at least 91%, preferably of at least 92%, at least 93% or at least 94%, further preferably of at least 95%, at least 96% or at least 97%, and further preferably of at least 98% or at least 99% amino acid sequence identity with SEQ ID NO: 58. In a further embodiment, said antigen comprises SEQ ID NO:58. In a further preferred embodiment, said antigen consists of SEQ ID NO:58.

Without being bound, we believe that undesired aggregation and formation of aggregated conjugated CMV VLPs can in particular be reduced and avoided for antigens having a higher isoelectric point, and thus for antigens, which under the conditions used for conjugation would have an overall positive charge. Thus, in a preferred embodiment, said NGF antigen has an isoelectric point of above 6.5. In a preferred embodiment, said NGF antigen has an isoelectric point above 6.5 and below 13.0, preferably below 12.5, and further preferably below 12.0. In a preferred embodiment, said NGF antigen has an isoelectric point above 6.5, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said NGF antigen has an isoelectric point above 6.5 and below 13.0, preferably below 12.5, and further preferably below 12.0, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said NGF antigen has an isoelectric point of above 6.6, 6.7, 6.8 or 6.9. In a preferred embodiment, said NGF antigen has an isoelectric point above 6.6, 6.7, 6.8 or 6.9 and below 13.0, preferably below 12.5, and further preferably below 12.0. In a preferred embodiment, said NGF antigen has an isoelectric point of above 6.6, 6.7, 6.8 or 6.9, as determined by the ExPASy Compute pI/MW tool. In a preferred embodiment, said NGF antigen has an isoelectric point above 6.6, 6.7, 6.8 or 6.9 and below 13.0, preferably below 12.5, and further preferably below 12.0, as determined by the ExPASy Compute pI/MW tool. In a preferred embodiment, said NGF antigen has an isoelectric point of equal to or above 7.0. In a preferred embodiment, said NGF antigen has an isoelectric point equal or above 7.0 and below 13.0, preferably below 12.5, and further preferably below 12.0. In a preferred embodiment, said NGF antigen has an isoelectric point equal to or above 7.0, as determined by the ExPASy Compute pI/MW tool. In a preferred embodiment, said NGF antigen has an isoelectric point equal or above 7.0 and below 13.0, preferably below 12.5, and further preferably below 12.0, as determined by the ExPASy Compute pI/MW tool. In a preferred embodiment, said NGF antigen has an isoelectric point equal to or above 7.1, 7.2, 7.3 or 7.4. In a preferred embodiment, said NGF antigen has an isoelectric point equal to or above 7.1, 7.2, 7.3 or 7.4 and below 13.0, preferably below 12.5, and further preferably below 12.0. In a preferred embodiment, said NGF antigen has an isoelectric point equal to or above 7.1, 7.2, 7.3 or 7.4, as determined by the ExPASy Compute pI/MW tool. In a preferred embodiment, said NGF antigen has an isoelectric point equal to or above 7.1, 7.2, 7.3 or 7.4 and of below 13.0, preferably below 12.5, and further preferably below 12.0, as determined by the ExPASy Compute pI/MW tool. In a preferred embodiment, said NGF antigen has an isoelectric point equal to or above 7.5. In a preferred embodiment, said NGF antigen has an isoelectric point equal to or above 7.5 and below 13.0, preferably below 12.5, and further preferably below 12.0. In a preferred embodiment, said NGF antigen has an isoelectric point equal to or above 7.5, as determined by the ExPASy Compute pI/MW tool. In a preferred embodiment, said antigen has an isoelectric point equal to or above 7.5 and below 13.0, preferably below 12.5, and further preferably below 12.0, as determined by the ExPASy Compute pI/MW tool. In a preferred embodiment, said NGF antigen has an isoelectric point of equal or above 7.6, 7.7, 7.8 or 7.9. In a preferred embodiment, said NGF antigen has an isoelectric point equal or above 7.6, 7.7, 7.8 or 7.9 and below 13.0, preferably below 12.5, and further preferably below 12.0. In a preferred embodiment, said NGF antigen has an isoelectric point of equal or above 7.6, 7.7, 7.8 or 7.9, as determined by the ExPASy Compute pI/MW tool. In a preferred embodiment, said NGF antigen has an isoelectric point equal or above 7.6, 7.7, 7.8 or 7.9 and below 13.0, preferably below 12.5, and further preferably below 12.0, as determined by the ExPASy Compute pI/MW tool. In a preferred embodiment, said NGF antigen has an isoelectric point of equal or above 8.0. In a preferred embodiment, said NGF antigen has an isoelectric point equal or above 8.0, and below 13.0, preferably below 12.5, and further preferably below 12.0. In a preferred embodiment, said NGF antigen has an isoelectric point of equal or above 8.0, as determined by the ExPASy Compute pI/MW tool. In a preferred embodiment, said NGF antigen has an isoelectric point equal or above 8.0 and below 13.0, preferably below 12.5, and further preferably below 12.0, as determined by the ExPASy Compute pI/MW tool. In a preferred embodiment, said NGF antigen has an isoelectric point of equal or above 8.1, 8.2, 8.3 or 8.4. In a preferred embodiment, said NGF antigen has an isoelectric point equal or above 8.1, 8.2, 8.3 or 8.4, and below 13.0, preferably below 12.5, and further preferably below 12.0. In a preferred embodiment, said NGF antigen has an isoelectric point of equal or above 8.1, 8.2, 8.3 or 8.4, as determined by the ExPASy Compute pI/MW tool. In a preferred embodiment, said NGF antigen has an isoelectric point equal to or above 8.1, 8.2, 8.3 or 8.4 and below 13.0, preferably below 12.5, and further preferably below 12.0, as determined by the ExPASy Compute pI/MW tool. In a preferred embodiment, said NGF antigen has an isoelectric point equal to or above 8.5. In a preferred embodiment, said NGF antigen has an isoelectric point equal to or above 8.5, and below 13.0, preferably below 12.5, and further preferably below 12.0. In a preferred embodiment, said NGF antigen has an isoelectric point of equal or above 8.5, as determined by the ExPASy Compute pI/MW tool. In a preferred embodiment, said NGF antigen has an isoelectric point equal to or above 8.5 and below 13.0, preferably below 12.5, and further preferably below 12.0, as determined by the ExPASy Compute pI/MW tool.

In a very preferred embodiment, said polypeptide comprising said stretch of consecutive negative amino acids comprises SEQ ID NO:49, SEQ ID NO:50 or SEQ ID NO: 51. In a further very preferred embodiment, said polypeptide consists of SEQ ID NO:49, SEQ ID NO:50 or SEQ ID NO:51. In a further very preferred embodiment, said polypeptide comprises SEQ ID NO:49. In a further very preferred embodiment, said polypeptide comprises SEQ ID NO:50. In a further very preferred embodiment, said polypeptide comprises SEQ ID NO:51. In a further very preferred embodiment, said polypeptide consists of SEQ ID NO:49. In a further very preferred embodiment, said polypeptide consists of SEQ ID NO: 50. In a further very preferred embodiment, said polypeptide consists of SEQ ID NO: 51.

In a very preferred embodiment, said CMV polypeptide comprises the amino acid sequence of SEQ ID NO:5, SEQ ID NO:39 or SEQ ID NO:48, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:39, or between amino acid residues of position 86 (Ser) and position 87 (Thr) of SEQ ID NO:48. In a very preferred embodiment, said CMV polypeptide consists of the amino acid sequence of SEQ ID NO:5, SEQ ID NO:39 or SEQ ID NO:48, wherein said polypeptide comprising said stretch of consecutive negative amino acids is inserted between amino acid residues of position 88 and position 89 of said SEQ ID NO:5, between amino acid residues of position 84 and position 85 of SEQ ID NO:39, or between amino acid residues of position 86 and position 87 of SEQ ID NO: 48. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:39, wherein said polypeptide is inserted between amino acid residues of position 84 and position 85 of SEQ ID NO: 39. In a very preferred embodiment, said CMV polypeptide comprises the amino acid sequence of SEQ ID NO:48, wherein said polypeptide is inserted between amino acid residues of position 86 and position 87 of SEQ ID NO:48. In a very preferred embodiment, said CMV polypeptide consists of the amino acid sequence of SEQ ID NO:5, wherein said polypeptide is inserted between amino acid residues of position 88 and position 89 of SEQ ID NO: 5. In a very preferred embodiment, said CMV polypeptide consists of the amino acid sequence of SEQ ID NO:39, wherein said polypeptide is inserted between amino acid residues of position 84 and position 85 of SEQ ID NO:39. In a very preferred embodiment, said CMV polypeptide consists of the amino acid sequence of SEQ ID NO:48, wherein said polypeptide is inserted between amino acid residues of position 86 and position 87 of SEQ ID NO: 48.

In a very preferred embodiment, said chimeric CMV polypeptide comprises the amino acid sequence of SEQ ID NO:5, SEQ ID NO:39 or SEQ ID NO:48, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:39, or between amino acid residues of position 86 (Ser) and position 87 (Thr) of SEQ ID NO:48. In a very preferred embodiment, said chimeric CMV polypeptide consists of the amino acid sequence of SEQ ID NO: 5, SEQ ID NO:39 or SEQ ID NO:48, wherein said polypeptide comprising said stretch of consecutive negative amino acids is inserted between amino acid residues of position 88 and position 89 of said SEQ ID NO:5, between amino acid residues of position 84 and position 85 of SEQ ID NO:39, or between amino acid residues of position 86 and position 87 of SEQ ID NO:48. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:39, wherein said polypeptide is inserted between amino acid residues of position 84 and position 85 of SEQ ID NO:39. In a very preferred embodiment, said chimeric CMV polypeptide comprises the amino acid sequence of SEQ ID NO:48, wherein said polypeptide is inserted between amino acid residues of position 86 and position 87 of SEQ ID NO:48. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:39, wherein said polypeptide is inserted between amino acid residues of position 84 and position 85 of SEQ ID NO:39. In a very preferred embodiment, said chimeric CMV polypeptide consists of the amino acid sequence of SEQ ID NO:48, wherein said polypeptide is inserted between amino acid residues of position 86 and position 87 of SEQ ID NO: 48.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine, or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO: 12. In a very preferred embodiment, said chimeric CMV polypeptide comprises the amino acid sequence of SEQ ID NO: 10. In a very preferred embodiment, said chimeric CMV polypeptide comprises the amino acid sequence of SEQ ID NO: 11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO: 10, SEQ ID NO:11 or SEQ ID NO:12. In a very preferred embodiment, said chimeric CMV polypeptide consists of the amino acid sequence of SEQ ID NO: 10. In a very preferred embodiment, said chimeric CMV polypeptide consists of the amino acid sequence of SEQ ID NO: 11. In a very preferred embodiment, said chimeric CMV polypeptide consists of the amino acid sequence of SEQ ID NO:12.

In a very preferred embodiment, said modified VLP of CMV comprises 180 copies of said chimeric CMV polypeptide comprising the amino acid sequence of SEQ ID NO:10, SEQ ID NO:11 or SEQ ID NO:12. In a very preferred embodiment, said modified VLP of CMV comprises 180 copies of said chimeric CMV polypeptide comprising the amino acid sequence of SEQ ID NO:10. In a very preferred embodiment, said modified VLP of CMV comprises 180 copies of said chimeric CMV polypeptide comprising the amino acid sequence of SEQ ID NO:11. In a very preferred embodiment, said modified VLP of CMV comprises 180 copies of said chimeric CMV polypeptide comprising the amino acid sequence of SEQ ID NO:12. In a very preferred embodiment, said modified VLP of CMV comprises 180 copies of said chimeric CMV polypeptide consisting of the amino acid sequence of SEQ ID NO:10, SEQ ID NO:11 or SEQ ID NO:12. In a very preferred embodiment, said modified VLP of CMV comprises 180 copies of said chimeric CMV polypeptide consisting of the amino acid sequence of SEQ ID NO:10. In a very preferred embodiment, said modified VLP of CMV comprises 180 copies of said chimeric CMV polypeptide consisting of the amino acid sequence of SEQ ID NO:11. In a very preferred embodiment, said modified VLP of CMV comprises 180 copies of said chimeric CMV polypeptide consisting of the amino acid sequence of SEQ ID NO:12.

In a further very preferred embodiment, said antigen is canine NGF. In a very preferred embodiment, said antigen comprises, or preferably consists of, SEQ ID NO:30, SEQ ID NO: 31, SEQ ID NO:33 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30 or SEQ ID NO:31 or SEQ ID NO:33.

In further aspect, the present invention provides for a composition for use in a method of treating a NGF-related disorder in canine comprising the administration of said composition to said canine, wherein the composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO: 10;
(b) at least one antigen, wherein said antigen comprises at least one second attachment site, and wherein said antigen is a nerve growth factor (NGF) antigen; and wherein said antigen comprises, or preferably consists of, SEQ ID NO:30, SEQ ID NO:31, SEQ ID NO:33 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30 or SEQ ID NO:31 or SEQ ID NO:33; and
wherein (a) and (b) are linked, preferably by way of a heterobifunctional cross-linker through said at least one first and said at least one second attachment site via at least one covalent non-peptide bond, wherein said preferred hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group that has been chemically attached to the NGF antigen.

In a very preferred embodiment, said chimeric CMV polypeptide consists of the amino acid sequence of SEQ ID NO:10. In a very preferred embodiment, said modified VLP of CMV comprises 180 identical chimeric CMV polypeptides, wherein said chimeric CMV polypeptide comprises, preferably consists of, the amino acid sequence of SEQ ID NO:10. In a very preferred embodiment, said modified VLP of CMV comprises 180 identical chimeric CMV polypeptides, wherein said chimeric CMV polypeptide consists of the amino acid sequence of SEQ ID NO:10.

In a further preferred embodiment, said antigen comprises SEQ ID NO:30 or SEQ ID NO: 31 or SEQ ID NO:33. In a further preferred embodiment, said antigen consists of SEQ ID NO: 30 or SEQ ID NO:31 or SEQ ID NO:33. In a further very preferred embodiment, said antigen comprises, or preferably consists of, SEQ ID NO:30 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30. In a further very preferred embodiment, said antigen comprises SEQ ID NO:30. In a further very preferred embodiment, said antigen consists of SEQ ID NO:30. In a further preferred embodiment, said antigen comprises, or preferably consists of, SEQ ID NO:31 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:31. In a further very preferred embodiment, said antigen comprises SEQ ID NO:31. In a further very preferred embodiment, said antigen consists of SEQ ID NO:31. In a further very preferred embodiment, said antigen consists of SEQ ID NO:33. In a further preferred embodiment, said antigen comprises, or preferably consists of, SEQ ID NO:33 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:33. In a further very preferred embodiment, said antigen comprises SEQ ID NO:33. In a further very preferred embodiment, said antigen consists of SEQ ID NO:33. In a very preferred embodiment, said modified VLP of CMV comprises at least one, preferably 180 copies of said chimeric CMV polypeptide comprising the amino acid sequence of SEQ ID NO:10, SEQ ID NO:11 or SEQ ID NO:12, and said antigen comprises, or preferably consists of, SEQ ID NO:30 or SEQ ID NO:31 or SEQ ID NO:33 or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30 or SEQ ID NO:31 or SEQ ID NO: 33, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO: 10, said antigen comprises, or preferably consists of, SEQ ID NO:30, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:30, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:10, said antigen comprises, or preferably consists of, SEQ ID NO:31, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:31, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:31, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:31, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:30, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:30, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:31, or an amino acid sequence having a sequence identity of at least 90%, preferably of at least 91% or 92%, further preferably of at least 93%, 94% or at least 95%, and further preferably of at least 96%, 97% or at least 98% or at least 99% amino acid sequence identity with SEQ ID NO:31, and preferably all of said first attachments sites are not comprised or are not part of the polypeptide comprising said stretch of consecutive negative amino acids.

The modified CMV VLPs as described herein can be prepared in prokaryotic or eukaryotic expression systems. Preferred systems are E. coli, yeast, insect cells as well as mammalian cell lines. Very preferred said modified VLP of CMV or said VLP of CMV is obtained by expression of said chimeric CMV polypeptide in E. coli, and wherein preferably said expression is effected at temperatures of between 10° C. to 35° C.

Therefore, in another aspect, the present invention provides for a composition for use in a method of treating a NGF-related disorder in canine comprising the administration of said composition to said canine, wherein the composition comprises
(a) a modified virus-like particle (VLP) of cucumber mosaic virus (CMV) comprising at least one chimeric CMV polypeptide, wherein said at least one chimeric CMV polypeptide comprises, preferably consists of
   (i) a CMV polypeptide, wherein said CMV polypeptide comprises a coat protein of CMV or an amino acid sequence having a sequence identity of at least 75% with SEQ ID NO:39; and
   (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:39; and
(b) at least one antigen, wherein said antigen comprises at least one second attachment site, and wherein said antigen is a nerve growth factor (NGF) antigen; and
wherein (a) and (b) are linked through said at least one first and said at least one second attachment site, preferably via at least one covalent non-peptide bond, and wherein said modified VLP of CMV is obtained by expression of said chimeric CMV polypeptide in E. coli., and wherein preferably said expression is effected at temperatures of between 10° C. to 35° C.
said the modified virus-like particle (VLP) of cucumber mosaic virus (CMV) for use in a method of treating a NGF-related disorder in canine are purified from a recombinant bacterial host expressing said modified VLP of CMV, (a) lysing said bacterial host; (b) clarifying the lysate obtained by said lysis; (c) purifying said modified VLP of CMV from the clarified lysate by anion exchange chromatography (AEX); wherein said steps are performed in the given order.

In a further embodiment, said NGF containing CMV VLP composition comprises an adjuvant. Typical and preferred adjuvants are mineral salts (e.g. Aluminium Hydroxide, Aluminium Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). In a preferred embodiment, said composition comprises an adjuvant, wherein said adjuvant is aluminium hydroxide. In another preferred embodiment, said composition is devoid of an adjuvant.

Thus, the present invention provides for a composition for use in a method of treating a NGF-related disorder in canine comprising the administration of said composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen;
(c) optionally, or even preferably a T helper cell epitope; and
   wherein (a) and (b) are linked through said at least one first and said at least one second attachment site;
and wherein said composition further comprises an adjuvant. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. Preferably said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. Preferably, said (NGF-containing CMV VLP) composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

In a further aspect, the present invention provides vaccines for treating a NGF-related disorder in canine, preferably said vaccines are veterinary vaccines comprising, or alternatively consisting of, the NGF-containing CMV VLPs described herein. Encompassed are vaccines for use in a method of treating NGF-related disorder in canine, wherein said NGF-contain CMV VLP composition comprise any one of the technical features disclosed herein, either alone or in any possible combination. In a embodiment, the vaccine further comprises an adjuvant. In a embodiment, said vaccine comprises an adjuvant, wherein said adjuvant is aluminium hydroxide. Preferably, said NGF-related disorder is pain, more preferably pain selected from the group consisting of nociceptive pain, inflammatory pain, postsurgical pain, pain associated with musculoskeletal diseases, pain associated with degenerative joint disease and/or osteoarthritis (OA)-associated pain, more preferably acute pain associated with degenerative joint disease, chronic pain associated with degenerative joint disease, or even chronic refractory pain associated with degenerative joint disease, more preferably acute OA-associated pain, chronic OA-associated pain or even chronic refractory OA-associated pain. Preferably said (NGF-containing CMV VLP) composition is administered in an amount of 50 to 300 μg/dose, preferably in an amount of preferably in an amount of 50 to 275 μg/dose, preferably in an amount of 50 to 250 μg/dose, preferably in an amount of 50 to 200 μg/dose, preferably in an amount of 50 to 175 μg/dose, preferably in an amount of 50 to 150 μg/dose, preferably in an amount of 62.5 to 300 μg/dose, preferably in an amount of preferably in an amount of 62.5 to 275 μg/dose, preferably in an amount of 62.5 to 250 μg/dose, preferably in an amount of 62.5 to 200 μg/dose, preferably in an amount of 62.5 to 175 μg/dose, preferably in an amount of 62.5 to 150 μg/dose, preferably in an amount of 75 to 300 μg/dose, preferably in an amount of preferably in an amount of 75 to 275 μg/dose, preferably in an amount of 75 to 250 μg/dose, preferably in an amount of 75 to 200 μg/dose, preferably in an amount of 75 to 175 μg/dose, preferably in an amount of 75 to 150 μg/dose, preferably in an amount of 100 to 300 μg/dose, preferably in an amount of preferably in an amount of 100 to 275 μg/dose, preferably in an amount of 100 to 250 μg/dose, preferably in an amount of 100 to 200 μg/dose, preferably in an amount of 100 to 175 μg/dose, preferably in an amount of 100 to 150 μg/dose. Preferably, said (NGF-containing CMV VLP) composition is systematically administered, preferably subcutaneously administered to canine in at least two doses, preferably with a time interval between the first and the second dose of 7 to 21 days, preferably of 14 to 21 days, more preferably with a time interval between the first and the second dose of 7, 8, 9, 10, 11, 12, 13, 14, 15, 16, 17, 18, 19, 20 or 21 days, more preferably of 14, 15, 16, 17, 18, 19, 20 or 21 days. In case of any further administration of said composition, such third, optional fourth and any further optional administration comprises or consists of a systemically administration, preferably a subcutaneously administration of said third, optional fourth and optional further dose, preferably in a time interval of two to six months to the corresponding previous administration, preferably in a time interval of four to six months to the corresponding previous administration.

### EXAMPLES

### Example 1

### Construction and Production of Surface Charge Modified CMV VLPs

Different chimeric CMV polypeptides in accordance with the present invention were prepared, and subsequently expressed leading to the inventive modified CMV VLPs.

Towards this end, chimeric CMV polypeptides comprising, in particular, different polypeptides of contiguous negative amino acids, namely polypeptides consisting of either 4, 8, or 12 glutamic acid residues (“E4”—SEQ ID NO:1; “E8”—SEQ ID NO:2; “E12”—SEQ ID NO:3) were prepared such that said glutamic acid residues were inserted between amino acid residues Ser (88) and Tyr (89) of the modified CMV polypeptide CMV-Ntt830 (SEQ ID NO:5). Said modified CMV polypeptide CMV-Ntt830 comprises the T helper cell epitope derived from tetanus toxoid TT830 (SEQ ID NO:6). The corresponding nucleic acid sequence (SEQ ID NO:7) coding for said modified CMV polypeptide CMV-Ntt830 was prepared as described in Example 3 of WO2016/062720A1.

The prepared chimeric CMV polypeptides further comprise linkers flanking the introduced E4, E8 and E12 polypeptides at both termini. In detail, said prepared chimeric CMV polypeptides either comprise a GGS-linker or a GGGS-linker (SEQ ID NO:8) directly at the N-terminus of the introduced E4, E8, and E12 polypeptides, and either a GGGSGS-linker (SEQ ID NO:9) or a CGGGSGS-linker (SEQ ID NO:4) directly at the C-terminus of the introduced E4, E8, and E12 polypeptides.

The resulting amino acid sequences of said prepared chimeric CMV polypeptides are named “CMV-Ntt830-E4”, “CMV-Ntt830-E8”, “CMV-Ntt830-E8*” and “CMV-Ntt830-E12” and have the amino acid sequences as follows:

“CMV-Ntt830-E4”:;
SEQ ID NO: 10
“CMV-Ntt830-E8”:;
SEQ ID NO: 11
“CMV-Ntt830-E8*”:;
SEQ ID NO: 12
“CMV-Ntt830-E12”:.
SEQ ID NO: 13

The corresponding nucleotide sequences of said preferred chimeric CMV polypeptides are as follows:

“CMV-Ntt830-E4”:;
SEQ ID NO: 14
“CMV-Ntt830-E8”:;
SEQ ID NO: 15
“CMV-Ntt830-E8*”:;
SEQ ID NO: 16
“CMV-Ntt830-E12”:.
SEQ ID NO: 17

First, the chimeric CMV polypeptide CMV-Ntt830-E8* was prepared. Hereby and in a first step the incorporation of the coding sequence for E8 including the flanking linkers into the modified CMV using PCR mutagenesis was effected. The PCR fragment coding for the E8 sequence including the flanking linkers as well as the 3′ end fragment of the modified CMV was amplified in two step PCR using the following oligonucleotides:
Forward: E8*-1F (SEQ ID NO:18)
Forward: E8*-2F (SEQ ID NO:19)
Reverse: CMcpR (SEQ ID NO:20).

Thus, a PCR reaction was carried out using E8*-1F/CMcpR oligonucleotides and pET-CMV-Ntt830 plasmid as template. The template pET-CMV-Ntt830 was prepared as described in Example 3 of WO2016/062720A1. The target PCR product was obtained after a second PCR using oligonucleotides E8*-2F/CMcpR and the PCR product from the first PCR. The resulting PCR product was cloned into helper vector pTZ57 (InsTAclone PCR Cloning Kit, Fermentas #K1214). PCR product-containing plasmid was amplified in E. coli XL1-Blue cells, and plasmid DNA was purified and sequenced using BigDye cycle-sequencing kit and an ABI Prism 3100 Genetic Analyzer (Applied Biosystems). As a result, the helper plasmid pTZ-CMV-E8*, without PCR errors, was obtained.

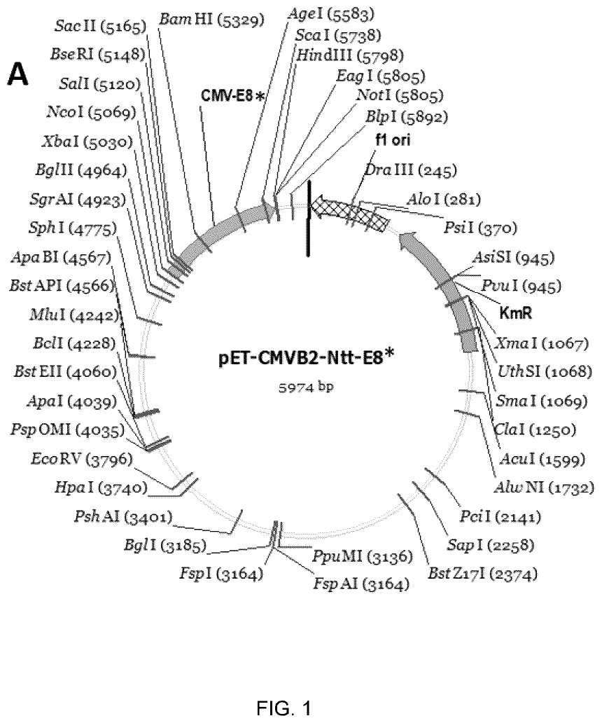

As a next step, the BamHI/HindIII fragment of pTZ-CMV-E8* was cloned back into the pET-CMV-Ntt830B helper vector using the same restriction sites, resulting in the expression vector pET-CMVB2-Ntt-E8C (FIG. 1).

The helper vector pET-CMV-Ntt830B was used for introduction of polypeptides comprising a stretch of consecutive negative amino acids coding DNA sequences in the corresponding CMV DNA sequence of CMV-Ntt830, BamHI site-containing sequence was introduced at the corresponding position for subsequent cloning. The CMV-Ntt830 coding nucleic acid sequence was prepared as described in Example 3 of WO2016/062720A1 and corresponds to SEQ ID NO:14 of WO2016/062720A1.

The BamHI site was introduced by two-step PCR mutagenesis using below listed oligonucleotides and previously constructed pET-CMV-Ntt830 as a template. As indicated, the template pET-CMV-Ntt830 was prepared as described in Example 3 of WO2016/062720A1.

1^{st} PCR: Forward-
(SEQ ID NO: 21
pET-90 primer (anneals pET28a+)
Reverse-
(SEQ ID NO: 22)
RGSYrev
2^{nd} PCR Forward-
(SEQ ID NO: 23)
RGSYdir
Reverse-
(SEQ ID NO: 24)
CMV-AgeR

After purification of both PCR products, the next PCR was carried out to join the PCR fragments (5 cycles without primers then 25 cycles using primers pET-90 and CMV-AgeR).

After amplification of the gene, the obtained PCR product was directly cloned into the pTZ57R/T vector (InsTAclone PCR Cloning Kit, Fermentas #K1214). E. coli XL1-Blue cells were used as a host for cloning and plasmid amplification.

To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone without sequence errors containing CMV-Ntt830B gene with introduced BamHI site was cut with NcoI and AgeI enzymes. Then the fragment was subcloned into the NcoI/AgeI sites of the pET-CMV-Ntt830, resulting in the helper vector pET-CMV-Ntt830B.

CMV-Ntt830-E8* VLPs were produced in E. coli C2566 cells (New England Biolabs, USA). The VLPs were produced using, E. coli cell cultivation, biomass treatment and purification methods as follows:
1) suspend 3 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);
2) Centrifuge the lysate at 11000 rpm for 20 min, at +4° C.;
3) Prepare sucrose gradient (20-60%) in 35 ml tubes, in buffer containing 50 mM Na citrate, 5 mM Na borate, 2 mM EDTA, 0.5% TX-100;
4) Overlay 5 ml of the VLP sample over the sucrose gradient;
5) Centrifuge 6 h using SW32 rotor, Beckman (25000 rpm, at +18° C.).
6) Divide the content of each gradient tube in 6 ml fractions. Pool corresponding fractions;
7) Analyse gradient fractions on SDS.

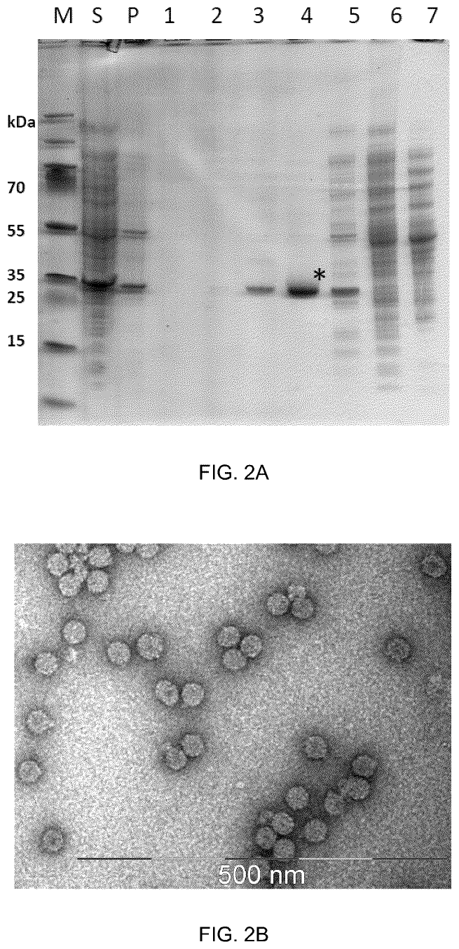

SDS-PAGE analysis of the VLPs after sucrose gradient purification demonstrates homogeneous CMV-Ntt830-E8* coat protein monomer (FIG. 2A) and electron microscopy shows intact VLPs (FIG. 2B).

The chimeric CMV polypeptides CMV-Ntt830-E4, CMV-Ntt830-E8 and CMV-Ntt830-E12 were prepared accordingly and as follows. The first step was the incorporation of the poly-glutamate coding sequences including the flanking linkers into the modified CMV using PCR mutagenesis. The PCR fragments coding for poly-glutamate sequences including the flanking linkers as well as the 3′ end fragment of the modified CMV were amplified by PCR using the following pairs of oligonucleotides and plasmid pET-CMVB2-Ntt-E8* as a template:

1) Forward
(SEQ ID NO: 25)
E4-F
Reverse:
(SEQ ID NO: 20)
CMcpR;
2) Forward:
(SEQ ID NO: 26)
E8-F
Reverse:
(SEQ ID NO: 20)
CMcpR;
3) Forward:
(SEQ ID NO: 27)
E12-F
Reverse:
(SEQ ID NO: 20)
CMcpR.

The resulting PCR products were cloned into helper vector pTZ57 (InsTAclone PCR Cloning Kit, Fermentas #K1214). PCR product containing plasmids were amplified in E. coli XL1-Blue cells, and plasmid DNAs purified and sequenced using BigDye cycle-sequencing kit and an ABI Prism 3100 Genetic Analyzer (Applied Biosystems). Thus the helper plasmids pTZ-CMV-E4, pTZ-CMV-E8 and pTZ-CMV-E12 without PCR errors were obtained.

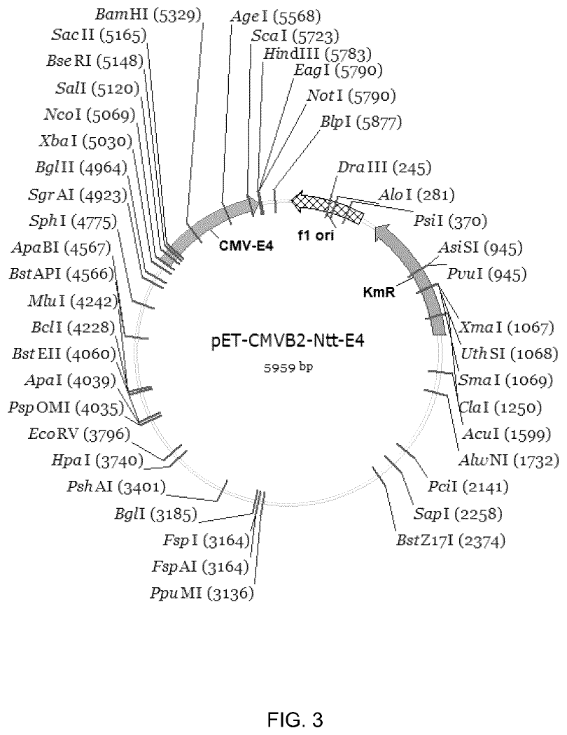

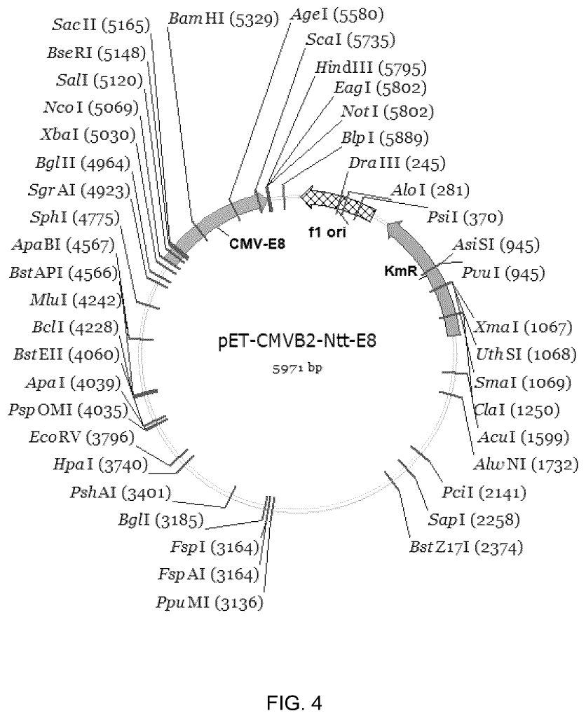

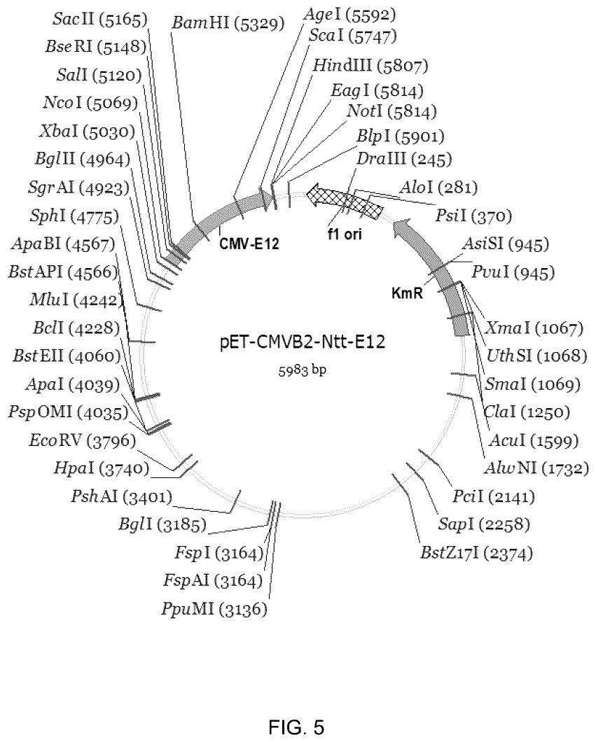

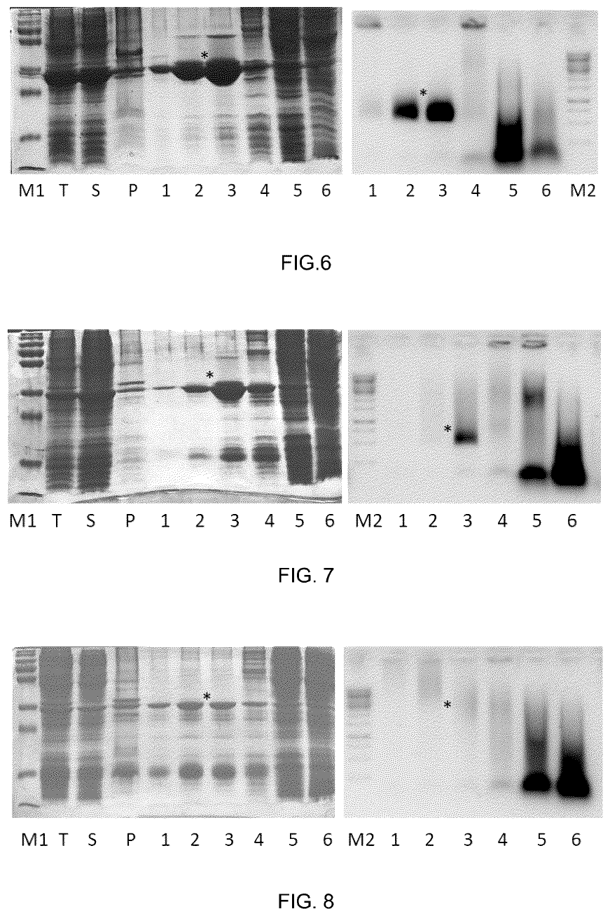

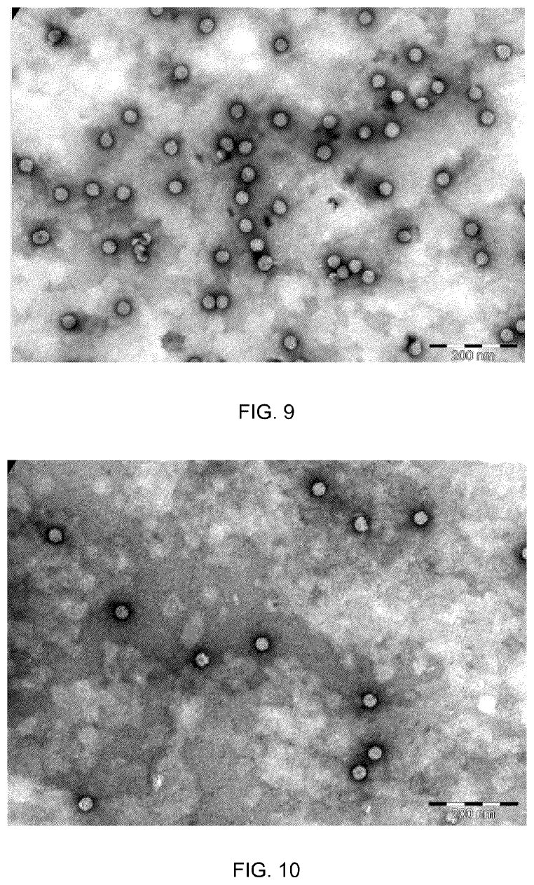

Next, the BamHI/HindIII digested fragments of pTZ-CMV-E4, pTZ-CMV-E8 and pTZ-CMV-E12 were cloned back into the pET-CMV-Ntt830B (see above) using the same restriction sites. The expression vectors pET-CMVB2-Ntt-E4 (FIG. 3) pET-CMVB2-Ntt-E8 (FIG. 4), and pET-CMVB2-Ntt-E12 (FIG. 5) were thus obtained. The expression vectors were transformed into E. coli C2566 cells (New England Biolabs, USA). VLPs were produced using, E. coli cell-cultivation, biomass-treatment and purification methods as described above for CMV-Ntt830-E8* VLPs. SDS-PAGE analyses of the VLPs after sucrose gradient purification demonstrated near homogeneous CMV-coat protein monomer was obtained for all 3 poly-glutamate constructs (FIG. 6, FIG. 7, FIG. 8). However, agarose gel analysis showed integral particles were only formed with CMV-Ntt830-E4 and CMV-Ntt830-E8 but not with the CMV-E12 (FIG. 6, FIG. 7, FIG. 8). Electron microscopy showed that CMV-Ntt830-E4 and CMV-Ntt830-E8 formed intact VLPs (FIG. 9, FIG. 10).

### Example 2

### Stability of the Surface Charge Modified CMV VLPs as Compared to Non-Surface Charged CMV VLPs

### Thermal Stability

Increased thermal stability of the inventive surface charge modified CMV VLPs was demonstrated by measuring denaturation of the prior art CMV-Ntt830 VLPs, which were prepared as described in Example 3 and Example 4 of WO2016/062720A1, and of the inventive CMV-Ntt830-E4 VLPs as a function of increasing temperature and determining the respective melting points.

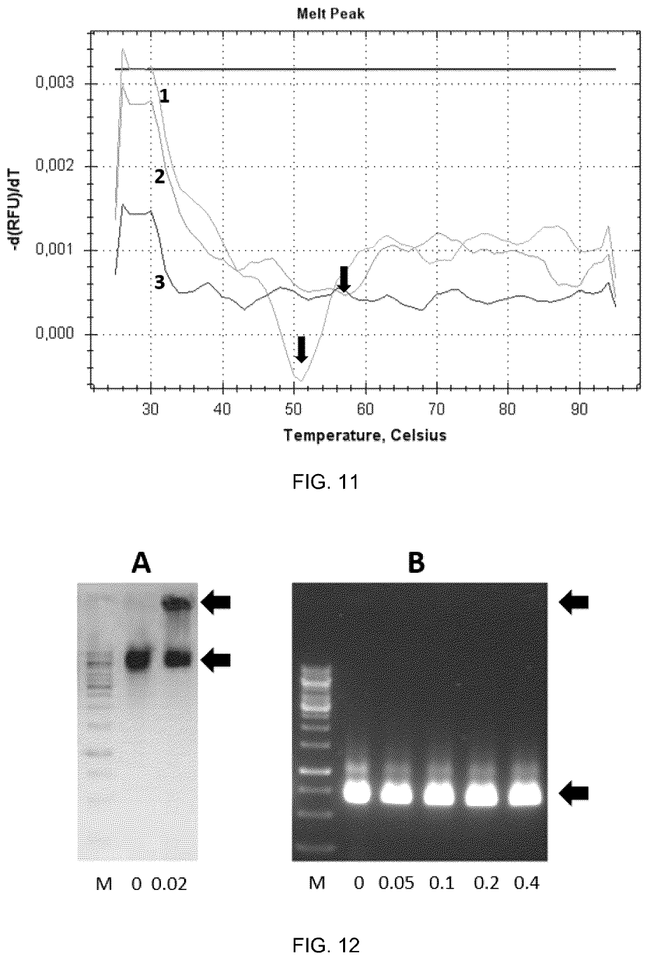

A thermal shift assay involving temperature-induced denaturation and the fluorescent dye SYPRO® Orange (Sigma, Saint Louis, USA) was used for this purpose. The dye is a naturally quenched in solution but as the VLPs denature with increasing temperatures, SYPRO® Orange interacts with exposed hydrophobic amino acids and cores and emits a fluorescent signal, which is measured by fluorometry. From the resultant melting curve (fluorescent signal vs temperature), the melt peak curves and melting temperature were determined. Solutions containing 0.5 mg/ml of sucrose density gradient purified (as described in Example 1 above) CMV-Ntt830 VLPs or CMV-Ntt830-E4 VLPs in 5 mM Na phosphate, 2 mM EDTA, pH 7.5 were assayed with a real-time PCR system MJ Mini (Bio-Rad, Hercules, USA) using a DNA melting point determination program. Data were analysed using Opticon Monitor Software and melting curves processed at a smooth setting of four. FIG. 11 shows the melt peak curves for purified CMV-Ntt830 VLPs and CMV-Ntt830-E4 VLPs.

The respective melting temperatures were estimated to be 51° C., and 57° C. evidencing an increased thermal stability of the surface charge modified CMV VLPs in accordance with the invention as compared to the prior art CMV-Ntt830 VLPs.

### Ionic Strength/Salt Stability

Ionic strength is important for capsid stability. Salts in solution interact with charged residues on the coat proteins and VLP surfaces, influence the water shell and disfavor hydrophobic exposure and thereby influence overall VLP stability.

The relative stabilities of CMV-Ntt830 VLPs and CMV-Ntt830-E4 VLPs to NaCl were tested by incubating purified VLPs (0.5 mg/ml in 5 mM Na phosphate, 2 mM EDTA, pH 7.5) at room temperature with various NaCl concentrations. After 2 hours in the presence of 20 mM NaCl, the CMV-Ntt830 VLPs were relatively unstable and formed aggregates in significant proportion that were both visible to the eye and demonstrable by native gel electrophoresis (FIG. 12). In contrast, there was no evidence of aggregate formation for CMV-Ntt830-E4 VLPs even with NaCl concentrations up to 0.4 M (FIG. 12).

The improved stability in higher salt solution arising from the surface charge modifications of the inventive modified CMV VLPs is important for its processability by ion-exchange chromatography as described in Example 3.

### Example 3

### Purification Potential of Surface Charge Modified CMV VLPs as Compared to Non-Surface-Charged CMV VLPs

The sucrose gradient/cushion ultra-centrifugation purification step, which was used in the laboratory-scale CMV VLP manufacture process as described in the prior art such as in Examples 2-4 of WO2016/062720A1 and for the preparation of the inventive modified CMV VLPs as described in Example 1 above, provides CMV VLPs of suitable yield and purity for subsequent conjugation, vaccine manufacture and preclinical evaluation. However, this method cannot be simply and cost effectively used to produce vaccine for commercial purposes.

Ion exchange chromatography (IEX) is typically readily scalable and used in downstream processes for the commercial production of biologics. It is based on reversible ionic interactions between charged molecules/macromolecules in solution and an immobilized oppositely charged chromatography resin. An example is anion-exchange chromatography (AEX) where the stationary phase (resin) is positively charged and negatively charged molecules such as proteins are bound. The interaction of the resin and sample can be disrupted by application of a counter ion such as Cl⁻. IEX is commonly used in bind/elute mode to provide rapid capture, high-resolution purification and concentration of the desired sample. It can be employed in the initial (e.g. after lysate clarification), intermediate or penultimate stages of a downstream process.

For CMV VLPs to be effectively bound and eluted by IEX, it is necessary that the CMV VLP is stable to the ionic environment encountered during the binding and elution phases. Both the charge on the ion-exchange resin and elution salt contribute to the ionic environment.

The prior art CMV-Ntt830 VLPs as well as the inventive modified CMV VLPs such as CMV-Ntt830-E4, CMV-Ntt830-E8 and CMV-Ntt830-E8* have a net negative charge at about pH's of 9 and below, as demonstrated by their migration towards the positively charged electrode in NAGE. Thus, anion-exchange chromatography (AIX) is a technique that would have been expected to work for both CMV VLP particles.

However, this is not the case because the CMV-Ntt830 VLPs, as described above in Example 2, are relatively unstable in solution in the presence of already 20 mM NaCl and form aggregates, which precipitate. In contrast, the inventive modified CMV VLPs such as the CMV-Ntt830-E4 VLP do not form aggregates at NaCl concentrations up to 0.4 M (FIG. 12, Panel B). The improved stability in higher salt solution arising from the surface charge modifications to the VLP is essential for its processability by ion-exchange chromatography.

### Purification by Anion Exchange Chromatography (AEX).

To test the processability of prior art CMV-Ntt830 VLPs with anion exchange chromatography (AEX), sucrose gradient purified VLPs were prepared as described in Examples 2-4 of WO2016/062720A1. Five mls of CMV-Ntt830 VLPs (1 mg/ml) were buffer exchanged into 5 mM sodium borate pH 9 and loaded onto a 1.0 ml Macro-Prep DEAE Bio-Rad anion exchange cartridge equilibrated with the same buffer. After the loading step, the concentration of NaCl in the elution buffer was increased in step-wise manner (0.1, 0.2, 0.3, 0.4, 0.5, 0.8., 1.0 and 2.0 M). Fractions were collected and measured at 260 nm using Nanodrop spectrophotometer to measure protein and subjected to native agarose gel electrophoresis (NAGE).

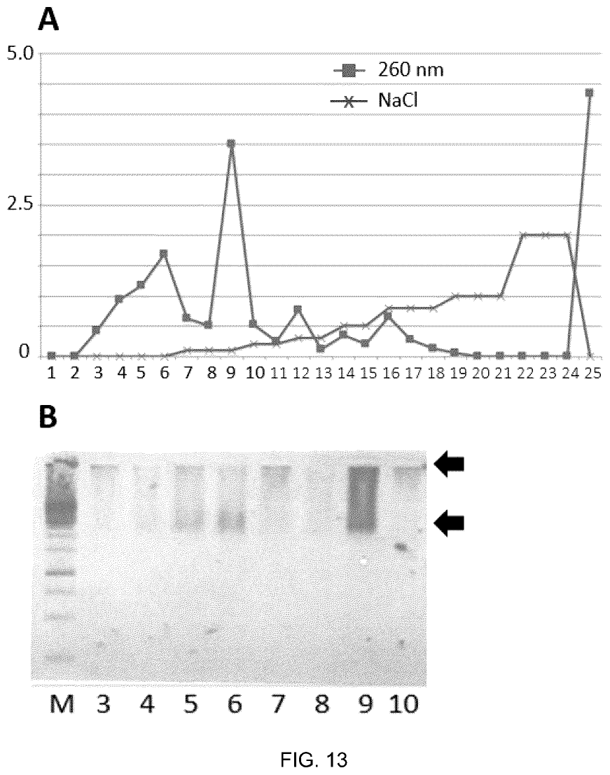

The resultant chromatogram of protein elution and NaCl concentrations plotted against the corresponding fraction (FIG. 13, panel A) shows the CMV-Ntt830 VLPs did not elute as a single peak as is typical for AIX. Instead, CMV-Ntt830 VLPs eluted in a broad non-specific manner during the loading (at 0 M NaCl) and subsequent elution steps over a range of NaCl concentrations, principally 0.2 to 0.8 M. Critically, the VLP-containing fractions after elution from the column were turbid and contained a significant proportion of aggregated VLPs, as demonstrated by the presence of ethidium bromide stained VLPs in the loading wells following NAGE (FIG. 13, panel B). The propensity of the CMV-Ntt830 VLPs to aggregate and elute in a non-discrete manner precludes the ready use of this methodology for scale-up manufacture.

In contrast, non-aggregated CMV-Ntt830-E4 VLPs could be readily purified from a crude lysate using AEX. Clarified lysate prepared from E. coli expressing CMV-Ntt830-E4 VLPs (as described in Example 1) in 50 mM citrate, 5 mM Borate buffer pH 9.0 was loaded onto 60 ml of Fracto-DEAE (Merck) in an XK 26/20 column equilibrated with the same buffer and eluted by applying a continuous NaCl gradient from 0 to 1.0 M in the same buffer. The eluate was monitored at A260 nm to measure protein and conductivity measured to monitor salt concentration. The clarified lysate, flow-through and fractions were collected and subjected to NAGE and SDS-PAGE.

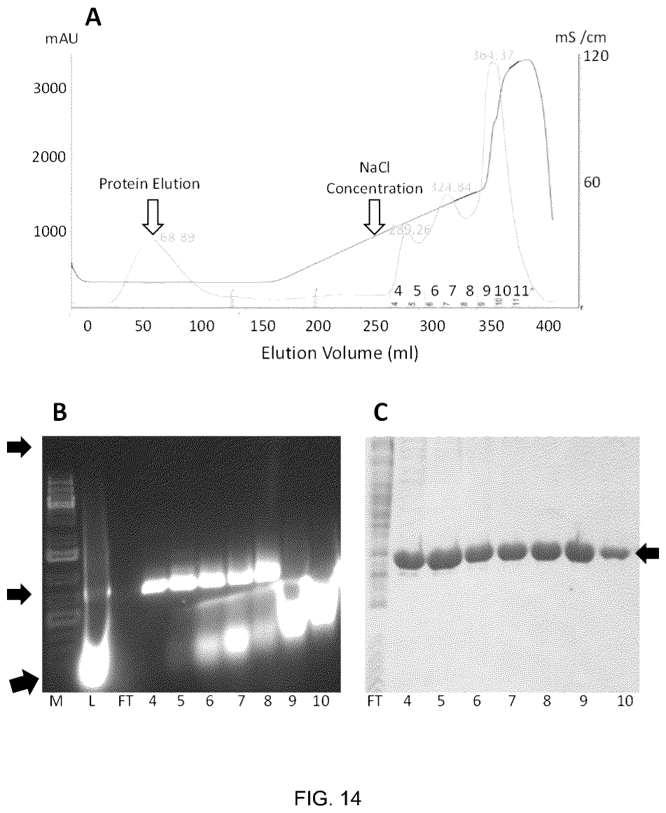

The resultant chromatogram, SDS-PAGE and NAGE analyses (FIG. 14) show that the CMV-Ntt830-E4 VLPs were not present in the flow-through and entirely bound to the Fracto-DEAE. The VLPs were subsequently eluted over a relatively narrow concentration range of 0.2-0.5M NaCl. Moreover, there was no evidence of aggregated VLPs in the loading wells of the native agarose gel. The Coomassie blue stained SDS-polyacrylamide gel showed highly pure VLP coat protein was obtained from the crude bacterial lysate.

### Example 4

### Cloning, Expression and Purification of Recombinant Mature NGF

### Cloning of Recombinant NGF

A cDNA construct (SEQ ID NO:28) consisting of full-length feline NGF pro-peptide sequence, canine mature NGF sequence and a C-terminal glycine-cysteine-glycine motif was synthesized de novo and cloned into pBHA vector (BIONEER Company). The canine NGF sequence was codon optimized. The resulting amino acid sequence of the full-length feline NGF pro-peptide is provided in SEQ ID NO:29 comprising the canine mature NGF sequence of SEQ ID NO:30. The amino acid sequence of canine mature NGF to which said C-terminal glycine-cysteine-glycine motif is attached is provided in SEQ ID NO:31.

Analogously, a cDNA construct (SEQ ID NO:32) consisting of full-length feline NGF pro-peptide sequence, canine mature NGF sequence, a C-terminal glycine-cysteine-glycine motif and a his-tag was synthesized de novo and cloned into pBHA vector (BIONEER Company). The included his-tag does not fulfil any roles for purification, but its presence increased refolding efficiency in downstream processes. The resulting amino acid sequence is provided in SEQ ID NO:33 comprising the canine mature NGF sequence of SEQ ID NO: 30 as well as the His6-tag (SEQ ID NO:34).

The constructs were sub-cloned into an expression vector by PCR. Briefly, the NGF-pBHA plasmid was used as a template with an NGF forward primer (SEQ ID NO:35), and an NGF reverse primer (SEQ ID NO:36), containing XbaI and HindIII sites respectively.

The NGF PCR product was subject to 1% agarose gel electrophoresis in TAE buffer and then NGF fragment extracted with GeneJet DNA elution kit (Thermo Fisher Scientific) according to the manufacturer's protocol. The NGF fragment was digested with FastDigest XbaI and HindIII (Thermo Fisher Scientific) restriction enzymes for 30 min in 1× FastDigest buffer at +37° C. according to the manufacturer's protocol. pET42a plasmid (Novagen) was digested in the same manner. The NGF and vector digested DNA fragments were analysed with agarose gel electrophoresis and extracted as above. The NGF fragment was ligated in the pET42a vector using T4 ligase overnight in room temperature according to manufacturer's protocol.

The NGF-pET42a construct was transformed in chemically competent E. coli DH5a cells by the heat shock method. The cells were suspended in 1 ml of LB medium and incubated at +37° C. with shaking for 1 hour and plated onto LB agar containing 60 μg/ml kanamycin and incubated overnight at 37° C. Individual colonies were seeded into LB medium, containing 30 μg/ml kanamycin and incubated overnight at +37° C. with shaking. DNA was extracted from individual clone cultures with GeneJet plasmid miniprep kit (Thermo Fisher Scientific) according to manufacturer protocol.

The correct sequence of the NGF constructs of SEQ ID NO:28 and SEQ ID NO:32 were confirmed by Sanger sequencing using a BigDye Terminator v3.1 Cycle Sequencing Kit (Thermo Fisher Scientific) according to manufacturer's protocol.

### Expression and Purification of Recombinant Canine Mature NGF

The NGF-pET42a plasmid was transformed into chemically competent E. coli BL21-DE3 (Sigma-Aldrich) cells. The cells were suspended in 1 ml of LB medium and incubated at +37° C. with shaking for 1 hour. The cells were plated onto LB agar containing 60 μg/ml kanamycin and incubated overnight at 37° C. Several colonies of NGF-pET42 transformed BL21-DE3 cells were seeded into LB medium containing 30 μg/ml kanamycin, and incubating overnight at 37° C., and then added to 2×TY medium containing 30 μg/ml kanamycin and grown at 37° C. with shaking until OD540 nm of 0.7 units was reached. Recombinant protein expression was induced by addition of IPTG to a final concentration of 1 mM and cells grown for an additional 4 hours at 37° C. with shaking. The biomass was collected by centrifugation at 5000 g for 15 minutes, frozen and stored at −70° C.

The biomass was suspended in lysis buffer (40 mM Tris-HCl (pH 8.0), 200 mM NaCl, 1 mM PMSF, 1 mM DTT and 1% Triton X-100) and cells lysed by sonication, using a UP200S (Hielscher) ultrasound device. The resultant sonicate was centrifuged for 40 min at 15 557 g. The supernatant was discarded and lysis buffer was added to the pellet which was Re-suspended by sonication. The suspension was centrifuged for 15 min at 15 557 g and the supernatant again discarded. This washing step was repeated three more times. The pellet was washed a final time with 50% lysis buffer and 3.5 M urea. After resuspension and centrifugation the pellet was solubilized with 8 M guanidine hydrochloride and 0.1 M dithiotreithol. The suspension was homogenized by sonication for 10 minutes then centrifuged for 25 min at 15 557 g. The supernatant (containing solubilized denatured NGF) was collected and filtered using a 45 μm filter then added dropwise into refolding buffer (0.75 M L-arginine, 0.1 M Tris, 1 mM EDTA, 5 mM reduced glutathione and 0.5 mM oxidized glutathione pH 9.5) at 7° C. with constant stirring to a final concentration of 5 ml of NGF solution per 100 ml of refolding buffer. After overnight incubation the refolding solution was centrifuged at 10 000 g for 10 minutes and the supernatant collected and incubated for one week at +7° C. The solution was diluted threefold with deionized water, warmed to room temperature and the pH adjusted to 6.8 with acetic acid. The solution was then centrifuged at 7 000 g for 10 minutes at room temperature to remove precipitates and loaded on a 5 ml Capto S cation exchange column, previously equilibrated with 50 mM sodium phosphate buffer (pH 6.5). The proteins were then eluted with a gradient of 0-1 M NaCl in 50 mM sodium phosphate buffer (pH 6.5). The eluted fractions were analyzed with SDS-PAGE and those containing proNGF were pooled and concentrated with ultrafiltration to 2-3 mg/ml. The renatured proNGF was digested with TrypZean (Sigma-Aldrich, cat no. T3449) trypsin solution for 4 hours at room with volume ratio of 30:1. The reaction was stopped by adding PMSF to final concentration of 1 mM, then loaded onto a Superdex 200 10/300 GL size exclusion column equilibrated with 0.5 M NaCl and 30 mM phosphate (pH 6.8).

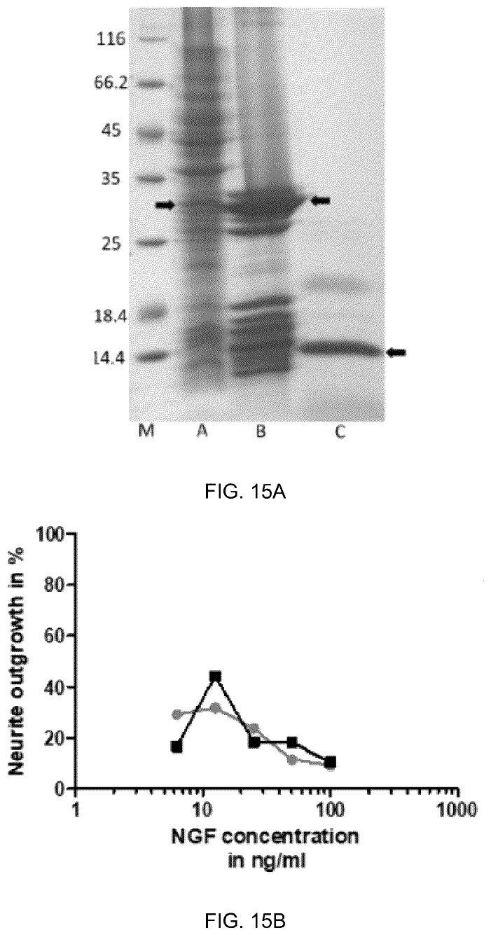

Fractions were collected and analysed with SDS-PAGE (FIG. 15A; shown for cDNA construct of SEQ ID NO:28 and resulting amino acid sequence of the full-length feline NGF pro-peptide of SEQ ID NO:29) and those containing the mature NGF were pooled and concentrated by ultrafiltration to a concentration of 2 mg/ml.

The authenticity of the recombinant canine mature NGF was confirmed using a bioassay which showed the canine mature NGF and mouse mature NGF (commercially produced by R&D systems) were similarly active at inducing neurite (FIG. 15B; shown for cDNA construct of SEQ ID NO:28 and resulting amino acid sequence of the full-length feline NGF pro-peptide of SEQ ID NO:29); a known function of properly folded and biologically active mature NGF.

### Example 5

### Coupling of Recombinant Canine Mature NGF to Modified CMV VLPs

Various NGF antigens comprising canine mature NGF (SEQ ID NO:30) were covalently linked to the various modified CMV VLPs prepared as described above. The linking was effected in accordance with the method described in Schmitz N, et al, J Exp Med (2009) 206:1941-1955).

Briefly, purified CMV-Ntt830, CMV-Ntt830-E4, CMV-Ntt830-E8 or CMV-Ntt830-E8* VLPs were diluted to 1.5 mg/ml and reacted with heterobifunctional chemical cross-linker succinimidyl-6-(b-maleimidopropionamide) hexanoate (SMPH) for 1 hour at room temperature (RT). SMPH contains a NHS ester which reacts with the lysine on the surface of the VLP. The amount of SMPH added was approximately 5× molar excess over one VLP coat protein monomer. Cross-linker which did not react with the VLP was removed by centrifugation using an Amicon-Ultra-0.5, 100K centrifugal filter (Merck-Millipore, #UFC910024). The SMPH-derivatized VLPs were then washed 3 times with 5 mM Na2HPO4, 2 mM EDTA (pH 7.5).

In detail, and described for the coupling of cNGF antigens having SEQ ID NO:33 to CMV-Ntt830-E4 VLPs: A solution of CMV-Ntt830-E4 VLPs in 5 mM NaHPO4 pH 7.5, 2 mM EDTA, —with a protein concentration of 7.43 mg/ml BCA Protein Assay Kit (TFS, Cat. No. 23225) was diluted to a working concentration of 1.5 mg/ml with 5 mM NaHPO4 pH 7.5, 2 mM EDTA pH 8.0 in 3×44 ml sample volume in 50 ml tubes (Sarstedt, sterile, Cat. No. 62.559.001), thus the total volume for derivatization was 132 ml. 50 mM (19 mg/ml) SMPH solution in DMSO was prepared directly before use.

For derivatization of CMV-Ntt830-E4 VLPs with SMPH, 264 μl of 50 mM SMPH solution in DMSO was added to each of previously prepared three tubes containing 44 ml of CMV-Ntt830-E4 VLPs. Mixture was vortexed for 5 seconds and incubated at RT for 1 h. To remove excess SMPH mixture was further centrifugated on Amicon-Ultra-15 100K units (Merck-Millipore, Cat. No. UFC910024) for 7 min at 3214 g in Eppendorf 5810R centrifuge. The buffer was exchanged to 5 mM NaHPO4 pH 7.5, 2 mM EDTA by 3 more centrifuge runs with same parameters. After the last centrifugation run the total volume was adjusted to 132 ml (same as before derivatization). UV absorption at 260 nm was measured and concentration of derivatized CMV-Ntt830-E4 VLPs was estimated at 1.5 mg/ml.

Next, and briefly, cNGF antigens were added to the VLPs in an about 0.5:1 to 1:1 molar ratio, with respect to the respective chimeric CMV polypeptide monomer, to the previously SMPH derivatized surface charge modified CMV VLPs for typically 3 hours at RT while shaking. The engineered free cysteine of the cNGF antigen reacted with the maleimide of the cross-linker SMPH bound to the VLPs to form a stable covalent linkage.

In detail, and described for the coupling of cNGF antigens having SEQ ID NO:33 to CMV-Ntt830-E4 VLPs: The coupling reaction were performed in six 50 ml tubes (Sarstedt, sterile, Cat. No. 62.559.001). In each tube 22 ml of derivatized CMV-Ntt830-E4 VLPs (1.5 mg/ml, 60 μM in respect to CMV monomers) was mixed with 3.82 ml of buffer-exchanged cNGF of SEQ ID NO:33 (2.33 mg/ml, 172.6 μM). This yielded to a molar ratio of CMV monomers: NGF monomers=1:0.5. The reaction mix was incubated at RT by end-over-end rotation with DSG Titertek (Flow Laboratories). Uncoupled cNGF was removed by gel-filtration on Superdex 200 column (run buffer 20 mM NaHPO4 pH 7.5, 2 mM EDTA). 10 ml of the solution comprising cNGF-CMV-Ntt830-E4 VLPs was loaded on a HiLoad 26/600 Superdex 200 prep grade column equilibrated in 20 mM NaHPO4 pH 7.5, 2 mM EDTA. The fractions containing cNGF-CMV-Ntt830-E4 VLPs were pooled and filtrated with filtered through a 0.2 μm filter (Sarstedt, Cat. No. 83.1826.001). Collected sample volume after gel-filtration was 280 ml. The sample was further concentrated by Amicon-Ultra-15, 100K (Merck-Millipore, Cat. No. UFC910024) to 230 ml and filtered through a 0.2 μm filter (Sarstedt, Cat. No. 83.1826.001). The concentration was measured by Qubit and the final concentration was adjusted to 0.7 mg/ml with sterile 20 mM NaHPO4 pH 7.5, 2 mM EDTA buffer. UV absorbance at 260 nm was measured (A230=6.628 and A260-3.722).

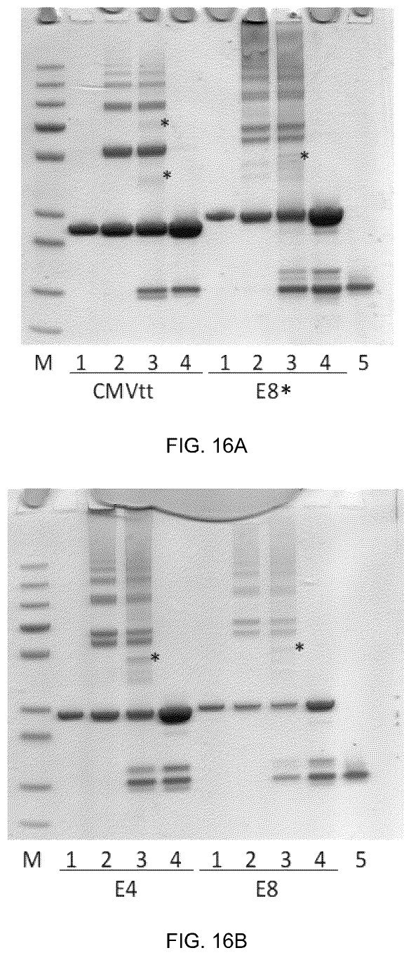

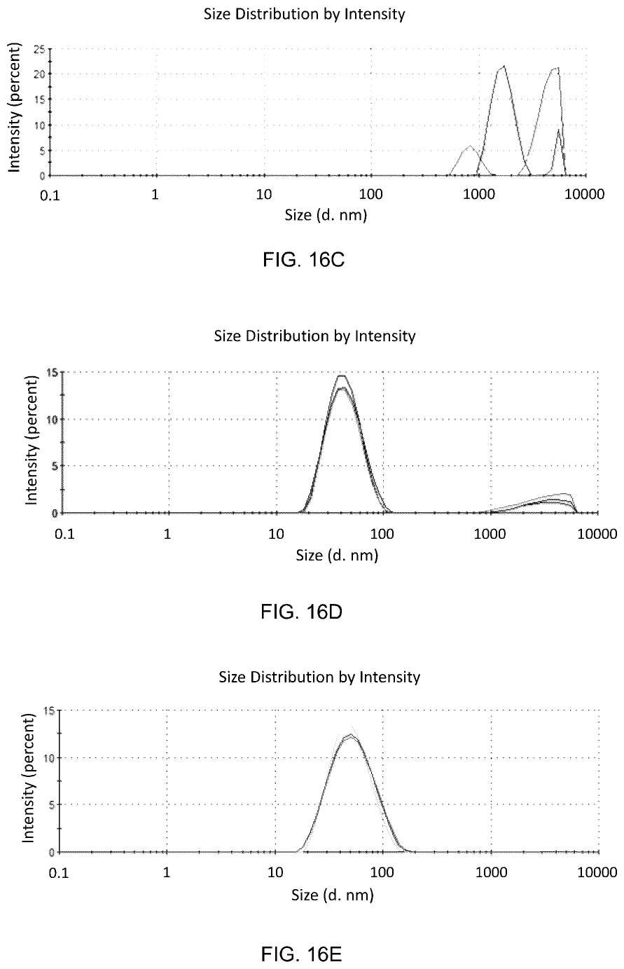

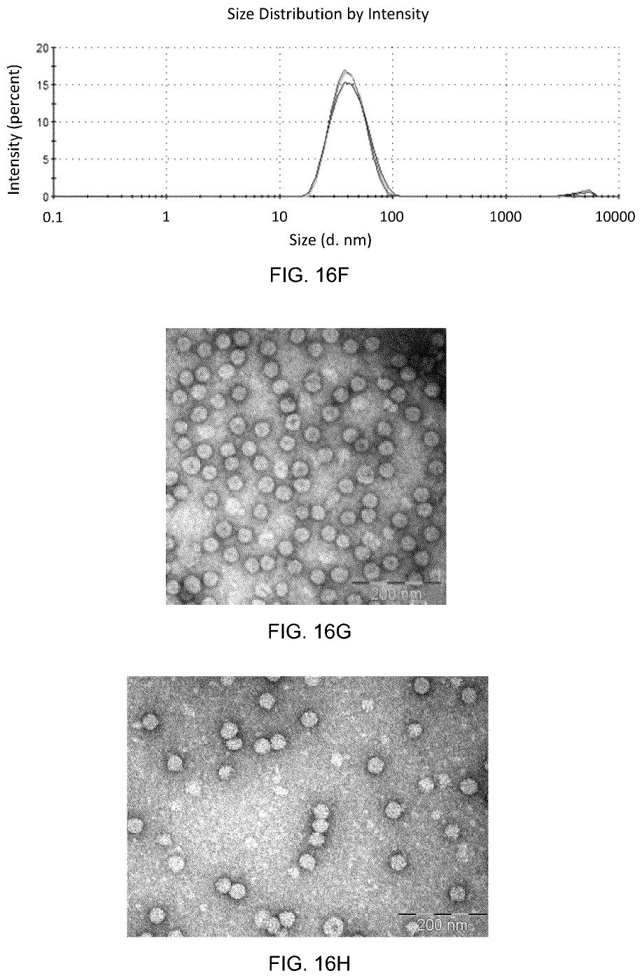

To demonstrate covalent conjugation of cNGF antigens to VLPs, coupling reactions were analyzed by SDS-PAGE. Prominent conjugation bands were observed following chemical coupling of cNGF with CMV-Ntt830, CMV-Ntt830-E4, CMV-Ntt830-E8 and CMV-Ntt830-E8* VLPs (FIG. 16A and FIG. 16B). However, cNGF-CMV-Ntt VLPs formed large aggregates (1400-1700 nm) (FIG. 16C) and rapidly and completely precipitated from solution.

In contrast, after the covalent conjugation of cNGF to the CMV-Ntt830-E4, CMV-Ntt830-E8 and CMV-Ntt830-E8* VLPs, the resultant modified VLP conjugates remained soluble and did not precipitate from solution. Analysis by dynamic light scattering (DLS) (FIG. 16D, FIG. 16E and FIG. 16F) and electron microscopy (FIG. 16G and FIG. 16H) showed the modified VLP conjugates were not aggregated and were stable in solution.

### Example 6

Induction of Neutralizing Antibodies by Immunizing with Various Inventive Conjugates of Canine Mature NGF Coupled to Modified CMV VLPs

### Immunization of Dogs

Six male Beagles aged 22-26 months at the time of first dosing (obtained from Marshall US) were assigned across 2 groups (n=3 per group) by randomization. The first group was immunized three times with 1.0 ml of cNGF-CMV-Ntt830-E8* VLP formulated to a concentration of 250 μg/ml in Na phosphate buffer, pH 7.5. The second group was similarly treated with cNGF-CMV-Ntt830-E8* VLP formulated to a concentration of 250 μg/ml in Na phosphate buffer, pH 7.5 and 100 μg/ml Quil-AR adjuvant (In vivoGen vac-quil). Blood specimens were drawn from the jugular vein with single use needles and syringes of each animal 24 hours before the first (day 0), second (Day 21) and third (Day 42) immunization. Blood was also drawn on days 63, 84 and 105. Six ml samples of blood were collected in inert tubes and left at ambient temperature. After clot formation, the tubes were centrifuged and serum collected into inert tubes and stored at ca. −20° C. until IgG purification and/or assayed.

In a further study, 10 adult Beagle dogs over the age of 9 months at inclusion were allocated into 2 groups. For immunization, cNGF-CMV-Ntt830-E4 VLPs comprising cNGF antigens of SEQ ID NO:33 were used. Thus, the first group of 5 dogs were treated with 250 μg cNGF-CMV-Ntt830-E4 VLPs/dose formulated with 1.7 mg aluminium hydroxide, while the second group of 5 dogs were treated with 250 μg cNGF-CMV-Ntt830-E4 VLPs/dose without aluminium hydroxide. Dogs were administered subcutaneously on two occasions on study days 0 and 21. Serum samples were also collected throughout the study on days 42, 71 and 91.

### Measurement of NGF and CMV-VLP Specific IgG Antibodies

For dogs immunized with cNGF-CMV-Ntt830-E8* VLP, anti-NGF- and CMV-Ntt830-E8*-VLP specific IgG antibodies in sera were measured by ELISA. For dogs immunized with cNGF-CMV-Ntt830-E4 VLP, anti-NGF-specific IgG antibodies in sera were measured by ELISA.

As described in detail for the immunization with cNGF-CMV-Ntt830-E8* VLPs, Maxisorp ELISA plates were coated with recombinant canine mature NGF protein or CMV-Ntt830-E8*-VLP in 0.1 M Na Carbonate buffer, pH 9.6 at a concentration of 1 μg/ml overnight at 4° C. Plates were washed and SuperBlock™ (PBS) Blocking buffer (Thermo Fisher/Life Technologies Europe) added for 2 hours at RT then washed again. Serum samples were pre-diluted 1:9 or 1:100 in 2% BSA in PBS with 0.05% Tween 20, transferred to the ELISA plates and subjected to ten 3-fold serial dilutions. Following incubation for 2 hours at RT and washing, Horse-radish peroxidase-(HRP-) labelled goat anti-mouse IgG, Fc gamma fragment specific (Jackson ImmunoResearch Europe Ltd) or HRP-labelled rabbit anti-dog IgG (H+L)-HRP, (Jackson ImmunoResearch Europe Ltd) diluted 1:2000 or 1:2500 respectively in 2% BSA in PBS (PBS pH 7.4 (1×) Gibco) with 0.05% Tween-20 was added. After incubation and washing, Pierce™ TMB Substrate Kit (Thermo Fisher/Life Technologies Europe)) was used for colorimetric development. The enzymatic reaction was stopped by the addition of 5% H₂SO₄ and the absorbance at 450 nm measured by spectrophotometry using an ELISA reader (Tecan Spark 10). An OD50 titer describes the reciprocal of the dilution, which reaches half of the maximal OD value.

### Neutralization Assay TF-1 Cells

The neutralizing ability of sera from dogs immunized with cNGF-CMV-Ntt830-E8* VLP and cNGF-CMV-Ntt830-E4 VLP was determined using a bioactivity assay that involved measuring proliferation of the TF-1 erythroblastoma cell line (American Type Culture Collection (ATCC), Manassas, VA).

For the immunization with cNGF-CMV-Ntt830-E8* VLPs, TF-1 cells were harvested, washed three times in PBS (PBS pH 7.4 (1×) Gibco) and cultured overnight in starvation medium (RPMI 1640 Medium (ATCC modification) supplemented with heat inactivated 10% FBS, 1×A/A) at a cell density of 10⁵ cells/ml. 10⁴ TF-1 cells were seeded in a total of 100 μl assay medium (phenol-red free RPMI containing 10% FBS, 2 mM GlutaMax, 10 mM HEPES, 1 mM sodium pyruvate, 4500 mg/L glucose, 1500 mg/L sodium bicarbonate, 100 U/mL Penicillin, 100 μg/mL streptomycin, 25 μg/mL Amphotericin B) per well of a 96-well flat-bottom plate.

To test the in vitro neutralizing activity of antibodies raised by immunization with cNGF-CMV-Ntt830-E8* VLP, sera of immunized dogs were collected and total IgG purified according to manufacturer's instruction using Invitrogen Dynabeads™ Protein G (Thermo Fisher/Life Technologies Europe) for mouse IgG purification and Pierce Protein A Magnetic Beads (Thermo Fisher/Life Technologies Europe) for dog. The capacity of purified total IgGs to neutralize the bioactivity of NGF was tested by incubating a constant concentration of 5 ng/ml human mature NGF (R&D, 256-GF-100/CF) with increasing concentrations of purified dog total IgGs (625-20000 ng/mL), human mature NGF polyclonal antibody (R&D AF-256-NA) or human mature NGF monoclonal antibody (R&D MAB256-500) for 1 hour at room temperature. The NGF-antibody solution was then added to 10⁴ TF-1 cells starved overnight and cell proliferation was quantified over the last 24 hour period of the total 72 hour incubation time using the BrdU based cell Proliferation ELISA (Roche). Manufacturer's instruction were followed and color development was stopped with 5% sulfuric acid. Absorbance was measured at 450 nm with a reference wavelength of 690 nm.

The percent proliferation for each IgG dilution was calculated in relation to the proliferation measured for IgG purified from sera collected at baseline prior to infection (day 0). Data was expressed as percent proliferation versus IgG concentration. GraphPad Prism (version 8.0.0 for Windows, GraphPad Software, San Diego, California USA, www.graphpad.com) was used to fit a sigmoidal 4PL curve to determine the IgG concentration required to achieve 50% inhibition of proliferation (50% Neutralization Titer NT50).

NGF neutralizing antibodies in dogs after the immunization with cNGF-CMV-Ntt830-E4 VLP were determined as follows: TF-1 cells were harvested and washed 3 times with PBS prior to resuspension in starvation medium (Phenol-red free RPMI (Sigma) containing 10% HI-FBS, 2 mM GlutaMax (Gibco), 10 mM HEPES (Sigma), 1 mM sodium pyruvate (Sigma), 4500 mg/L glucose (Gibco), 1500 mg/L sodium bicarbonate, 100 U/mL Penicillin, 100 μg/mL streptomycin, 25 μg/mL Amphotericin B (100× anti-anti Gibco) at a cell density of 2×10⁵ cells/mL. Serum samples were heat inactivated for 30 minutes at 56° C. then diluted 1:25 (4-time final concentration of 1:100) in starvation medium and 2-fold serial dilution was performed. hNGF was diluted to 20 ng/ml (4-times final concentration of 5 ng/mL) and 25 μL added to wells containing 25 μL prediluted serum or 25 μL starvation medium (positive control wells). Instead of hNGF, 50 μL of starvation medium was added to negative control wells. hNGF-serum/antibody mix was incubated for 1 hour at room temperature. Serum starved TF-1 cells were collected, and 50 μL cell suspension were added at a cell density of 1×10⁴ cells/well of a flat bottom 96 well plate. The final sample volume per plate was 100 μL/well. Cell culture plates were incubated for approximately 68 hours at +37° C. in a 5% CO₂ cell culture incubator. Viability of cells was quantitated by the Promega CellTiter 96 Aqueous One Solution Cell Proliferation Assay (Promega). 20 μL of CellTiter 96® Aqueous One Solution Reagent was added per well. Plates were incubated for 7 hours at +37° C. in a humidified, 5% CO₂ incubator. Absorbance at 490 nm with a reference wavelength at 700 nm was recorded. To determine the IC50 values, titration curves were generated by plotting the OD values versus the dilution factor of the serum sample using GraphPad prism software (GraphPad Prism version 8 and 9 for Windows, GraphPad Software, San Diego, California USA). Using a 4-PL regression curve fit model the IC50 values, the dilution factor corresponding to half maximum OD values, were determined. Serum titers of samples at different time points were defined and depicted as the IC50 values of the curve fit.

### Results

### Canine Experiments

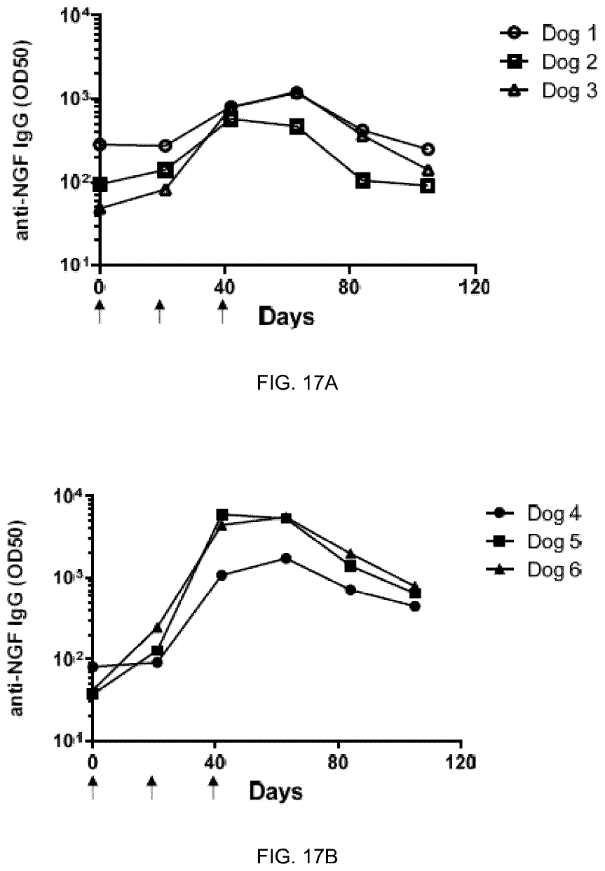

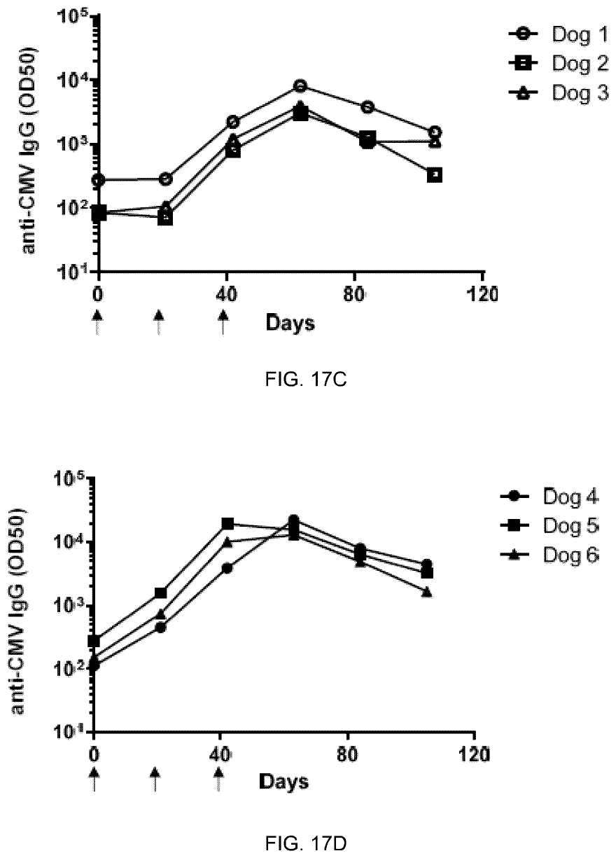

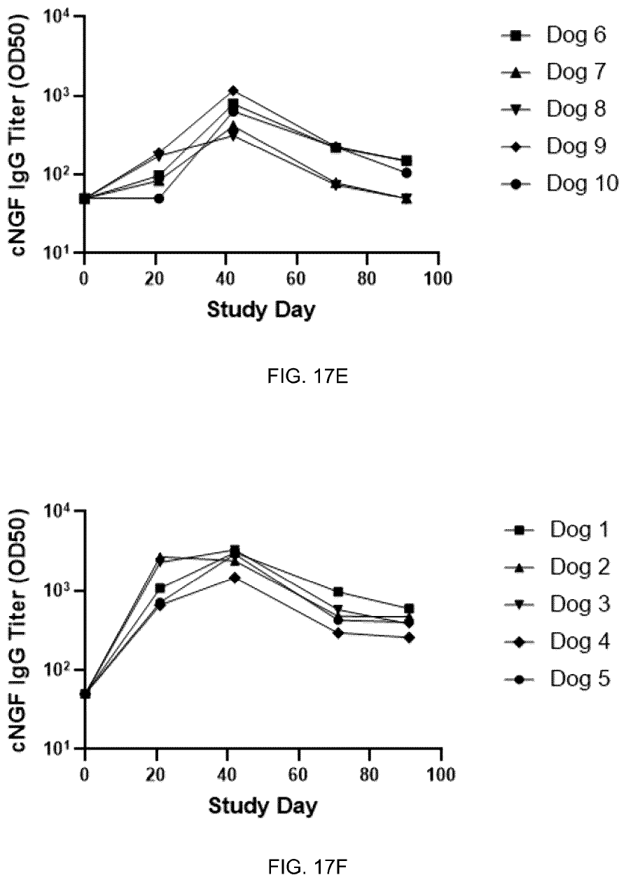

In animals receiving cNGF-CMV-Ntt830-E8* VLPs in the absence of adjuvant, detectable anti-NGF IgG titers were observed in sera collected at day 21 (FIG. 17A) after a single administration of the vaccine. NGF-specific IgG titers were highest in day 42 sera 3 weeks following the second administration of the vaccine. After the third injection on day 42, the titers remained constantly high until day 63 and declined gradually thereafter in all animals. The magnitude of the anti-CMV IgG titers was similar to those measured against canine mature NGF but the kinetic of the response was slightly different (FIG. 17C). The anti-CMV IgG antibodies were somewhat delayed and only unequivocally detectable from day 42 onwards after the second immunization and peak titers were measured in day 63 sera following the third immunization where after the titers declined.

For animals immunized with cNGF-CMV-Ntt830-E8* VLPs in combination with adjuvant Quil A, anti-NGF IgG antibodies were first detected in day 21 sera after a single administration of vaccine on day 0 (FIG. 17B). The second and third doses of vaccine increased the titers in two out of three animals with peak titers measured in sera collected at day 63. The third animal achieved its peak titer at day 42 suggesting the third dose of vaccine may not have increased the antibody response. The kinetics and magnitude of the anti-CMV IgG antibody titers were similar to those measured against canine mature NGF (FIG. 17D).

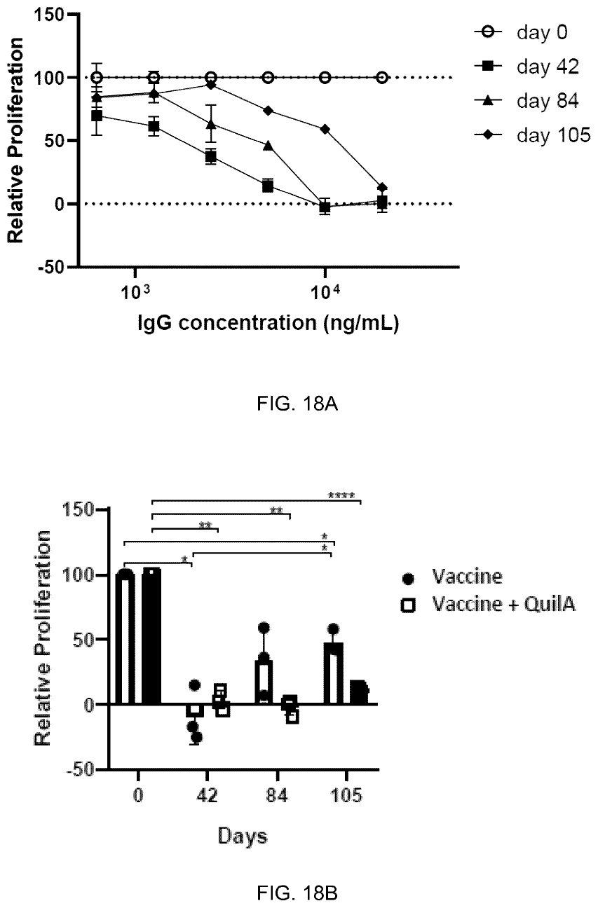

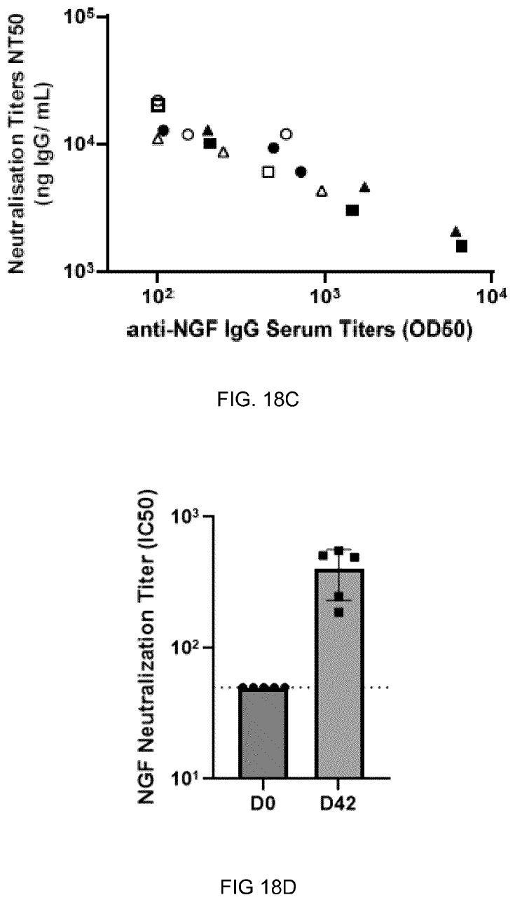

In animals receiving cNGF-CMV-Ntt830-E8* VLPs in the absence of adjuvant, detectable anti-NGF IgG titers were observed in 4 out of 5 study animals in sera collected at day 21 (FIG. 18E) after a single administration of the vaccine. Highest titers were observed 21 days after second dose on day 42.

For animals immunized with cNGF-CMV-Ntt830-E4 VLPs in combination with aluminum hydroxide, anti-NGF IgG antibodies were detected all animals 3 weeks after a single administration of vaccine on day 0 (FIG. 17F). The second dose of vaccine increased the mean group titer.

The neutralizing ability of anti-NGF IgG antibodies induced in response to the vaccination with cNGF-CMV-Ntt830-E8* VLP was analysed using a bioassay based on NGF mediated proliferation of TF-1 cells. IgG antibodies purified from immunized dogs inhibited mature NGF induced proliferation in a concentration dependent manner whereas IgG antibodies purified from pre-immune sera of the same animals failed to do so (FIG. 18A). Vaccination with cNGF-CMV-Ntt830-E8* VLP induced high neutralization titers that could be further increased by co-administration of the vaccine with Quil A adjuvant (FIG. 18B). This observation reflects the anti-NGF ELISA IgG titers in these dogs described above. A clear correlation between the anti-NGF titers and the neutralization capacity was observed (FIG. 18C). IgG purified from sera with high vaccine specific titers had increased potency with respect to inhibition of NGF mediated proliferation of TF-1 cells.

Vaccination with cNGF-CMV-Ntt830-E4 VLP induced neutralizing anti NGF antibody titers. High levels of neutralizing anti-NGF antibodies in the sera collected from dogs immunized twice with cNGF-CMV-Ntt830-E4 in presence of aluminium hydroxide were observed at day 42 (FIG. 18D).

These results show that conjugates of canine mature NGF coupled to modified VLPs comprising chimeric CMV polypeptides in accordance with the present invention are able to overcome immune tolerance to the endogenous target antigen and induce NGF-specific IgG antibodies in dogs, the target species. Moreover, these antibodies were able to efficiently neutralize canine mature NGF activity in vitro.

### Example 7

### Assessment of Efficacy in an Experimental Kaolin Model of Acute Inflammatory Pain in Laboratory Dogs

### Study Objectives

The primary objectives of the study were
(1) to test the efficacy of a preferred composition and vaccine candidate in accordance with the present invention, namely cNGF-CMV-Ntt830-E4 VLP prepared in Example 5, on pain reduction in a kaolin model of inflammatory pain in dogs using positive (non-steroidal anti-inflammatory drug-NSAID) and negative (placebo) controls, and
(2) to compare the efficacy of the inventive composition and vaccine candidate relative to a prior art monoclonal antibody mAb named bedinvetmab (commercialized as Librela®) in the same model.

The secondary objectives of the study were to confirm the kinetics profile of anti-NGF antibodies in serum upon completion of the vaccination phase to ensure that both the inventive vaccine candidate and bedinvetmab efficacies are assessed at the peak of circulating titers for each modalities.

### Study Design:

Twenty-eight (28) dogs were used to investigate the ability of the inventive vaccine to overcome pain associated to inflammation in a kaolin model in laboratory Beagles dogs. Age of dogs at arrival was 8 months. The mean (±SD) body weight was 12.36±1.63 kg. All the dogs were considered healthy based on full clinical examinations performed by a veterinarian and on biochemical and hematological analysis. No animal was removed from the study.

The dogs were allocated to four groups (seven dogs/group): Librela® group (group 1), Vaccine group (group 2), Inflacam® group, a NSAID for dogs (group 3) and Placebo group (group 4).
For the Librela® group, the dogs were treated once subcutaneously at the back of the neck on day 35 (D35) at the dose of 5 mg bedinvetmab/dog for dogs between 5.0 and 10.0 kg and 10 mg bedinvetmab/dog for dogs between 10.1 and 20 kg. The range of the actual doses of bedinvetmab administered was 0.52-0.97 mg/kg in line with the product information of the marketing authorization.
For the Vaccine group, the dogs received the inventive vaccine candidate, cNGF-CMV-Ntt830-E4 VLP, twice subcutaneously at the back of the neck on day 0 (DO) and day 21 (D21) at the dose of 250 μg VLP-NGF/dog. The range of the actual doses of CMV VLP-NGF administered were 0.02-0.03 mg/kg for the first and for the second administrations.
For the Inflacam® group, the dogs received Inflacam® once subcutaneously at the back of the neck on day 43 (D43) at the dose of 0.2 mg meloxicam/kg in line with the product information of the marketing authorization.
For the Placebo group, the dogs received 1 mL of water for injection twice subcutaneously at the back of the neck on DO and D21.

The different dates and regimens of administration of both modalities, i.e. the vaccine candidate, cNGF-CMV-Ntt830-E4 VLP, and the prior art mAb, bedinvetmab, was effected to ensure that their respective peak titers are achieved at the time of the challenge (D42). This means that the CMV VLP-NGF vaccine candidate was given at DO and D21, whereas the mAb was administered at D35 due to its different pharmacokinetics profile.

Blood samples for serology were collected at the following time points on DO, D21, D70, D84, D105 and D119 for the Vaccine and the Placebo groups only, on D35 for the Librela® group only, on D42 and on D56 for the three group. Blood samples were centrifuged and the serum decanted into three aliquots per sample.

Kaolin-induced inflammation in the hind paw was performed based on the model described in “Paw inflammation model in dogs for preclinical pharmacokinetic/pharmacodynamic investigations of nonsteroidal anti-inflammatory drugs” (Jeunesse et al., 2011, J Pharmacol Exp Ther., 338 (2): 548-58). Briefly, the dogs were anaesthetized with an intravenous administration of propofol (Propovet, 6.5 mg/kg, Zoetis, France) and kaolin was injected subcutaneously on one paw. On D42, the injections were performed on the right hind paw of all dogs.

Lameness score was assessed on a typical course made by each dog using a numerical rating scale initially developed by Giraudel et al. (Giraudel J M et al., 2005, Br J Pharmacol 146:642-653; Giraudel J M et al., 2005, J Vet Pharmacol Ther 28:275-285) on cats and adapted by Jeunesse et al. (Jeunesse et al., 2011, J Pharmacol Exp Ther., 338 (2): 548-58). The numerical rating scale for the evaluation of lameness in the inflamed paw was based on the examiner's perception and the score was established as follows: (0) No lameness; (1) Barely detectable lameness over most of the observation period; (2) Mild lameness, substantial weight bearing; (3) Moderate lameness, minimal weight bearing; (4) Severe lameness, the animal uses his paw (walking movement initiated and/or touches lightly the ground) but does not bear weight; (5) The animal could not be more lame, refuse to move and/or avoid any contact of the inflamed paw with the ground. Lameness score was assessed on live and separately on videos by a different operator. Then, the scores were compared and, in case of disagreement, the final say was decided by an operator blinded to the dogs' treatments.

Lameness score was measured on all dogs prior to inflammation induction on D34 and on D36, after inflammation induction one time/day from D43 (one day after kaolin administration) to D52 (10 days after kaolin administration), and from D55 (13 days after kaolin administration) to D56 (14 days after kaolin administration).

### Serology Methods:

### Anti NGF and Anti CMV VLP Total IgG Antibody Responses Measured by ELISA:

NGF- and cNGF-CMV-Ntt830-E4 VLP total specific IgG antibodies in sera were measured by ELISA. ELISA plates were coated with recombinantly produced canine NGF protein (1.0 μg/mL) or cNGF-CMV-Ntt830-E4 VLP (2.0 μg/mL). Serum samples were pre-diluted 1:100, transferred to the ELISA plates and subjected to serial dilutions 3-fold serial dilution steps with a total of 11 dilutions being tested per serum sample. Following incubation, horse-radish peroxidase-(HRP-) labelled goat anti-dog IgG (Jackson ImmunoResearch, cat no 304-035-008, lot 156911) was added. After 2 hours incubation at room temperature, the plates were washed 4 times with 1×PBS/0.05% (v/v) Tween 20. 100 μL per well OPD Substrate prepared according to manufacturer's instruction (OPD tablets (Sigma cat. no. P6912, lot SLBW6989 and SLCJ3691), phosphate citrate buffer tablets (Sigma cat. no. P4809, lot SLCH5937), 30% H2O2 (Sigma cat. No. H1009, lot STBJ6341) was used for colorimetric development. The enzymatic reaction was stopped by addition of 50 μL per well 2 M H₂SO₄ and the absorbance at 490 nm measured by spectrophotometry using an ELISA reader. Titration curves were generated by plotting the OD490 nm values versus the dilution factor of the serum sample. Using a 4-Parameter logistic regression curve fit model the EC50 values, the dilution factor corresponding to half maximum OD490 nm values, were determined. Serum titers of samples at different time points were defined and depicted as the EC50 values of the curve fit. Titers below the detection limit of the assay were set to 100 for anti-CMV IgG respectively 50 for anti-NGF IgG, half of the lowest dilution factor (1:200 respectively 1:100) used in the assay. Bedinvetmab was used as a standard in the NGF IgG ELISA. Relative concentrations of anti-NGF IgG antibody in the serum (μg/mL) were calculated by multiplying the EC50 values of the serum with the EC50 values determine for the Bedinvetmab standard. Neutralization titers below the detection limit of the assay were set the equivalent monoclonal antibody concentration of the EC50 value of 50.

### Ngf Neutralizing Antibodies Titers Measured by a Non-Cell-Based Assay:

ELISA plates were coated with 100 μL/well of 0.5 μg/mL TrkA (SinoBio 11073-H08H) in PBS over night at +4° C. After washing with PBST, plates were blocked using 200 μL/well SuperBlock (ThermoScientific 37518) for 1 hour at room temperature. Serum samples or positive control antibody (caninized monoclonal anti-canine NGF antibody bedinvetmab, Zoetis) were diluted in 2% BSA/PBST and incubated with 1.25 ng/ml hNGF (R&D 256-GF) for 1 hour. After washing the ELISA plates, serum/NGF solution was added to the plates. Plates were incubated for 1 hour at room temperature, washed and HRP-labelled rabbit anti-NGF antibody (SinoBio 11050-RP02-H) at a concentration of 1.5 μg/mL was added. After the plates were washed 4 times with 1×PBS/0.05% (v/v) Tween 20, TMB substrate (ThermoScientific 3401) as the HRP substrate and development reaction was stopped by addition of 2M H₂SO₄. OD values at 450 nm were measured using a microplate reader (TecanSpark). Titration curves were generated by plotting the OD 450 nm values versus the dilution factor of the serum sample. Using a 4-Parameter logistic regression curve fit model the IC50 values, the dilution factor corresponding to half maximum OD 450 nm values, were determined. Serum titers of samples at different time points were defined and depicted as the IC50 values of the curve fit. Neutralization titers below the detection limit of the assay were set to 50, corresponding to half of the lowest dilution factor (1:100) used in the assay. Relative concentrations of neutralizing antibody in the serum (μg/mL) were calculated by multiplying the IC50 values of the serum with the IC50 values determine for the bedinvetmab standard (μg/mL). Neutralization titers below the detection limit of the assay were set to the equivalent monoclonal antibody concentration of the IC50 value of 50.

### Ngf Neutralizing Antibodies Titers Measured by a TF-1 Cell Based Assay:

Heat-inactivated (56° C. 30 min), serial-diluted sera or antibody standard (canonized monoclonal anti-canine NGF antibody bedinvetmab, Zoetis) were incubated with 5 ng/mL human beta-NGF for 1 hour at room temperature. The NGF-sera/antibody solution was then added to 104 TF-1 cells starved overnight of growth factors. Cell viability was quantified over the last 7 hour period of a total 72 hour incubation period using the CellTiter 96 AQueous One Solution Cell Proliferation Assay reagent (Promega). Absorbance was measured using a microplate reader (TecanSpark) at a wavelength of 490 nm and a reference wavelength of 700 nm. Titration curves were generated by plotting the difference in absorbance (490 nm-700 nm) versus the dilution factor of the serum sample. Using a 4-Parameter logistic regression curve fit model the IC50 values, the dilution factor corresponding to half maximum OD values, were determined. Serum titers of samples at different time points were defined and depicted as the IC50 values of the curve fit. Neutralization titers below the detection limit of the assay were set to 50, the half of the lowest dilution factor (1:100) used in the assay. Titers above detection limit of assay but too low to reliably determine neutralization titers were set to 100. Relative concentrations of neutralizing antibody in the serum (μg/mL) were calculated by multiplying the IC50 values of the serum with the IC50 values determine for the bedinvetmab standard. Neutralization titers below the detection limit of the assay were set to the equivalent monoclonal antibody concentration of the IC50 value of 50. Titers above detection limit of assay but too low to reliably determine neutralization titer were set to the equivalent monoclonal antibody concentration of the IC50 value of 100. NGF IgG antibodies in dog sera are presented as EC50 titers or as antibody standard equivalent titers.

### Serology Results:

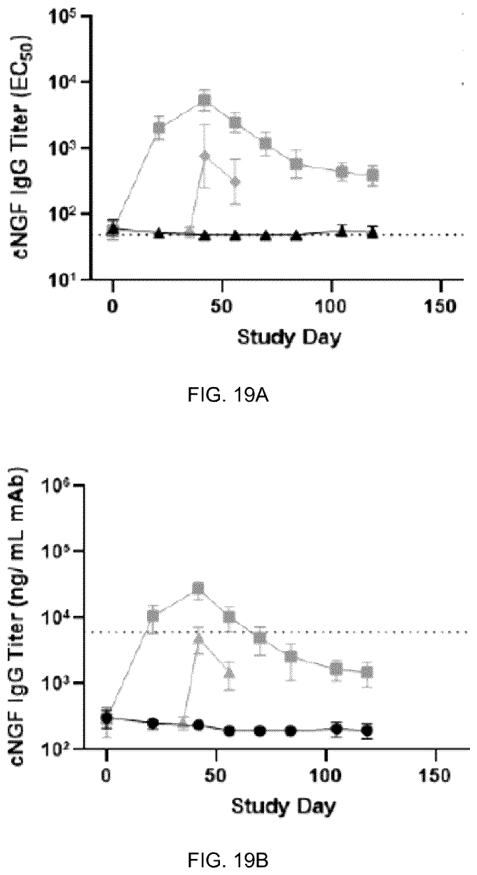

In group 2 animals treated with cNGF-CMV-Ntt830-E4 VLP, NGF binding IgG antibodies were detectable 3 weeks after the first dose and increased in response to the second administration on study day 21. All animals in the group responded to treatment. Peak titers were observed on day 42 with GMTs of 25.6 μg/mL (95% CI: 36.7, 17.9). Day 42 was also the time point selected for injection of kaolin into the footpad. The antibody response declined in two phases. First a rapid decline phase with a relative short antibody half-life between day 42 and day 84 followed by a persistent more stable phase with a longer antibody half-life observed between study days 84 and 119. The bi-phasic kinetic observed for the NGF IgG response is typical for antibody responses after vaccination. The NGF-specific IgG GMTs in group 1, (bedinvetmab treated group), also reached its peak value at day 42, i.e. 7 days after injection and day of 1st kaolin injection (3.68 μg/mL; (95% CI: 11.3, 1.197) which value, however, was significantly lower than the corresponding anti-NGF-specific antibody titers induced in the Vaccine-treated group (group 2) that received the inventive vaccine (FIG. 19A and FIG. 19B).

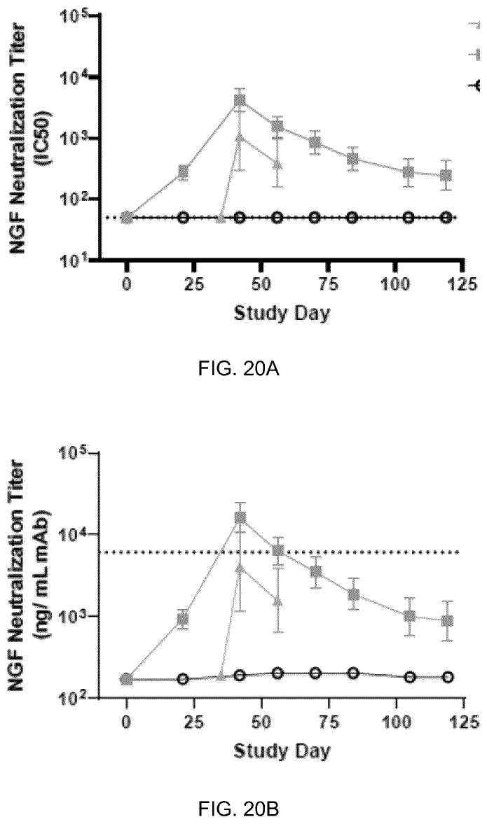

Overall, the kinetic of the NGF neutralizing antibodies determined by the TrkA based NGF binding ELISA, was comparable to the kinetic profile of NGF binding IgGs. Neutralizing antibodies were observed in 7 out of 7 dogs 21 days after first dosing of the CMV VLP-NGF vaccine and remained above the detection level of the assay for all animals in this group. Comparable to the NGF binding IgG titers, peak NGF neutralizing titers were measured 21 days after the 2nd dose on day 42 (16.0 μg/mL (95% CI: 24.0, 10.6)) and waned thereafter. The CMV VLP-NGF vaccine-induced standard equivalent titers exceeded or were at comparable levels of peak serum titers observed 7 days after bedinvetmab injection (FIG. 20A and FIG. 20B).

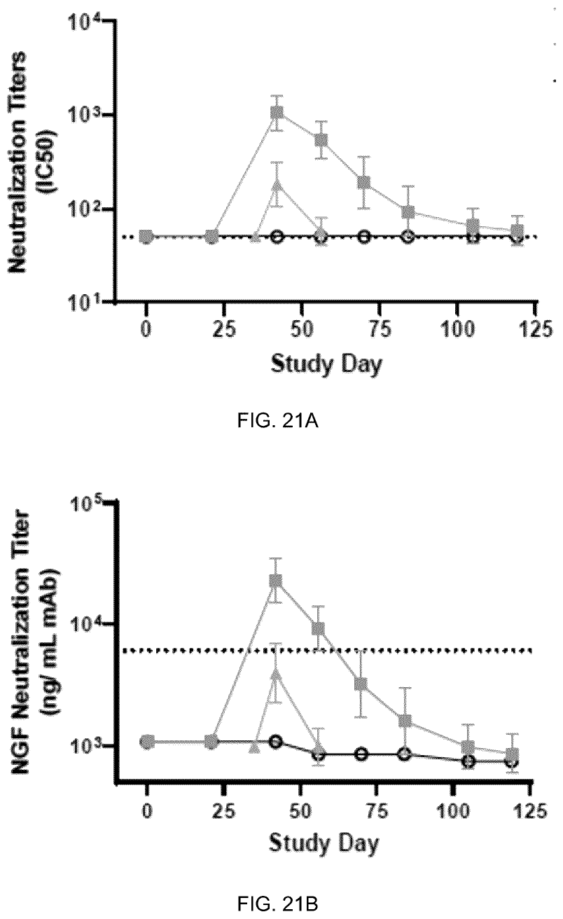

Overall, neutralization titers measured by the TF-1 cell based bioassay were similar to the titers determined by the TrkA-based NGF binding ELISA. Although the threshold level to detect neutralizing antibodies was slightly higher for the cell-based assay, higher titers were measured with this assay once this threshold level was exceeded. Due to the higher threshold level to detect NGF neutralizing antibodies in the TF-1 cell-based assay, neutralizing antibodies in group 2 were not detected on day 21. However, they were detected in 7 out of 7 animals 21 days after the second VLP-NGF administration (FIG. 21A and FIG. 21B).

In dogs receiving bedinvetmab (study group 1), peak titers of 3.93 μg/mL (95% CI: 6.90, 2.24) were observed on study day 42, the day of the first kaolin challenge. NGF neutralizing antibodies were not observed in one dog at any other time point analyzed. 21 days after administration on study day 56, the bedinvetmab levels were below the detection limit of the TF-1-based NGF neutralization assay in 6 of 7 dogs.

In summary, the CMV VLP-NGF vaccine induced higher neutralizing anti-NGF antibody titers in the serum compared to the neutralizing anti-NGF antibody titers that are obtainable by the administration of bedinvetmab according to the marketing authorization.

### Pharmacodynamic Results (Alleviation of Pain in the Kaolin Model):

AUC (Area Under the Curve) of the pharmacodynamic endpoint measurements were thus compared over time between the different treatment groups. Mean lameness score before and for each day after kaolin-induced inflammation. No dog presented lameness before kaolin-induced inflammation. Statistical analysis (t-test for data with normal distribution and Mann-Whitney (non-parametric) otherwise) were performed using RStudio (R 4.1.0, R Development Core Team, R Foundation for Statistical Computing, Vienna, Austria) to test the significance of the difference between the AUC for each endpoint for the Vaccine group treated with the CMV VLP-NGF vaccine candidate and the Placebo (water for injection) groups. The bedinvetmab and the INFLACAM® groups were analyzed and compared in a descriptive way.

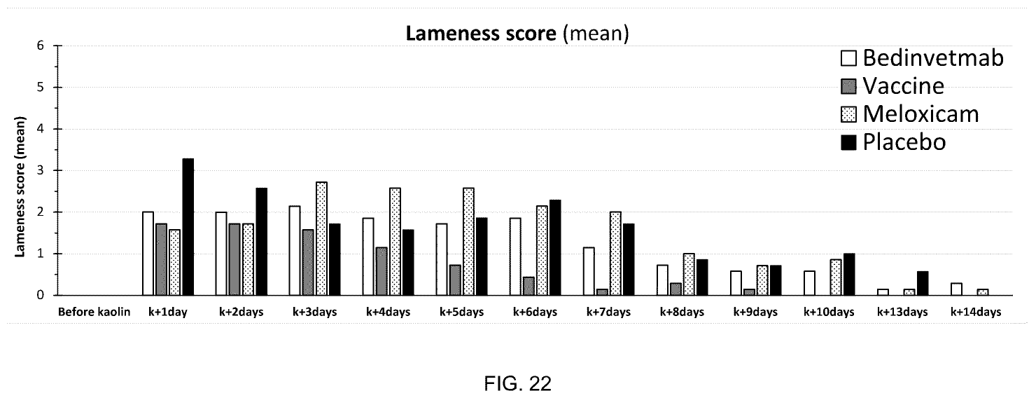

Inflacam® group: One day after kaolin-induced inflammation, the mean lameness score reduced for the Inflacam® group as compared to the Placebo group (FIG. 22). Then the lameness score increased for the Inflacam® group and the evolution over time became similar to that of the negative control group with a resolution of the lameness from 10 days post-inflammation. These results validate the inflammation model with meloxicam as a positive control (FIG. 22). The fact that the lameness score was similar in both groups as soon as 3 days after kaolin-induced inflammation is explained by the duration of the NSAID's action that does not exceed 48 hours.

For the Vaccine group (CMV VLP-NGF vaccine), some lameness was detectable until day 3 after kaolin-induced inflammation but significantly dropped over the detection period. As early as 6 days post-inflammation, no lameness was observed in the Vaccine group for the CMV VLP-NGF vaccine (FIG. 22).

The lameness was more marked in the bedinvetmab group than in the Vaccine group. Starting from day 3 after kaolin-induced inflammation, mean lameness scores for the CMV VLP-NGF vaccine were significantly lower as compared to the bedinvetmab group over the entire detection period until day 14 (FIG. 22).

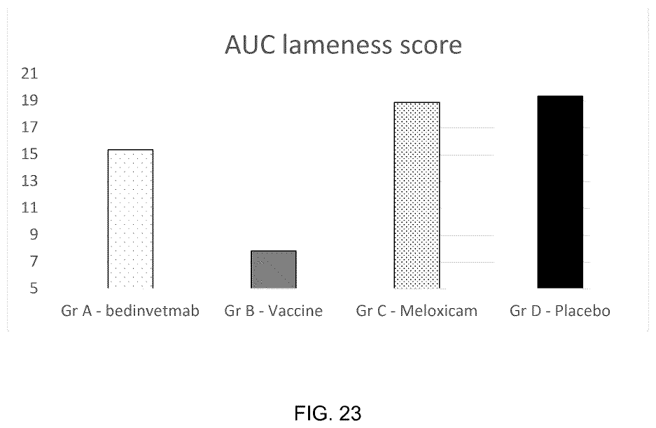

The mean AUCs for lameness scores over the first period were 15.36, 7.80, 18.87 and 19.34 for the bedinvetmab, the CMV VLP-NGF vaccine, the Inflacam® and the Placebo groups, respectively. A significant treatment effect in the lameness model was reported between the Vaccine and the Placebo groups. Moreover, there was significant improvement of the AUCs lameness scores between the CM VLP-NGF vaccine and the bedinvetmab treatment group (FIG. 23), which both underlines the high efficacy of the CMV VLP-NGF vaccine on all the dogs over the whole period. Interesting to note that the AUC lameness score was much more variable in the bedinvetmab, the Inflacam® and the Placebo groups than in the CMV VLP-NGF vaccine group, which is further evidence of the excellent efficacy of the NGF vaccine (data not shown).

### Example 8

### Assessment of Efficacy in a Surgical Experimental Model of Chronic Pain in Laboratory Dogs

### Study Objectives

Objective of this study was to evaluate the efficacy of the CMV VLP-NGF vaccine to relieve osteoarthritis (OA) pain in a canine Anterior Cruciate Ligament Transection and Partial Medial Meniscectomy (ACLT+PMM) model.

The use of slowly progressive experimental models of pain has revealed that distinct pain mechanisms operate in a time-dependent manner. Because of the chronic progressive nature of OA in dogs, it is expected that mechanisms of pain could differ in early vs. late stages of this disease in dogs. It is therefore important to confirm the pharmacodynamic of the CMV VLP-NGF vaccine under conditions that model appropriately the chronic phase of the disease. This is all-the-more-so important since there is evidence that NGF plays a role in nociception related to non-inflammatory pain.

Based on prior evidence with a destabilization of medial meniscus (DMM) experimental model in rodents, there is a rationale to use a surgical model leading to the stifle joint destabilization to mimic initial inflammatory pain and its subsequently evolution into non-inflammatory chronic pain in dogs under experimental conditions.

In parallel to the longitudinal clinical assessment of chronic pain in dogs upon anterior cruciate ligament transection and partial medial meniscectomy (ACLT+PMM), longitudinal serological testing was implemented to investigate the correlation between circulating titers of anti NGF antibodies and pharmacodynamic assessments.

Serological investigations were performed using the exact same analytical methods previously described in Example 7 for the assessment of efficacy in an experimental kaolin model of acute inflammatory pain in laboratory dogs.

### Study Design:

24 adult (older than 12 months at study inclusion) beagle dogs were allocated into 3 study groups. Dogs in study group A were treated with the CMV VLP-NGF vaccine (cNGF-CMV-Ntt830-E4 VLP at a dose of 250 μg/dose), animals in study group B were treated with Librela® (bedinvetmab, benchmark product) and dogs in study group C were left untreated. Librela® (Zoetis), is a commercially available recombinantly expressed canine monoclonal antibody (mAb) against NGF (bedinvetmab) registered for the treatment of pain associated with osteoarthritis in dogs and was administered subcutaneously monthly according to the manufacturer's instructions from the day of surgery onwards. The CMV VLP-NGF vaccine was administered subcutaneously on four occasions on study days 0, 21, 91, and 203. From study day 42 to 47, all animals included in the study underwent an anterior cruciate ligament resection and a partial meniscectomy on their right hind knee. Pain scoring and clinical lameness evaluation were performed 12 days after surgery (study day (SD) 54-59) and on day 84, day 133, day 182, day 203, and day 217. Serology was performed for animals treated with cNGF-CMV-Ntt830-E4 VLP of group A (CMV VLP-NGF vaccine). Serology was also performed for animals in group B (bedinvetmab), on samples from study day 203 to confirm presence of anti-NGF antibodies.

### Serology Methods:

Serology methods used in this study are identical to the ones previously described for the kaolin model in laboratory dogs in Example 7.

### Serology Results:

### Anti NGF and Anti CMV VLP Total IgG Antibody Responses Measured by ELISA:

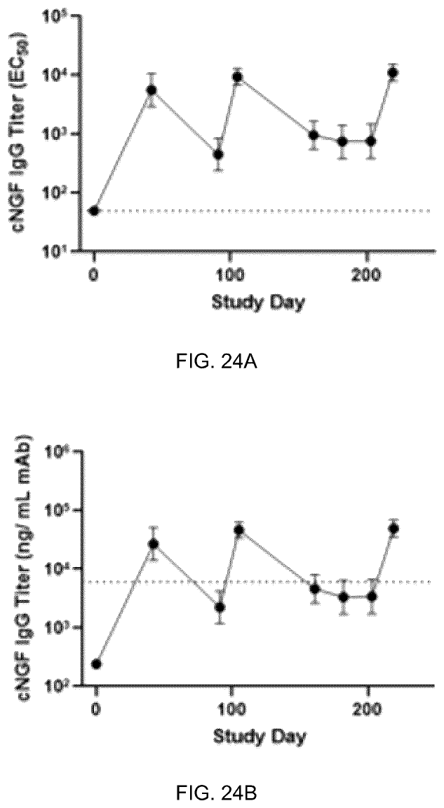

In group A (CMV VLP-NGF vaccine), all animals responded to the CMV VLP-NGF treatment (FIG. 24A and FIG. 24B). Peak titers were observed 2 to 3 weeks after second, third and fourth dose. In the week of the surgery on day 42, GMTs were 27.2 μg/mL (95% CI: 51.9, 14.2). As expected, NGF-specific IgG antibodies waned thereafter descending to levels of 2.25 μg/mL (95% CI: 4.25, 1.19) by the day of the third dose, study day 91. At this time point, in one animal NGF IgG titers remained above or near the peak mean NGF GMTs reported for bedinvetmab (i.e., 6.1 μg/mL) After the boost in titers measured on day 105 resulting from the third injection of CMV VLP-NGF vaccine, the antibody response declined in two phases. A rapid decline phase with a relatively short antibody half-life between day 105 and day 161 (t½ 17 days) was followed by a persistent more stable phase with a longer antibody half-life observed between study days 161 and 203 (t½ 96 days). The bi-phasic kinetic observed for the NGF IgG response after CMV VLP-NGF treatment is typical for antibody responses after vaccination. Importantly, 3 months after the third injection applied on day 91, on day 203, antibody titers remained above or near peak levels reported for bedinvetmab (i.e., 6.1 μg/mL) with a monthly administration. However, antibody titers were increased above the level of 6.1 μg/mL (bedinvetmab treatment) in response to the fourth vaccination. The NGF-specific IgG GMT measured in day 203 sera from group B animals (bedinvetmab treatment), was 10.9 μg/mL (95% CI: 14.9, 8.02); slightly above the expected range (data not shown). All animals in this group were responders and had mean titers of 1 μg/mL (dashed line FIG. 24B).

### Ngf Neutralizing Antibodies Titers Measured by a Non-Cell-Based Assay:

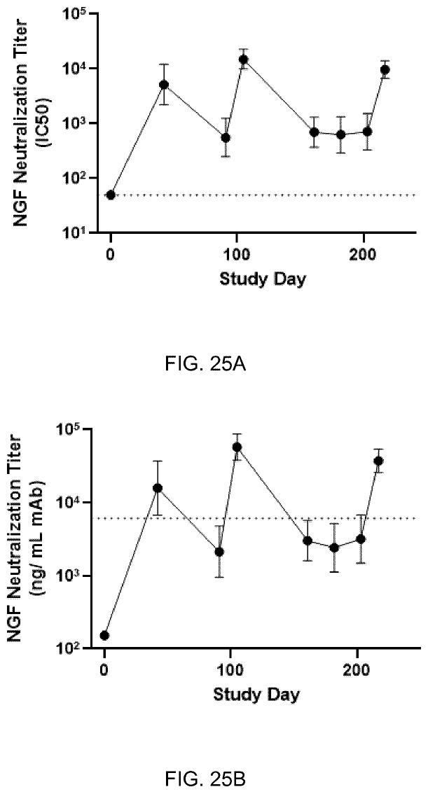

Overall, the kinetic of the NGF neutralizing antibodies determined by the TrkA-based NGF binding ELISA was comparable to the kinetic profile of total NGF-binding IgGs measured by ELISA. On day 42, neutralizing antibodies were observed in all 8 dogs administered the CMV VLP-NGF vaccine. All animals remained above the detection level of the assay throughout the study (FIG. 25A and FIG. 25B). Comparable to the NGF-binding IgG titers, peak NGF neutralizing titers were measured after 2nd, 3rd and 4th dose on day 42 (15.9 μg/mL (95% CI: 37.3, 6.78), day 105 (57.6 μg/mL (95% CI: 87.7, 37.9)) and day 217 (37.6 μg/mL (95% CI: 54.5, 25.9). On day 203, NGF neutralizing antibody titers in the bedinvetmab treatment group B were 7.85 μg/mL (95% CI: 10.4, 5.95) and herewith in the expected range 6 days after bedinvetmab administration (data not shown). Furthermore, co-administration of NSAID together with the CMV VLP-NGF vaccine after the priming phase did not influence the maintenance and boosting of the NGF antibody titers (data not shown).

### Ngf Neutralizing Antibodies Titers Measured by a TF-1 Cell Based Assay:

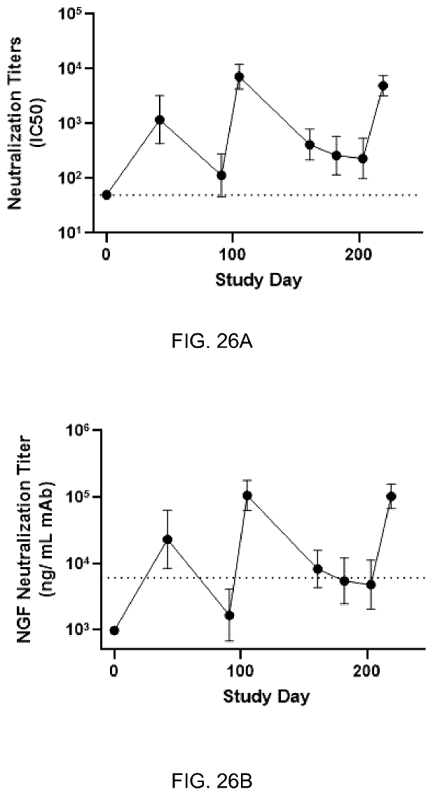

Overall, the neutralization titers measured by the TF-1 cell-based bioassay were similar to those determined by the TrkA-based NGF binding ELISA (FIG. 26A and FIG. 26B). Although the threshold level to detect neutralizing antibodies was slightly higher for the cell-based assay, higher titers were measured with this assay once this threshold level was exceeded. Neutralizing antibodies were induced by the CMV VLP-NGF vaccine and detectable over the entire study period. Significant higher peak levels of neutralizing antibodies could be induced by the CMV VLP NGF vaccine as compared to the bedinvetmab treatment, which had mean titers of 6.1 μg/mL (dashed line FIG. 26B).

### Surgical Methods

Surgery was conducted from D42 onward until D47, with animals of randomized group origin per surgery day (at least one animal per group per surgery day) operated daily. The surgeon(s) was blinded before and during surgery regarding the groups the animals had been allocated to. Under general anaesthesia, a standard mini-arthrotomy was performed at the medial side of the right knee. A transection of the craniomedial part of the anterior cruciate ligament (ACL) at its approximate mid-point was performed using a surgical blade (N°11). A partial medial meniscectomy was performed using a rongeur. Then the joint was thoroughly rinsed with sterile NaCl 0.9% and the capsula was closed using a simple interrupted pattern with a monofilament 2-0. Skin and subcutaneous tissue were closed in single layers.

### Pharmacodynamic Results (Alleviation of Pain in the Surgical Model)

Significant differences were observed in the pain and lameness assessment between the groups.

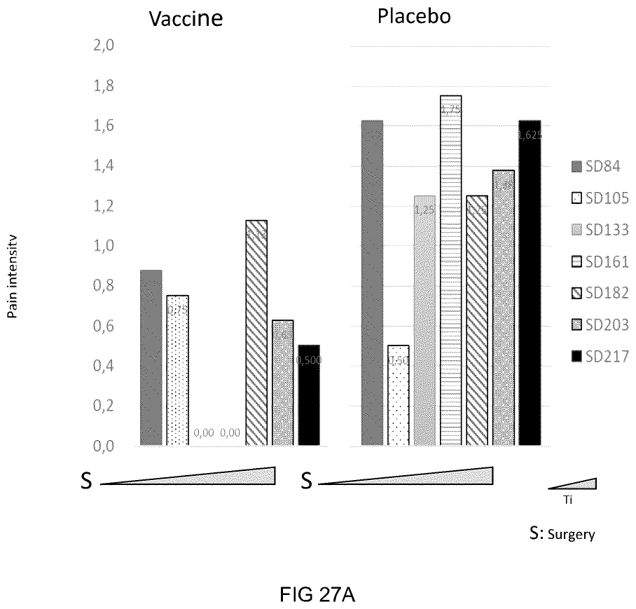

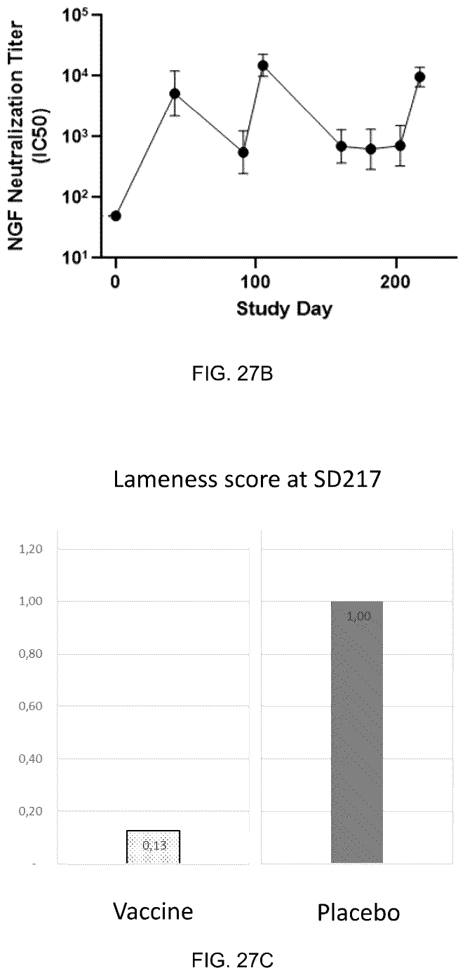

The pain level was measured by palpation of surgically-disabled stifle joints using a semi-quantitative point scoring of mechanical allodynia. The significant level of pain observed shortly post-surgery at D84 is considered related to the inflammation associated to the procedure. This level of pain was transient and waned to baseline level at D105 (FIG. 27A). Subsequently, as the damages to the joint structure evolved as a consequence of surgically-induced biomechanical instability, chronic pain evolved from D133 onwards in the non-treated control group C leveling off on average scores between 1.25 and 1.75. A significant reduction of chronic pain even below detection level was observed at both D133 and D161 in the vaccinated group A, both timepoints corresponding to high level of anti-NGF circulating antibodies (FIG. 27B). At timepoints D182 and D203, (after 91 and 113 days after the third vaccine injection) vaccinated dogs developed a certain degree of pain, however, on a significant lower level than the non-vaccinated placebo group C. Development of some pain in the vaccinated group A was expected according to the vaccination scheme, as anti NGF circulating antibodies had slowly waned at these timepoints. Upon the fourth injection of the vaccine at D217, anti NGF circulating antibodies were significantly boosted again (FIG. 27B) (to levels not achievable with the bedinvetmab treatment), which correlated to significant reduction of the pain level measured in the CMV VLP-NGF vaccinated group A vs. the control group C.

This data demonstrates that the inventive NGF VLP vaccine not only provides a significant reduction of pain in an acute OA pain model, but also provides significant reduction of pain in a chronic settings. It also illustrates the correlation between anti NGF-induced antibody titers and pain reduction, which provides clear evidence for the vaccine induced anti-NGF-self-antibody-mediated mechanism of action. In addition, at D217 upon full development of intra-articular lesions associated with the model, evaluation of lameness at walk and at trot provided clear evidence of reduced pain in the CMV VLP-NGF vaccine group A as compared to the untreated control group C (FIG. 27C).

Collectively, both data (pain and lameness scores) supporting an alleviation of pain upon palpation in the phase of chronic pain establishment post-surgery and data indicating reduced lameness at D217 demonstrate the ability for the CMV VLP-NGF vaccine to control effectively chronic pain related to osteoarthritis in dogs.

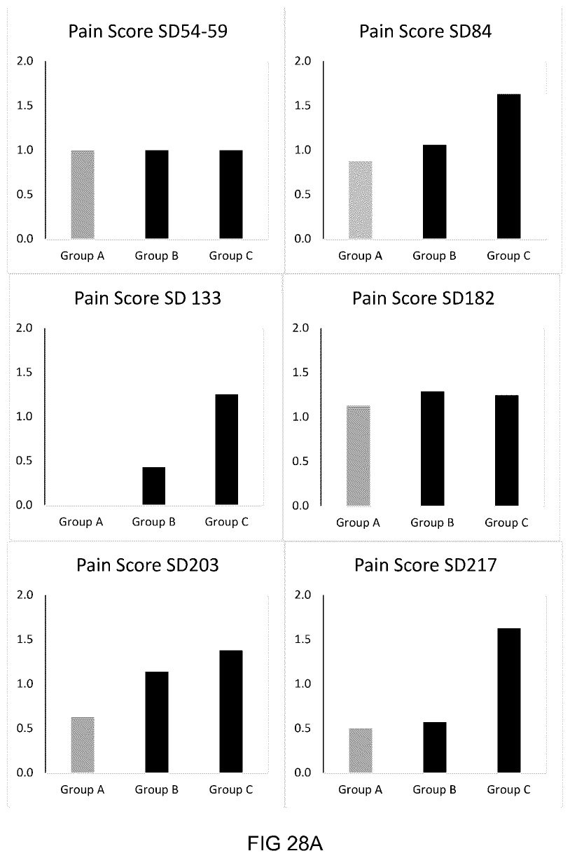

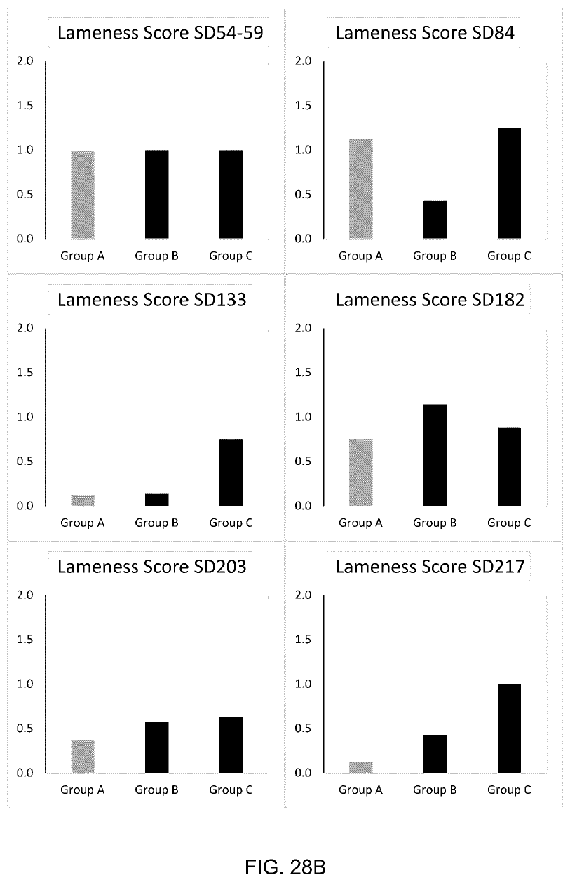

When compared pain and lameness scores to the bedinvetmab treatment group B, lower pain and lameness scores were obtained for the CMV VLP-NGF starting from study day 133 until the end of the study at day 207, clearly supporting the beneficial therapeutic effect of the CMV VLP-NGF vaccine vs. the bedinvetmab treatment (FIG. 28A and FIG. 28B).

1. A method of treating a NGF-related disorder in canine comprising the administration of a composition to said canine, wherein the composition comprises
(a) a virus-like particle (VLP) of Cucumber Mosaic Virus (CMV), wherein said CMV VLP comprises at least one first attachment site; and
(b) at least one antigen, wherein said antigen comprises at least one second attachment site, and wherein said antigen is a nerve growth factor (NGF) antigen; and
wherein (a) and (b) are linked through said at least one first and said at least one second attachment site.

2. The method according to claim 1, wherein the NGF-related disorder is pain.

3. The method according to claim 2, wherein said pain is selected from the group consisting of nociceptive pain, inflammatory-related pain, postsurgical pain, pain associated with musculoskeletal diseases, pain associated with degenerative joint disease and/or osteoarthritis (OA)-associated pain.

4. The method according to claim 2, wherein said pain is pain associated with degenerative joint disease.

5. The method according to claim 2, wherein said pain is acute pain associated with degenerative joint disease.

6. The method according to claim 2, wherein said pain is chronic pain associated with degenerative joint disease.

7. The method according to claim 2, wherein said pain is refractory pain associated with degenerative joint disease.

8. The method according to claim 6, wherein said pain is chronic, refractory pain associated with degenerative joint disease.

9. The method according to claim 1, wherein the composition is administered to said canine in one or several doses.

10. The method according to claim 9, wherein the composition is administered to said canine in at least two doses wherein the time interval between the first and the second dose is at least 7 days.

11. The method according to claim 9, wherein the composition is administered to said canine in at least two doses, wherein time interval between the first and the second dose is between 7 and 21 days.

12. The method according to claim 9, wherein the composition is administered to said canine in at least three doses, wherein time interval between the first and second dose is between one to three weeks, the time interval between the second and third dose is between two to six months, and the time interval between any further dose to the previous dose is between three to six months.

13. The method according to claim 1, wherein the composition is administered to said canine in an amount of 50 to 300 μg/dose.

14. The method according to claim 1, wherein the composition is administered to said canine subcutaneously, intramuscularly or transdermal.

15. The method according to claim 1, wherein (a) and (b) are linked through said at least one first and said at least one second attachment site via at least one covalent non-peptide bond.

16. The method according to claim 1, wherein the VLP comprises an antigenic VLP fusion polypeptide, and wherein (a) and (b) are linked through said at least one first and said at least one second attachment site via at least one covalent peptide bond by the way of fusion.

17. The method according to claim 1, wherein said CMV VLP comprising the at least one first attachment site comprises a CMV polypeptide, wherein said CMV polypeptide comprises a coat protein of CMV or an amino acid sequence having a sequence identity of at least 75% with SEQ ID NO:39.

18. The method according to claim 1, wherein said composition comprises
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises
(i) a CMV polypeptide, wherein said CMV polypeptide comprises a coat protein of CMV or an amino acid sequence having a sequence identity of at least 75% with SEQ ID NO:39; and
(ii) a polypeptide comprising a stretch of consecutive negative amino acids, wherein said negative amino acids are independently selected from aspartic acid or glutamic acid;
(b) at least one antigen, wherein said antigen comprises at least one second attachment site, and wherein said antigen is a nerve growth factor (NGF) antigen; and
wherein (a) and (b) are linked through said at least one first and said at least one second attachment site.

19. The method according to claim 1, wherein the CMV VLP further comprising a T helper cell epitope.

20. The method according to claim 19, wherein the T helper cell epitope is derived from tetanus toxin or is a PADRE sequence.

21. The method according to claim 19 and wherein the T helper cell epitope replaces a N-terminal region of said CMV polypeptide, wherein said N-terminal region of said CMV polypeptide corresponds to amino acids 2-12 of SEQ ID NO:39.

22. The method according to claim 18, wherein said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of:
(a.) a polyglycine linker (G-linker) having an amino acid sequence (Gly)ₙ of a length of n=2-10;
(b.) a glycine-serine linker (GS-linker) comprising at least one glycine and at least one serine; and
(c.) an amino acid linker (GS*-linker) comprising at least one Gly, at least one Ser, and at least one amino acid selected from Thr, Ala, Lys, and Cys.

23. The method according to claim 22, wherein said stretch of consecutive negative amino acids consists solely of glutamic acids.

24. The method according to claim 23, wherein said polypeptide comprising the stretch of consecutive negative amino acids consists of SEQ ID NO:49, SEQ ID NO:50 or SEQ ID NO:51.

25. The method according to claim 1, wherein said CMV VLP comprises the amino acid sequence of SEQ ID NO:10, SEQ ID NO:11 or SEQ ID NO:12.

26. The method according to claim 1, wherein said NGF antigen is selected from the group consisting of canine NGF (cNGF), feline NGF (fNGF), equine NGF (eNGF), bovine NGF (bNGF) and porcine NGF (pNGF).

27. The method according to claim 1, wherein said NGF antigen comprises an amino acid sequence selected from any of SEQ ID NO:30, SEQ ID NO:31, SEQ ID NO:33 and SEQ ID NO:55, or an amino acid sequence having a sequence identity of at least 90% with any of SEQ ID NO:30, SEQ ID NO:31, SEQ ID NO:33 and SEQ ID NO:55.

28. The method according to claim 2, wherein said pain is acute OA associated pain.

29. The method according to claim 2, wherein said pain is chronic OA associated pain.


SEQUENCE LISTING (58 sequences)

SEQ ID NO: 1 - protein, 4 aa, synthetic construct
EEEE

SEQ ID NO: 2 - protein, 8 aa, synthetic construct
EEEEEEEE

SEQ ID NO: 3 - protein, 12 aa, synthetic construct
EEEEEEEEEEEE

SEQ ID NO: 4 - protein, 7 aa, synthetic construct
CGGGSGS

SEQ ID NO: 5 - protein, 222 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 6 - protein, 14 aa, synthetic construct
QYIKANSKFIGITE

SEQ ID NO: 7 - DNA, 672 bp, synthetic construct
  source 1..672: mol_type = other DNA
atgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtgggtcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataa

SEQ ID NO: 8 - protein, 4 aa, synthetic construct
GGGS

SEQ ID NO: 9 - protein, 6 aa, synthetic construct
GGGSGS

SEQ ID NO: 10 - protein, 236 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 11 - protein, 240 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 12 - protein, 241 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEECGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 13 - protein, 244 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 14 - DNA, 720 bp, synthetic construct
  source 1..720: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 15 - DNA, 732 bp, synthetic construct
  source 1..732: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 16 - DNA, 736 bp, synthetic construct
  source 1..736: mol_type = other DNA
catatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagaggaagaagagtgcgggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 17 - DNA, 744 bp, synthetic construct
  source 1..744: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaagaggaagaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 18 - DNA, 51 bp, synthetic construct
  source 1..51: mol_type = other DNA
tgcgggggtggttcgggttcttattatggtaaaaggttgttattacctgat

SEQ ID NO: 19 - DNA, 62 bp, synthetic construct
  source 1..62: mol_type = other DNA
tggatccggtggcggtagcgaagaggaagaagaggaagaagagtgcgggggtggttcgggtt

SEQ ID NO: 20 - DNA, 37 bp, synthetic construct
  source 1..37: mol_type = other DNA
caaagcttatcaaactgggagcaccccagatgtggga

SEQ ID NO: 21 - DNA, 24 bp, synthetic construct
  source 1..24: mol_type = other DNA
aggatcgagatctcgatcccgcga

SEQ ID NO: 22 - DNA, 35 bp, synthetic construct
  source 1..35: mol_type = other DNA
accataataggatccacggtctatttttggtggct

SEQ ID NO: 23 - DNA, 40 bp, synthetic construct
  source 1..40: mol_type = other DNA
agaccgtggatcctattatggtaaaaggttgttattacct

SEQ ID NO: 24 - DNA, 25 bp, synthetic construct
  source 1..25: mol_type = other DNA
agtaccggtgaggctccgtccgcaa

SEQ ID NO: 25 - DNA, 51 bp, synthetic construct
  source 1..51: mol_type = other DNA
ggatccggtggcggtagcgaagaggaagaagggggtggttcgggttcttat

SEQ ID NO: 26 - DNA, 63 bp, synthetic construct
  source 1..63: mol_type = other DNA
ggatccggtggcggtagcgaggaagaggaagaagaggaagaagggggtggttcgggttcttat

SEQ ID NO: 27 - DNA, 75 bp, synthetic construct
  source 1..75: mol_type = other DNA
ggatccggtggcggtagcgaggaagaagaggaagaagaggaagaagaggaagaagggggtggttcgggttcttat

SEQ ID NO: 28 - DNA, 698 bp, synthetic construct
  source 1..698: mol_type = other DNA
ccatggccgaacctcacccagagtcaaacgttcccgcgggacatgcgattccacaggcgcactggacaaaacttcaacattcgttagacacagctcttcgccgtgcccgcagcacccctgcgggcgcaatcgcagcccgtgtagcgggtcaaacccgtaatattacagtggatccgaaattgtttaaaaagcgccgtctgcgtagtccccgcgtgttattctccacgcatcccccccctgttgccgcggatacccaaggcttagatctggaagctgggggagctgcatctttcaaccgcacgcaccgctcgaaacgcagtagcagtcacccagtgtttcaccgtggtgaattttctgtctgcgactccgtctctgtgtgggtaggggataagacgactgctaccgacatcaaggggaaggaagtgatggttttaggggaggtgaatattaacaattcggtcttcaagcaatacttcttcgaaacaaaatgccgcgatccaacgccagtggatagtggttgtcgcggtattgactcgaagcattggaactcatactgcaccacaacccatacctttgtgaaggcattgacaatggatggcaaacaggccgcctggcgttttattcgcattgacaccgcctgtgtctgtgtccttagtcgcaaagctggtcgccgcgcgggtggttgcgggtaactgcag

SEQ ID NO: 29 - protein, 229 aa, synthetic construct
MAEPHPESNVPAGHAIPQAHWTKLQHSLDTALRRARSTPAGAIAARVAGQTRNITVDPKLFKKRRLRSPRVLFSTHPPPVAADTQGLDLEAGGAASFNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCG

SEQ ID NO: 30 - protein, 121 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAG

SEQ ID NO: 31 - protein, 124 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCG

SEQ ID NO: 32 - DNA, 711 bp, synthetic construct
  source 1..711: mol_type = other DNA
atggccgaacctcacccagagtcaaacgttcccgcgggacatgcgattccacaggcgcactggacaaaacttcaacattcgttagacacagctcttcgccgtgcccgcagcacccctgcgggcgcaatcgcagcccgtgtagcgggtcaaacccgtaatattacagtggatccgaaattgtttaaaaagcgccgtctgcgtagtccccgcgtgttattctccacgcatcccccccctgttgccgcggatacccaaggcttagatctggaagctgggggagctgcatctttcaaccgcacgcaccgctcgaaacgcagtagcagtcacccagtgtttcaccgtggtgaattttctgtctgcgactccgtctctgtgtgggtaggggataagacgactgctaccgacatcaaggggaaggaagtgatggttttaggggaggtgaatattaacaattcggtcttcaagcaatacttcttcgaaacaaaatgccgcgatccaacgccagtggatagtggttgtcgcggtattgactcgaagcattggaactcatactgcaccacaacccatacctttgtgaaggcattgacaatggatggcaaacaggccgcctggcgttttattcgcattgacaccgcctgtgtctgtgtccttagtcgcaaagctggtcgccgcgcgggtggttgcgggaaacaccatcaccatcaccattaa

SEQ ID NO: 33 - protein, 131 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCGKHHHHHH

SEQ ID NO: 34 - protein, 6 aa, synthetic construct
HHHHHH

SEQ ID NO: 35 - DNA, 62 bp, synthetic construct
  source 1..62: mol_type = other DNA
atattctagaaataattttgtttaactttaagaaggagatataccatggccgaacctcaccc

SEQ ID NO: 36 - DNA, 37 bp, synthetic construct
  source 1..37: mol_type = other DNA
cattaagcttgtcgacctgcagttacccgcaaccacc

SEQ ID NO: 37 - protein, 15 aa, synthetic construct
GGGGSGGGGSGGGGS

SEQ ID NO: 38 - protein, 4 aa, synthetic construct
GGCG

SEQ ID NO: 39 - protein, 218 aa, synthetic construct
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 40 - protein, 27 aa, synthetic construct
DKSESTSAGRSRRRRPRRGSRSAPSSA

SEQ ID NO: 41 - protein, 14 aa, synthetic construct
QYIKANSKFIGITE

SEQ ID NO: 42 - protein, 13 aa, synthetic construct
AKFVAAWTLKAAA

SEQ ID NO: 43 - protein, 13 aa, synthetic construct
PKYVKQNTLKLAT

SEQ ID NO: 44 - protein, 20 aa, synthetic construct
PHHTALRQAILCWGELMTLA

SEQ ID NO: 45 - protein, 21 aa, synthetic construct
DIEKKIAKMEKASSVFNVVNS

SEQ ID NO: 46 - protein, 16 aa, synthetic construct
YSGPLKAEIAQRLEDV

SEQ ID NO: 47 - protein, 21 aa, synthetic construct
FNNFTVSFWLRVPKVSASHLE

SEQ ID NO: 48 - protein, 220 aa, synthetic construct
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 49 - protein, 19 aa, synthetic construct
GGGSEEEEEEEECGGGSGS

SEQ ID NO: 50 - protein, 14 aa, synthetic construct
GGGSEEEEGGGSGS

SEQ ID NO: 51 - protein, 18 aa, synthetic construct
GGGSEEEEEEEEGGGSGS

SEQ ID NO: 52 - DNA, 10 bp, synthetic construct
  source 1..10: mol_type = other DNA
gacgatcgtc

SEQ ID NO: 53 - DNA, 30 bp, synthetic construct
  source 1..30: mol_type = other DNA
gggggggggggacgatcgtcgggggggggg

SEQ ID NO: 54 - protein, 120 aa, synthetic construct
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 55 - protein, 121 aa, synthetic construct; identical to SEQ ID NO: 30

SEQ ID NO: 56 - protein, 120 aa, synthetic construct
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 57 - protein, 120 aa, synthetic construct
SSSHPVFHRGEFSVCDSISVWVGDKTTATDIKGKEVMVLGEVNINNSVFKQYFFETKCRDPNPVDSGCRGIDAKHWNSYCTTTHTFVKALTMDGKQAAWRFIRIDTACVCVLSRKTGQRA

SEQ ID NO: 58 - protein, 120 aa, synthetic construct
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAGRRA